SEQUENCE LISTING (565 sequences)

SEQ ID NO: 1 - DNA, 26 bp, Artificial Sequence
  Oligonucleotide Detection Strand
gcttgtctacacacacacacctggag

SEQ ID NO: 2 - DNA, 26 bp, Artificial Sequence
  Oligonucleotide Detection Strand
gcttgtctacacacatacacctggag

SEQ ID NO: 3 - DNA, 26 bp, Artificial Sequence
  Oligonucleotide Detection Strand
gcttgtctacacaccaacacctggag

SEQ ID NO: 4 - DNA, 26 bp, Artificial Sequence
  Oligonucleotide Detection Strand
gcttgtctacacacccacacctggag

SEQ ID NO: 5 - DNA, 26 bp, Artificial Sequence
  Oligonucleotide Detection Strand
gcttgtctacacacctacacctggag

SEQ ID NO: 6 - DNA, 26 bp, Artificial Sequence
  Oligonucleotide Detection Strand
gcttgtctacacacgaacacctggag

SEQ ID NO: 7 - DNA, 26 bp, Artificial Sequence
  Oligonucleotide Detection Strand
gcttgtctacacacgcacacctggag

SEQ ID NO: 8 - DNA, 26 bp, Artificial Sequence
  Oligonucleotide Detection Strand
gcttgtctacacactaacacctggag

SEQ ID NO: 9 - DNA, 26 bp, Artificial Sequence
  Oligonucleotide Detection Strand
gcttgtctacacactcacacctggag

SEQ ID NO: 10 - DNA, 26 bp, Artificial Sequence
  Oligonucleotide Detection Strand
gcttgtctacacacttacacctggag

SEQ ID NO: 11 - DNA, 26 bp, Artificial Sequence
  Oligonucleotide Detection Strand
gcttgtctacacatacacacctggag

SEQ ID NO: 12 - DNA, 26 bp, Artificial Sequence
  Oligonucleotide Detection Strand
gcttgtctacacatcaacacctggag

SEQ ID NO: 13 - DNA, 26 bp, Artificial Sequence
  Oligonucleotide Detection Strand
gcttgtctacacatctacacctggag

SEQ ID NO: 14 - DNA, 26 bp, Artificial Sequence
  Oligonucleotide Detection Strand
gcttgtctacaccaaaacacctggag

SEQ ID NO: 15 - DNA, 26 bp, Artificial Sequence
  Oligonucleotide Detection Strand
gcttgtctacaccaacacacctggag

SEQ ID NO: 16 - DNA, 26 bp, Artificial Sequence
  Oligonucleotide Detection Strand
gcttgtctacaccaatacacctggag

SEQ ID NO: 17 - DNA, 26 bp, Artificial Sequence
  Oligonucleotide Detection Strand
gcttgtctacaccaccacacctggag

SEQ ID NO: 18 - DNA, 26 bp, Artificial Sequence
  Oligonucleotide Detection Strand
gcttgtctacaccactacacctggag

SEQ ID NO: 19 - DNA, 26 bp, Artificial Sequence
  Oligonucleotide Detection Strand
gcttgtctacaccataacacctggag

SEQ ID NO: 20 - DNA, 26 bp, Artificial Sequence
  Oligonucleotide Detection Strand
gcttgtctacaccatcacacctggag

SEQ ID NO: 21 - DNA, 26 bp, Artificial Sequence
  Oligonucleotide Detection Strand
gcttgtctacaccattacacctggag

SEQ ID NO: 22 - DNA, 26 bp, Artificial Sequence
  Oligonucleotide Detection Strand
gcttgtctacacccaaacacctggag

SEQ ID NO: 23 - DNA, 26 bp, Artificial Sequence
  Oligonucleotide Detection Strand
gcttgtctacacccacacacctggag

SEQ ID NO: 24 - DNA, 26 bp, Artificial Sequence
  Oligonucleotide Detection Strand
gcttgtctacacccatacacctggag

SEQ ID NO: 25 - DNA, 26 bp, Artificial Sequence
  Oligonucleotide Detection Strand
gcttgtctacaccccaacacctggag

SEQ ID NO: 26 - DNA, 26 bp, Artificial Sequence
  Oligonucleotide Detection Strand
gcttgtctacacccctacacctggag

SEQ ID NO: 27 - DNA, 26 bp, Artificial Sequence
  Oligonucleotide Detection Strand
gcttgtctacacccgaacacctggag

SEQ ID NO: 28 - DNA, 26 bp, Artificial Sequence
  Oligonucleotide Detection Strand
gcttgtctacaccctaacacctggag

SEQ ID NO: 29 - DNA, 26 bp, Artificial Sequence
  Oligonucleotide Detection Strand
gcttgtctacaccctcacacctggag

SEQ ID NO: 30 - DNA, 26 bp, Artificial Sequence
  Oligonucleotide Detection Strand
gcttgtctacacccttacacctggag

SEQ ID NO: 31 - DNA, 26 bp, Artificial Sequence
  Oligonucleotide Detection Strand
gcttgtctacaccgaaacacctggag

SEQ ID NO: 32 - DNA, 26 bp, Artificial Sequence
  Oligonucleotide Detection Strand
gcttgtctacaccgcaacacctggag

SEQ ID NO: 33 - DNA, 26 bp, Artificial Sequence
  Oligonucleotide Detection Strand
gcttgtctacaccgctacacctggag

SEQ ID NO: 34 - DNA, 26 bp, Artificial Sequence
  Oligonucleotide Detection Strand
gcttgtctacacctaaacacctggag

SEQ ID NO: 35 - DNA, 26 bp, Artificial Sequence
  Oligonucleotide Detection Strand
gcttgtctacacctacacacctggag

SEQ ID NO: 36 - DNA, 26 bp, Artificial Sequence
  Oligonucleotide Detection Strand
gcttgtctacacctcaacacctggag

SEQ ID NO: 37 - DNA, 26 bp, Artificial Sequence
  Oligonucleotide Detection Strand
gcttgtctacacctctacacctggag

SEQ ID NO: 38 - DNA, 26 bp, Artificial Sequence
  Oligonucleotide Detection Strand
gcttgtctacaccttcacacctggag

SEQ ID NO: 39 - DNA, 26 bp, Artificial Sequence
  Oligonucleotide Detection Strand
gcttgtctacacgaaaacacctggag

SEQ ID NO: 40 - DNA, 26 bp, Artificial Sequence
  Oligonucleotide Detection Strand
gcttgtctacacgaacacacctggag

SEQ ID NO: 41 - DNA, 26 bp, Artificial Sequence
  Oligonucleotide Detection Strand
gcttgtctacacgaatacacctggag

SEQ ID NO: 42 - DNA, 26 bp, Artificial Sequence
  Oligonucleotide Detection Strand
gcttgtctacacgaccacacctggag

SEQ ID NO: 43 - DNA, 26 bp, Artificial Sequence
  Oligonucleotide Detection Strand
gcttgtctacacgactacacctggag

SEQ ID NO: 44 - DNA, 26 bp, Artificial Sequence
  Oligonucleotide Detection Strand
gcttgtctacacgcaaacacctggag

SEQ ID NO: 45 - DNA, 26 bp, Artificial Sequence
  Oligonucleotide Detection Strand
gcttgtctacacgcacacacctggag

SEQ ID NO: 46 - DNA, 26 bp, Artificial Sequence
  Oligonucleotide Detection Strand
gcttgtctacacgcatacacctggag

SEQ ID NO: 47 - DNA, 26 bp, Artificial Sequence
  Oligonucleotide Detection Strand
gcttgtctacacgccaacacctggag

SEQ ID NO: 48 - DNA, 26 bp, Artificial Sequence
  Oligonucleotide Detection Strand
gcttgtctacacgcctacacctggag

SEQ ID NO: 49 - DNA, 26 bp, Artificial Sequence
  Oligonucleotide Detection Strand
gcttgtctacacgctaacacctggag

SEQ ID NO: 50 - DNA, 26 bp, Artificial Sequence
  Oligonucleotide Detection Strand
gcttgtctacacgctcacacctggag

SEQ ID NO: 51 - DNA, 26 bp, Artificial Sequence
  Oligonucleotide Detection Strand
gcttgtctacacgcttacacctggag

SEQ ID NO: 52 - DNA, 26 bp, Artificial Sequence
  Oligonucleotide Detection Strand
gcttgtctacacggaaacacctggag

SEQ ID NO: 53 - DNA, 26 bp, Artificial Sequence
  Oligonucleotide Detection Strand
gcttgtctacacggctacacctggag

SEQ ID NO: 54 - DNA, 26 bp, Artificial Sequence
  Oligonucleotide Detection Strand
gcttgtctacactaacacacctggag

SEQ ID NO: 55 - DNA, 26 bp, Artificial Sequence
  Oligonucleotide Detection Strand
gcttgtctacactaccacacctggag

SEQ ID NO: 56 - DNA, 26 bp, Artificial Sequence
  Oligonucleotide Detection Strand
gcttgtctacactactacacctggag

SEQ ID NO: 57 - DNA, 26 bp, Artificial Sequence
  Oligonucleotide Detection Strand
gcttgtctacactatcacacctggag

SEQ ID NO: 58 - DNA, 26 bp, Artificial Sequence
  Oligonucleotide Detection Strand
gcttgtctacactcaaacacctggag

SEQ ID NO: 59 - DNA, 26 bp, Artificial Sequence
  Oligonucleotide Detection Strand
gcttgtctacactcacacacctggag

SEQ ID NO: 60 - DNA, 26 bp, Artificial Sequence
  Oligonucleotide Detection Strand
gcttgtctacactcatacacctggag

SEQ ID NO: 61 - DNA, 26 bp, Artificial Sequence
  Oligonucleotide Detection Strand
gcttgtctacactcgcacacctggag

SEQ ID NO: 62 - DNA, 26 bp, Artificial Sequence
  Oligonucleotide Detection Strand
gcttgtctacactctaacacctggag

SEQ ID NO: 63 - DNA, 26 bp, Artificial Sequence
  Oligonucleotide Detection Strand
gcttgtctacactctcacacctggag

SEQ ID NO: 64 - DNA, 26 bp, Artificial Sequence
  Oligonucleotide Detection Strand
gcttgtctacactcttacacctggag

SEQ ID NO: 65 - DNA, 26 bp, Artificial Sequence
  Oligonucleotide Detection Strand
gcttgtctacactgccacacctggag

SEQ ID NO: 66 - DNA, 26 bp, Artificial Sequence
  Oligonucleotide Detection Strand
gcttgtctacactgctacacctggag

SEQ ID NO: 67 - DNA, 26 bp, Artificial Sequence
  Oligonucleotide Detection Strand
gcttgtctacactggaacacctggag

SEQ ID NO: 68 - DNA, 26 bp, Artificial Sequence
  Oligonucleotide Detection Strand
gcttgtctacactgtcacacctggag

SEQ ID NO: 69 - DNA, 26 bp, Artificial Sequence
  Oligonucleotide Detection Strand
gcttgtctacacttacacacctggag

SEQ ID NO: 70 - DNA, 26 bp, Artificial Sequence
  Oligonucleotide Detection Strand
gcttgtctacacttcaacacctggag

SEQ ID NO: 71 - DNA, 26 bp, Artificial Sequence
  Oligonucleotide Detection Strand
gcttgtctacacttctacacctggag

SEQ ID NO: 72 - DNA, 26 bp, Artificial Sequence
  Oligonucleotide Detection Strand
gcttgtctacataaccacacctggag

SEQ ID NO: 73 - DNA, 26 bp, Artificial Sequence
  Oligonucleotide Detection Strand
gcttgtctacatacacacacctggag

SEQ ID NO: 74 - DNA, 26 bp, Artificial Sequence
  Oligonucleotide Detection Strand
gcttgtctacataccaacacctggag

SEQ ID NO: 75 - DNA, 26 bp, Artificial Sequence
  Oligonucleotide Detection Strand
gcttgtctacatacccacacctggag

SEQ ID NO: 76 - DNA, 26 bp, Artificial Sequence
  Oligonucleotide Detection Strand
gcttgtctacatacctacacctggag

SEQ ID NO: 77 - DNA, 26 bp, Artificial Sequence
  Oligonucleotide Detection Strand
gcttgtctacatacgaacacctggag

SEQ ID NO: 78 - DNA, 26 bp, Artificial Sequence
  Oligonucleotide Detection Strand
gcttgtctacatacgcacacctggag

SEQ ID NO: 79 - DNA, 26 bp, Artificial Sequence
  Oligonucleotide Detection Strand
gcttgtctacatactcacacctggag

SEQ ID NO: 80 - DNA, 26 bp, Artificial Sequence
  Oligonucleotide Detection Strand
gcttgtctacatcaacacacctggag

SEQ ID NO: 81 - DNA, 26 bp, Artificial Sequence
  Oligonucleotide Detection Strand
gcttgtctacatcaccacacctggag

SEQ ID NO: 82 - DNA, 26 bp, Artificial Sequence
  Oligonucleotide Detection Strand
gcttgtctacatcactacacctggag

SEQ ID NO: 83 - DNA, 26 bp, Artificial Sequence
  Oligonucleotide Detection Strand
gcttgtctacatcatcacacctggag

SEQ ID NO: 84 - DNA, 26 bp, Artificial Sequence
  Oligonucleotide Detection Strand
gcttgtctacatcccaacacctggag

SEQ ID NO: 85 - DNA, 26 bp, Artificial Sequence
  Oligonucleotide Detection Strand
gcttgtctacatccccacacctggag

SEQ ID NO: 86 - DNA, 26 bp, Artificial Sequence
  Oligonucleotide Detection Strand
gcttgtctacatccctacacctggag

SEQ ID NO: 87 - DNA, 26 bp, Artificial Sequence
  Oligonucleotide Detection Strand
gcttgtctacatcctaacacctggag

SEQ ID NO: 88 - DNA, 26 bp, Artificial Sequence
  Oligonucleotide Detection Strand
gcttgtctacatcctcacacctggag

SEQ ID NO: 89 - DNA, 26 bp, Artificial Sequence
  Oligonucleotide Detection Strand
gcttgtctacatccttacacctggag

SEQ ID NO: 90 - DNA, 26 bp, Artificial Sequence
  Oligonucleotide Detection Strand
gcttgtctacatcgcaacacctggag

SEQ ID NO: 91 - DNA, 26 bp, Artificial Sequence
  Oligonucleotide Detection Strand
gcttgtctacatcgccacacctggag

SEQ ID NO: 92 - DNA, 26 bp, Artificial Sequence
  Oligonucleotide Detection Strand
gcttgtctacatcgctacacctggag

SEQ ID NO: 93 - DNA, 26 bp, Artificial Sequence
  Oligonucleotide Detection Strand
gcttgtctacatctacacacctggag

SEQ ID NO: 94 - DNA, 26 bp, Artificial Sequence
  Oligonucleotide Detection Strand
gcttgtctacatctcaacacctggag

SEQ ID NO: 95 - DNA, 26 bp, Artificial Sequence
  Oligonucleotide Detection Strand
gcttgtctacatctctacacctggag

SEQ ID NO: 96 - DNA, 26 bp, Artificial Sequence
  Oligonucleotide Detection Strand
gcttgtctacatcttcacacctggag

SEQ ID NO: 97 - DNA, 26 bp, Artificial Sequence
  Oligonucleotide Detection Strand
gcttgtctacattaccacacctggag

SEQ ID NO: 98 - DNA, 26 bp, Artificial Sequence
  Oligonucleotide Detection Strand
gcttgtctaccaaaacacacctggag

SEQ ID NO: 99 - DNA, 26 bp, Artificial Sequence
  Oligonucleotide Detection Strand
gcttgtctaccaaaccacacctggag

SEQ ID NO: 100 - DNA, 26 bp, Artificial Sequence
  Oligonucleotide Detection Strand
gcttgtctaccaaactacacctggag

SEQ ID NO: 101 - DNA, 26 bp, Artificial Sequence
  Oligonucleotide Detection Strand
gcttgtctaccaaatcacacctggag

SEQ ID NO: 102 - DNA, 26 bp, Artificial Sequence
  Oligonucleotide Detection Strand
gcttgtctaccaacacacacctggag

SEQ ID NO: 103 - DNA, 26 bp, Artificial Sequence
  Oligonucleotide Detection Strand
gcttgtctaccaacatacacctggag

SEQ ID NO: 104 - DNA, 26 bp, Artificial Sequence
  Oligonucleotide Detection Strand
gcttgtctaccaaccaacacctggag

SEQ ID NO: 105 - DNA, 26 bp, Artificial Sequence
  Oligonucleotide Detection Strand
gcttgtctaccaacccacacctggag

SEQ ID NO: 106 - DNA, 26 bp, Artificial Sequence
  Oligonucleotide Detection Strand
gcttgtctaccaacctacacctggag

SEQ ID NO: 107 - DNA, 26 bp, Artificial Sequence
  Oligonucleotide Detection Strand
gcttgtctaccaacgaacacctggag

SEQ ID NO: 108 - DNA, 26 bp, Artificial Sequence
  Oligonucleotide Detection Strand
gcttgtctaccaacgcacacctggag

SEQ ID NO: 109 - DNA, 26 bp, Artificial Sequence
  Oligonucleotide Detection Strand
gcttgtctaccaactaacacctggag

SEQ ID NO: 110 - DNA, 26 bp, Artificial Sequence
  Oligonucleotide Detection Strand
gcttgtctaccaactcacacctggag

SEQ ID NO: 111 - DNA, 26 bp, Artificial Sequence
  Oligonucleotide Detection Strand
gcttgtctaccaacttacacctggag

SEQ ID NO: 112 - DNA, 26 bp, Artificial Sequence
  Oligonucleotide Detection Strand
gcttgtctaccaatacacacctggag

SEQ ID NO: 113 - DNA, 26 bp, Artificial Sequence
  Oligonucleotide Detection Strand
gcttgtctaccaatcaacacctggag

SEQ ID NO: 114 - DNA, 26 bp, Artificial Sequence
  Oligonucleotide Detection Strand
gcttgtctaccaatctacacctggag

SEQ ID NO: 115 - DNA, 26 bp, Artificial Sequence
  Oligonucleotide Detection Strand
gcttgtctaccacaccacacctggag

SEQ ID NO: 116 - DNA, 26 bp, Artificial Sequence
  Oligonucleotide Detection Strand
gcttgtctaccacactacacctggag

SEQ ID NO: 117 - DNA, 26 bp, Artificial Sequence
  Oligonucleotide Detection Strand
gcttgtctaccacataacacctggag

SEQ ID NO: 118 - DNA, 26 bp, Artificial Sequence
  Oligonucleotide Detection Strand
gcttgtctaccacatcacacctggag

SEQ ID NO: 119 - DNA, 26 bp, Artificial Sequence
  Oligonucleotide Detection Strand
gcttgtctaccacattacacctggag

SEQ ID NO: 120 - DNA, 26 bp, Artificial Sequence
  Oligonucleotide Detection Strand
gcttgtctaccaccaaacacctggag

SEQ ID NO: 121 - DNA, 26 bp, Artificial Sequence
  Oligonucleotide Detection Strand
gcttgtctaccaccacacacctggag

SEQ ID NO: 122 - DNA, 26 bp, Artificial Sequence
  Oligonucleotide Detection Strand
gcttgtctaccaccatacacctggag

SEQ ID NO: 123 - DNA, 26 bp, Artificial Sequence
  Oligonucleotide Detection Strand
gcttgtctaccacccaacacctggag

SEQ ID NO: 124 - DNA, 26 bp, Artificial Sequence
  Oligonucleotide Detection Strand
gcttgtctaccaccctacacctggag

SEQ ID NO: 125 - DNA, 26 bp, Artificial Sequence
  Oligonucleotide Detection Strand
gcttgtctaccaccgaacacctggag

SEQ ID NO: 126 - DNA, 26 bp, Artificial Sequence
  Oligonucleotide Detection Strand
gcttgtctaccacctaacacctggag

SEQ ID NO: 127 - DNA, 26 bp, Artificial Sequence
  Oligonucleotide Detection Strand
gcttgtctaccacctcacacctggag

SEQ ID NO: 128 - DNA, 26 bp, Artificial Sequence
  Oligonucleotide Detection Strand
gcttgtctaccaccttacacctggag

SEQ ID NO: 129 - DNA, 26 bp, Artificial Sequence
  Oligonucleotide Detection Strand
gcttgtctaccacgaaacacctggag

SEQ ID NO: 130 - DNA, 26 bp, Artificial Sequence
  Oligonucleotide Detection Strand
gcttgtctaccacgcaacacctggag

SEQ ID NO: 131 - DNA, 26 bp, Artificial Sequence
  Oligonucleotide Detection Strand
gcttgtctaccacgctacacctggag

SEQ ID NO: 132 - DNA, 26 bp, Artificial Sequence
  Oligonucleotide Detection Strand
gcttgtctaccacggaacacctggag

SEQ ID NO: 133 - DNA, 26 bp, Artificial Sequence
  Oligonucleotide Detection Strand
gcttgtctaccactaaacacctggag

SEQ ID NO: 134 - DNA, 26 bp, Artificial Sequence
  Oligonucleotide Detection Strand
gcttgtctaccactacacacctggag

SEQ ID NO: 135 - DNA, 26 bp, Artificial Sequence
  Oligonucleotide Detection Strand
gcttgtctaccactcaacacctggag

SEQ ID NO: 136 - DNA, 26 bp, Artificial Sequence
  Oligonucleotide Detection Strand
gcttgtctaccactctacacctggag

SEQ ID NO: 137 - DNA, 26 bp, Artificial Sequence
  Oligonucleotide Detection Strand
gcttgtctaccacttcacacctggag

SEQ ID NO: 138 - DNA, 26 bp, Artificial Sequence
  Oligonucleotide Detection Strand
gcttgtctaccataacacacctggag

SEQ ID NO: 139 - DNA, 26 bp, Artificial Sequence
  Oligonucleotide Detection Strand
gcttgtctaccataccacacctggag

SEQ ID NO: 140 - DNA, 26 bp, Artificial Sequence
  Oligonucleotide Detection Strand
gcttgtctaccatactacacctggag

SEQ ID NO: 141 - DNA, 26 bp, Artificial Sequence
  Oligonucleotide Detection Strand
gcttgtctaccatcaaacacctggag

SEQ ID NO: 142 - DNA, 26 bp, Artificial Sequence
  Oligonucleotide Detection Strand
gcttgtctaccatcacacacctggag

SEQ ID NO: 143 - DNA, 26 bp, Artificial Sequence
  Oligonucleotide Detection Strand
gcttgtctaccatcatacacctggag

SEQ ID NO: 144 - DNA, 26 bp, Artificial Sequence
  Oligonucleotide Detection Strand
gcttgtctaccatcccacacctggag

SEQ ID NO: 145 - DNA, 26 bp, Artificial Sequence
  Oligonucleotide Detection Strand
gcttgtctaccatcctacacctggag

SEQ ID NO: 146 - DNA, 26 bp, Artificial Sequence
  Oligonucleotide Detection Strand
gcttgtctaccatcgcacacctggag

SEQ ID NO: 147 - DNA, 26 bp, Artificial Sequence
  Oligonucleotide Detection Strand
gcttgtctaccatctaacacctggag

SEQ ID NO: 148 - DNA, 26 bp, Artificial Sequence
  Oligonucleotide Detection Strand
gcttgtctaccatctcacacctggag

SEQ ID NO: 149 - DNA, 26 bp, Artificial Sequence
  Oligonucleotide Detection Strand
gcttgtctaccatcttacacctggag

SEQ ID NO: 150 - DNA, 26 bp, Artificial Sequence
  Oligonucleotide Detection Strand
gcttgtctaccattacacacctggag

SEQ ID NO: 151 - DNA, 26 bp, Artificial Sequence
  Oligonucleotide Detection Strand
gcttgtctacccaaaaacacctggag

SEQ ID NO: 152 - DNA, 26 bp, Artificial Sequence
  Oligonucleotide Detection Strand
gcttgtctacccaaacacacctggag

SEQ ID NO: 153 - DNA, 26 bp, Artificial Sequence
  Oligonucleotide Detection Strand
gcttgtctacccaaatacacctggag

SEQ ID NO: 154 - DNA, 26 bp, Artificial Sequence
  Oligonucleotide Detection Strand
gcttgtctacccaaccacacctggag

SEQ ID NO: 155 - DNA, 26 bp, Artificial Sequence
  Oligonucleotide Detection Strand
gcttgtctacccaactacacctggag

SEQ ID NO: 156 - DNA, 26 bp, Artificial Sequence
  Oligonucleotide Detection Strand
gcttgtctacccaataacacctggag

SEQ ID NO: 157 - DNA, 26 bp, Artificial Sequence
  Oligonucleotide Detection Strand
gcttgtctacccaatcacacctggag

SEQ ID NO: 158 - DNA, 26 bp, Artificial Sequence
  Oligonucleotide Detection Strand
gcttgtctacccacacacacctggag

SEQ ID NO: 159 - DNA, 26 bp, Artificial Sequence
  Oligonucleotide Detection Strand
gcttgtctacccacatacacctggag

SEQ ID NO: 160 - DNA, 26 bp, Artificial Sequence
  Oligonucleotide Detection Strand
gcttgtctacccaccaacacctggag

SEQ ID NO: 161 - DNA, 26 bp, Artificial Sequence
  Oligonucleotide Detection Strand
gcttgtctacccacctacacctggag

SEQ ID NO: 162 - DNA, 26 bp, Artificial Sequence
  Oligonucleotide Detection Strand
gcttgtctacccacgaacacctggag

SEQ ID NO: 163 - DNA, 26 bp, Artificial Sequence
  Oligonucleotide Detection Strand
gcttgtctacccactaacacctggag

SEQ ID NO: 164 - DNA, 26 bp, Artificial Sequence
  Oligonucleotide Detection Strand
gcttgtctacccactcacacctggag

SEQ ID NO: 165 - DNA, 26 bp, Artificial Sequence
  Oligonucleotide Detection Strand
gcttgtctacccacttacacctggag

SEQ ID NO: 166 - DNA, 26 bp, Artificial Sequence
  Oligonucleotide Detection Strand
gcttgtctacccataaacacctggag

SEQ ID NO: 167 - DNA, 26 bp, Artificial Sequence
  Oligonucleotide Detection Strand
gcttgtctacccatacacacctggag

SEQ ID NO: 168 - DNA, 26 bp, Artificial Sequence
  Oligonucleotide Detection Strand
gcttgtctacccatcaacacctggag

SEQ ID NO: 169 - DNA, 26 bp, Artificial Sequence
  Oligonucleotide Detection Strand
gcttgtctacccatctacacctggag

SEQ ID NO: 170 - DNA, 26 bp, Artificial Sequence
  Oligonucleotide Detection Strand
gcttgtctaccccaaaacacctggag

SEQ ID NO: 171 - DNA, 26 bp, Artificial Sequence
  Oligonucleotide Detection Strand
gcttgtctaccccaacacacctggag

SEQ ID NO: 172 - DNA, 26 bp, Artificial Sequence
  Oligonucleotide Detection Strand
gcttgtctaccccaatacacctggag

SEQ ID NO: 173 - DNA, 26 bp, Artificial Sequence
  Oligonucleotide Detection Strand
gcttgtctaccccactacacctggag

SEQ ID NO: 174 - DNA, 26 bp, Artificial Sequence
  Oligonucleotide Detection Strand
gcttgtctaccccataacacctggag

SEQ ID NO: 175 - DNA, 26 bp, Artificial Sequence
  Oligonucleotide Detection Strand
gcttgtctaccccatcacacctggag

SEQ ID NO: 176 - DNA, 26 bp, Artificial Sequence
  Oligonucleotide Detection Strand
gcttgtctaccccattacacctggag

SEQ ID NO: 177 - DNA, 26 bp, Artificial Sequence
  Oligonucleotide Detection Strand
gcttgtctacccccaaacacctggag

SEQ ID NO: 178 - DNA, 26 bp, Artificial Sequence
  Oligonucleotide Detection Strand
gcttgtctacccccatacacctggag

SEQ ID NO: 179 - DNA, 26 bp, Artificial Sequence
  Oligonucleotide Detection Strand
gcttgtctaccccctaacacctggag

SEQ ID NO: 180 - DNA, 26 bp, Artificial Sequence
  Oligonucleotide Detection Strand
gcttgtctacccccttacacctggag

SEQ ID NO: 181 - DNA, 26 bp, Artificial Sequence
  Oligonucleotide Detection Strand
gcttgtctaccccgaaacacctggag

SEQ ID NO: 182 - DNA, 26 bp, Artificial Sequence
  Oligonucleotide Detection Strand
gcttgtctacccctaaacacctggag

SEQ ID NO: 183 - DNA, 26 bp, Artificial Sequence
  Oligonucleotide Detection Strand
gcttgtctacccctacacacctggag

SEQ ID NO: 184 - DNA, 26 bp, Artificial Sequence
  Oligonucleotide Detection Strand
gcttgtctacccctcaacacctggag

SEQ ID NO: 185 - DNA, 26 bp, Artificial Sequence
  Oligonucleotide Detection Strand
gcttgtctacccctctacacctggag

SEQ ID NO: 186 - DNA, 26 bp, Artificial Sequence
  Oligonucleotide Detection Strand
gcttgtctaccccttcacacctggag

SEQ ID NO: 187 - DNA, 26 bp, Artificial Sequence
  Oligonucleotide Detection Strand
gcttgtctacccgaaaacacctggag

SEQ ID NO: 188 - DNA, 26 bp, Artificial Sequence
  Oligonucleotide Detection Strand
gcttgtctacccgaacacacctggag

SEQ ID NO: 189 - DNA, 26 bp, Artificial Sequence
  Oligonucleotide Detection Strand
gcttgtctacccgaatacacctggag

SEQ ID NO: 190 - DNA, 26 bp, Artificial Sequence
  Oligonucleotide Detection Strand
gcttgtctacccgactacacctggag

SEQ ID NO: 191 - DNA, 26 bp, Artificial Sequence
  Oligonucleotide Detection Strand
gcttgtctacccgcaaacacctggag

SEQ ID NO: 192 - DNA, 26 bp, Artificial Sequence
  Oligonucleotide Detection Strand
gcttgtctacccgcatacacctggag

SEQ ID NO: 193 - DNA, 26 bp, Artificial Sequence
  Oligonucleotide Detection Strand
gcttgtctacccgctaacacctggag

SEQ ID NO: 194 - DNA, 26 bp, Artificial Sequence
  Oligonucleotide Detection Strand
gcttgtctacccgcttacacctggag

SEQ ID NO: 195 - DNA, 26 bp, Artificial Sequence
  Oligonucleotide Detection Strand
gcttgtctaccctaaaacacctggag

SEQ ID NO: 196 - DNA, 26 bp, Artificial Sequence
  Oligonucleotide Detection Strand
gcttgtctaccctaacacacctggag

SEQ ID NO: 197 - DNA, 26 bp, Artificial Sequence
  Oligonucleotide Detection Strand
gcttgtctaccctaccacacctggag

SEQ ID NO: 198 - DNA, 26 bp, Artificial Sequence
  Oligonucleotide Detection Strand
gcttgtctaccctactacacctggag

SEQ ID NO: 199 - DNA, 26 bp, Artificial Sequence
  Oligonucleotide Detection Strand
gcttgtctaccctatcacacctggag

SEQ ID NO: 200 - DNA, 26 bp, Artificial Sequence
  Oligonucleotide Detection Strand
gcttgtctaccctcaaacacctggag

SEQ ID NO: 201 - DNA, 26 bp, Artificial Sequence
  Oligonucleotide Detection Strand
gcttgtctaccctcacacacctggag

SEQ ID NO: 202 - DNA, 26 bp, Artificial Sequence
  Oligonucleotide Detection Strand
gcttgtctaccctcatacacctggag

SEQ ID NO: 203 - DNA, 26 bp, Artificial Sequence
  Oligonucleotide Detection Strand
gcttgtctaccctctaacacctggag

SEQ ID NO: 204 - DNA, 26 bp, Artificial Sequence
  Oligonucleotide Detection Strand
gcttgtctaccctctcacacctggag

SEQ ID NO: 205 - DNA, 26 bp, Artificial Sequence
  Oligonucleotide Detection Strand
gcttgtctaccctcttacacctggag

SEQ ID NO: 206 - DNA, 26 bp, Artificial Sequence
  Oligonucleotide Detection Strand
gcttgtctaccctgctacacctggag

SEQ ID NO: 207 - DNA, 26 bp, Artificial Sequence
  Oligonucleotide Detection Strand
gcttgtctaccctggaacacctggag

SEQ ID NO: 208 - DNA, 26 bp, Artificial Sequence
  Oligonucleotide Detection Strand
gcttgtctaccctgtcacacctggag

SEQ ID NO: 209 - DNA, 26 bp, Artificial Sequence
  Oligonucleotide Detection Strand
gcttgtctacccttacacacctggag

SEQ ID NO: 210 - DNA, 26 bp, Artificial Sequence
  Oligonucleotide Detection Strand
gcttgtctacccttcaacacctggag

SEQ ID NO: 211 - DNA, 26 bp, Artificial Sequence
  Oligonucleotide Detection Strand
gcttgtctacccttctacacctggag

SEQ ID NO: 212 - DNA, 26 bp, Artificial Sequence
  Oligonucleotide Detection Strand
gcttgtctaccgaaaaacacctggag

SEQ ID NO: 213 - DNA, 24 bp, Artificial Sequence
  Oligonucleotide Detection Strand
gcgtctaccgaaacacacctggag

SEQ ID NO: 214 - DNA, 26 bp, Artificial Sequence
  Oligonucleotide Detection Strand
gcttgtctaccgaaatacacctggag

SEQ ID NO: 215 - DNA, 26 bp, Artificial Sequence
  Oligonucleotide Detection Strand
gcttgtctaccgaaccacacctggag

SEQ ID NO: 216 - DNA, 26 bp, Artificial Sequence
  Oligonucleotide Detection Strand
gcttgtctaccgaactacacctggag

SEQ ID NO: 217 - DNA, 26 bp, Artificial Sequence
  Oligonucleotide Detection Strand
gcttgtctaccgaataacacctggag

SEQ ID NO: 218 - DNA, 26 bp, Artificial Sequence
  Oligonucleotide Detection Strand
gcttgtctaccgaatcacacctggag

SEQ ID NO: 219 - DNA, 26 bp, Artificial Sequence
  Oligonucleotide Detection Strand
gcttgtctaccgaccaacacctggag

SEQ ID NO: 220 - DNA, 26 bp, Artificial Sequence
  Oligonucleotide Detection Strand
gcttgtctaccgacctacacctggag

SEQ ID NO: 221 - DNA, 26 bp, Artificial Sequence
  Oligonucleotide Detection Strand
gcttgtctaccgacgaacacctggag

SEQ ID NO: 222 - DNA, 26 bp, Artificial Sequence
  Oligonucleotide Detection Strand
gcttgtctaccgactaacacctggag

SEQ ID NO: 223 - DNA, 26 bp, Artificial Sequence
  Oligonucleotide Detection Strand
gcttgtctaccgactcacacctggag

SEQ ID NO: 224 - DNA, 26 bp, Artificial Sequence
  Oligonucleotide Detection Strand
gcttgtctaccgacttacacctggag

SEQ ID NO: 225 - DNA, 26 bp, Artificial Sequence
  Oligonucleotide Detection Strand
gcttgtctaccgcaaaacacctggag

SEQ ID NO: 226 - DNA, 26 bp, Artificial Sequence
  Oligonucleotide Detection Strand
gcttgtctaccgcaacacacctggag

SEQ ID NO: 227 - DNA, 26 bp, Artificial Sequence
  Oligonucleotide Detection Strand
gcttgtctaccgcaatacacctggag

SEQ ID NO: 228 - DNA, 26 bp, Artificial Sequence
  Oligonucleotide Detection Strand
gcttgtctaccgcactacacctggag

SEQ ID NO: 229 - DNA, 26 bp, Artificial Sequence
  Oligonucleotide Detection Strand
gcttgtctaccgcataacacctggag

SEQ ID NO: 230 - DNA, 26 bp, Artificial Sequence
  Oligonucleotide Detection Strand
gcttgtctaccgcatcacacctggag

SEQ ID NO: 231 - DNA, 26 bp, Artificial Sequence
  Oligonucleotide Detection Strand
gcttgtctaccgcattacacctggag

SEQ ID NO: 232 - DNA, 26 bp, Artificial Sequence
  Oligonucleotide Detection Strand
gcttgtctaccgccaaacacctggag

SEQ ID NO: 233 - DNA, 26 bp, Artificial Sequence
  Oligonucleotide Detection Strand
gcttgtctaccgccatacacctggag

SEQ ID NO: 234 - DNA, 26 bp, Artificial Sequence
  Oligonucleotide Detection Strand
gcttgtctaccgcctaacacctggag

SEQ ID NO: 235 - DNA, 26 bp, Artificial Sequence
  Oligonucleotide Detection Strand
gcttgtctaccgccttacacctggag

SEQ ID NO: 236 - DNA, 26 bp, Artificial Sequence
  Oligonucleotide Detection Strand
gcttgtctaccgctaaacacctggag

SEQ ID NO: 237 - DNA, 26 bp, Artificial Sequence
  Oligonucleotide Detection Strand
gcttgtctaccgctacacacctggag

SEQ ID NO: 238 - DNA, 26 bp, Artificial Sequence
  Oligonucleotide Detection Strand
gcttgtctaccgctcaacacctggag

SEQ ID NO: 239 - DNA, 26 bp, Artificial Sequence
  Oligonucleotide Detection Strand
gcttgtctaccgctctacacctggag

SEQ ID NO: 240 - DNA, 26 bp, Artificial Sequence
  Oligonucleotide Detection Strand
gcttgtctaccgcttcacacctggag

SEQ ID NO: 241 - DNA, 26 bp, Artificial Sequence
  Oligonucleotide Detection Strand
gcttgtctacctaaacacacctggag

SEQ ID NO: 242 - DNA, 26 bp, Artificial Sequence
  Oligonucleotide Detection Strand
gcttgtctacctaaccacacctggag

SEQ ID NO: 243 - DNA, 26 bp, Artificial Sequence
  Oligonucleotide Detection Strand
gcttgtctacctaactacacctggag

SEQ ID NO: 244 - DNA, 26 bp, Artificial Sequence
  Oligonucleotide Detection Strand
gcttgtctacctacacacacctggag

SEQ ID NO: 245 - DNA, 26 bp, Artificial Sequence
  Oligonucleotide Detection Strand
gcttgtctacctacatacacctggag

SEQ ID NO: 246 - DNA, 26 bp, Artificial Sequence
  Oligonucleotide Detection Strand
gcttgtctacctaccaacacctggag

SEQ ID NO: 247 - DNA, 26 bp, Artificial Sequence
  Oligonucleotide Detection Strand
gcttgtctacctacccacacctggag

SEQ ID NO: 248 - DNA, 26 bp, Artificial Sequence
  Oligonucleotide Detection Strand
gcttgtctacctacctacacctggag

SEQ ID NO: 249 - DNA, 26 bp, Artificial Sequence
  Oligonucleotide Detection Strand
gcttgtctacctacgaacacctggag

SEQ ID NO: 250 - DNA, 26 bp, Artificial Sequence
  Oligonucleotide Detection Strand
gcttgtctacctacgcacacctggag

SEQ ID NO: 251 - DNA, 26 bp, Artificial Sequence
  Oligonucleotide Detection Strand
gcttgtctacctactaacacctggag

SEQ ID NO: 252 - DNA, 26 bp, Artificial Sequence
  Oligonucleotide Detection Strand
gcttgtctacctactcacacctggag

SEQ ID NO: 253 - DNA, 26 bp, Artificial Sequence
  Oligonucleotide Detection Strand
gcttgtctacctacttacacctggag

SEQ ID NO: 254 - DNA, 26 bp, Artificial Sequence
  Oligonucleotide Detection Strand
gcttgtctacctatcaacacctggag

SEQ ID NO: 255 - DNA, 26 bp, Artificial Sequence
  Oligonucleotide Detection Strand
gcttgtctacctatctacacctggag

SEQ ID NO: 256 - DNA, 26 bp, Artificial Sequence
  Oligonucleotide Detection Strand
gcttgtctacctcaaaacacctggag

SEQ ID NO: 257 - DNA, 26 bp, Artificial Sequence
  Oligonucleotide Detection Strand
gcttgtctacctcaacacacctggag

SEQ ID NO: 258 - DNA, 26 bp, Artificial Sequence
  Oligonucleotide Detection Strand
gcttgtctacctcaatacacctggag

SEQ ID NO: 259 - DNA, 26 bp, Artificial Sequence
  Oligonucleotide Detection Strand
gcttgtctacctcaccacacctggag

SEQ ID NO: 260 - DNA, 26 bp, Artificial Sequence
  Oligonucleotide Detection Strand
gcttgtctacctcactacacctggag

SEQ ID NO: 261 - DNA, 26 bp, Artificial Sequence
  Oligonucleotide Detection Strand
gcttgtctacctcataacacctggag

SEQ ID NO: 262 - DNA, 26 bp, Artificial Sequence
  Oligonucleotide Detection Strand
gcttgtctacctcatcacacctggag

SEQ ID NO: 263 - DNA, 26 bp, Artificial Sequence
  Oligonucleotide Detection Strand
gcttgtctacctcattacacctggag

SEQ ID NO: 264 - DNA, 26 bp, Artificial Sequence
  Oligonucleotide Detection Strand
gcttgtctacctcgcaacacctggag

SEQ ID NO: 265 - DNA, 26 bp, Artificial Sequence
  Oligonucleotide Detection Strand
gcttgtctacctcgctacacctggag

SEQ ID NO: 266 - DNA, 26 bp, Artificial Sequence
  Oligonucleotide Detection Strand
gcttgtctacctctaaacacctggag

SEQ ID NO: 267 - DNA, 26 bp, Artificial Sequence
  Oligonucleotide Detection Strand
gcttgtctacctctacacacctggag

SEQ ID NO: 268 - DNA, 26 bp, Artificial Sequence
  Oligonucleotide Detection Strand
gcttgtctacctctcaacacctggag

SEQ ID NO: 269 - DNA, 26 bp, Artificial Sequence
  Oligonucleotide Detection Strand
gcttgtctacctctctacacctggag

SEQ ID NO: 270 - DNA, 26 bp, Artificial Sequence
  Oligonucleotide Detection Strand
gcttgtctacctcttcacacctggag

SEQ ID NO: 271 - DNA, 26 bp, Artificial Sequence
  Oligonucleotide Detection Strand
gcttgtctacctgccaacacctggag

SEQ ID NO: 272 - DNA, 26 bp, Artificial Sequence
  Oligonucleotide Detection Strand
gcttgtctacctgcctacacctggag

SEQ ID NO: 273 - DNA, 26 bp, Artificial Sequence
  Oligonucleotide Detection Strand
gcttgtctacctgctaacacctggag

SEQ ID NO: 274 - DNA, 26 bp, Artificial Sequence
  Oligonucleotide Detection Strand
gcttgtctacctgcttacacctggag

SEQ ID NO: 275 - DNA, 26 bp, Artificial Sequence
  Oligonucleotide Detection Strand
gcttgtctacctggaaacacctggag

SEQ ID NO: 276 - DNA, 26 bp, Artificial Sequence
  Oligonucleotide Detection Strand
gcttgtctacctgtcaacacctggag

SEQ ID NO: 277 - DNA, 26 bp, Artificial Sequence
  Oligonucleotide Detection Strand
gcttgtctacctgtctacacctggag

SEQ ID NO: 278 - DNA, 26 bp, Artificial Sequence
  Oligonucleotide Detection Strand
gcttgtctaccttaccacacctggag

SEQ ID NO: 279 - DNA, 26 bp, Artificial Sequence
  Oligonucleotide Detection Strand
gcttgtctaccttactacacctggag

SEQ ID NO: 280 - DNA, 26 bp, Artificial Sequence
  Oligonucleotide Detection Strand
gcttgtctaccttcaaacacctggag

SEQ ID NO: 281 - DNA, 26 bp, Artificial Sequence
  Oligonucleotide Detection Strand
gcttgtctaccttcacacacctggag

SEQ ID NO: 282 - DNA, 26 bp, Artificial Sequence
  Oligonucleotide Detection Strand
gcttgtctaccttcatacacctggag

SEQ ID NO: 283 - DNA, 26 bp, Artificial Sequence
  Oligonucleotide Detection Strand
gcttgtctaccttctaacacctggag

SEQ ID NO: 284 - DNA, 26 bp, Artificial Sequence
  Oligonucleotide Detection Strand
gcttgtctaccttctcacacctggag

SEQ ID NO: 285 - DNA, 26 bp, Artificial Sequence
  Oligonucleotide Detection Strand
gcttgtctaccttcttacacctggag

SEQ ID NO: 286 - DNA, 26 bp, Artificial Sequence
  Oligonucleotide Detection Strand
gcttgtctaccttgtcacacctggag

SEQ ID NO: 287 - DNA, 26 bp, Artificial Sequence
  Oligonucleotide Detection Strand
gcttgtctacgaaaacacacctggag

SEQ ID NO: 288 - DNA, 26 bp, Artificial Sequence
  Oligonucleotide Detection Strand
gcttgtctacgaaaccacacctggag

SEQ ID NO: 289 - DNA, 26 bp, Artificial Sequence
  Oligonucleotide Detection Strand
gcttgtctacgaaactacacctggag

SEQ ID NO: 290 - DNA, 26 bp, Artificial Sequence
  Oligonucleotide Detection Strand
gcttgtctacgaaatcacacctggag

SEQ ID NO: 291 - DNA, 26 bp, Artificial Sequence
  Oligonucleotide Detection Strand
gcttgtctacgaacacacacctggag

SEQ ID NO: 292 - DNA, 26 bp, Artificial Sequence
  Oligonucleotide Detection Strand
gcttgtctacgaacatacacctggag

SEQ ID NO: 293 - DNA, 26 bp, Artificial Sequence
  Oligonucleotide Detection Strand
gcttgtctacgaaccaacacctggag

SEQ ID NO: 294 - DNA, 26 bp, Artificial Sequence
  Oligonucleotide Detection Strand
gcttgtctacgaacccacacctggag

SEQ ID NO: 295 - DNA, 26 bp, Artificial Sequence
  Oligonucleotide Detection Strand
gcttgtctacgaacctacacctggag

SEQ ID NO: 296 - DNA, 26 bp, Artificial Sequence
  Oligonucleotide Detection Strand
gcttgtctacgaacgaacacctggag

SEQ ID NO: 297 - DNA, 26 bp, Artificial Sequence
  Oligonucleotide Detection Strand
gcttgtctacgaacgcacacctggag

SEQ ID NO: 298 - DNA, 26 bp, Artificial Sequence
  Oligonucleotide Detection Strand
gcttgtctacgaactaacacctggag

SEQ ID NO: 299 - DNA, 26 bp, Artificial Sequence
  Oligonucleotide Detection Strand
gcttgtctacgaactcacacctggag

SEQ ID NO: 300 - DNA, 26 bp, Artificial Sequence
  Oligonucleotide Detection Strand
gcttgtctacgaacttacacctggag

SEQ ID NO: 301 - DNA, 26 bp, Artificial Sequence
  Oligonucleotide Detection Strand
gcttgtctacgaatacacacctggag

SEQ ID NO: 302 - DNA, 26 bp, Artificial Sequence
  Oligonucleotide Detection Strand
gcttgtctacgaatcaacacctggag

SEQ ID NO: 303 - DNA, 26 bp, Artificial Sequence
  Oligonucleotide Detection Strand
gcttgtctacgaatctacacctggag

SEQ ID NO: 304 - DNA, 26 bp, Artificial Sequence
  Oligonucleotide Detection Strand
gcttgtctacgaccaaacacctggag

SEQ ID NO: 305 - DNA, 26 bp, Artificial Sequence
  Oligonucleotide Detection Strand
gcttgtctacgaccacacacctggag

SEQ ID NO: 306 - DNA, 26 bp, Artificial Sequence
  Oligonucleotide Detection Strand
gcttgtctacgaccatacacctggag

SEQ ID NO: 307 - DNA, 26 bp, Artificial Sequence
  Oligonucleotide Detection Strand
gcttgtctacgacccaacacctggag

SEQ ID NO: 308 - DNA, 26 bp, Artificial Sequence
  Oligonucleotide Detection Strand
gcttgtctacgaccctacacctggag

SEQ ID NO: 309 - DNA, 26 bp, Artificial Sequence
  Oligonucleotide Detection Strand
gcttgtctacgaccgaacacctggag

SEQ ID NO: 310 - DNA, 26 bp, Artificial Sequence
  Oligonucleotide Detection Strand
gcttgtctacgacctaacacctggag

SEQ ID NO: 311 - DNA, 26 bp, Artificial Sequence
  Oligonucleotide Detection Strand
gcttgtctacgacctcacacctggag

SEQ ID NO: 312 - DNA, 26 bp, Artificial Sequence
  Oligonucleotide Detection Strand
gcttgtctacgaccttacacctggag

SEQ ID NO: 313 - DNA, 26 bp, Artificial Sequence
  Oligonucleotide Detection Strand
gcttgtctacgacgaaacacctggag

SEQ ID NO: 314 - DNA, 26 bp, Artificial Sequence
  Oligonucleotide Detection Strand
gcttgtctacgacgcaacacctggag

SEQ ID NO: 315 - DNA, 26 bp, Artificial Sequence
  Oligonucleotide Detection Strand
gcttgtctacgacgctacacctggag

SEQ ID NO: 316 - DNA, 26 bp, Artificial Sequence
  Oligonucleotide Detection Strand
gcttgtctacgacggaacacctggag

SEQ ID NO: 317 - DNA, 26 bp, Artificial Sequence
  Oligonucleotide Detection Strand
gcttgtctacgactaaacacctggag

SEQ ID NO: 318 - DNA, 26 bp, Artificial Sequence
  Oligonucleotide Detection Strand
gcttgtctacgactacacacctggag

SEQ ID NO: 319 - DNA, 26 bp, Artificial Sequence
  Oligonucleotide Detection Strand
gcttgtctacgactcaacacctggag

SEQ ID NO: 320 - DNA, 26 bp, Artificial Sequence
  Oligonucleotide Detection Strand
gcttgtctacgactctacacctggag

SEQ ID NO: 321 - DNA, 26 bp, Artificial Sequence
  Oligonucleotide Detection Strand
gcttgtctacgacttcacacctggag

SEQ ID NO: 322 - DNA, 26 bp, Artificial Sequence
  Oligonucleotide Detection Strand
gcttgtctacgcaaaaacacctggag

SEQ ID NO: 323 - DNA, 26 bp, Artificial Sequence
  Oligonucleotide Detection Strand
gcttgtctacgcaaacacacctggag

SEQ ID NO: 324 - DNA, 26 bp, Artificial Sequence
  Oligonucleotide Detection Strand
gcttgtctacgcaaatacacctggag

SEQ ID NO: 325 - DNA, 26 bp, Artificial Sequence
  Oligonucleotide Detection Strand
gcttgtctacgcaaccacacctggag

SEQ ID NO: 326 - DNA, 26 bp, Artificial Sequence
  Oligonucleotide Detection Strand
gcttgtctacgcaactacacctggag

SEQ ID NO: 327 - DNA, 26 bp, Artificial Sequence
  Oligonucleotide Detection Strand
gcttgtctacgcaataacacctggag

SEQ ID NO: 328 - DNA, 26 bp, Artificial Sequence
  Oligonucleotide Detection Strand
gcttgtctacgcaatcacacctggag

SEQ ID NO: 329 - DNA, 26 bp, Artificial Sequence
  Oligonucleotide Detection Strand
gcttgtctacgcacacacacctggag

SEQ ID NO: 330 - DNA, 26 bp, Artificial Sequence
  Oligonucleotide Detection Strand
gcttgtctacgcacatacacctggag

SEQ ID NO: 331 - DNA, 26 bp, Artificial Sequence
  Oligonucleotide Detection Strand
gcttgtctacgcaccaacacctggag

SEQ ID NO: 332 - DNA, 26 bp, Artificial Sequence
  Oligonucleotide Detection Strand
gcttgtctacgcacctacacctggag

SEQ ID NO: 333 - DNA, 26 bp, Artificial Sequence
  Oligonucleotide Detection Strand
gcttgtctacgcacgaacacctggag

SEQ ID NO: 334 - DNA, 26 bp, Artificial Sequence
  Oligonucleotide Detection Strand
gcttgtctacgcactaacacctggag

SEQ ID NO: 335 - DNA, 26 bp, Artificial Sequence
  Oligonucleotide Detection Strand
gcttgtctacgcactcacacctggag

SEQ ID NO: 336 - DNA, 26 bp, Artificial Sequence
  Oligonucleotide Detection Strand
gcttgtctacgcacttacacctggag

SEQ ID NO: 337 - DNA, 26 bp, Artificial Sequence
  Oligonucleotide Detection Strand
gcttgtctacgcataaacacctggag

SEQ ID NO: 338 - DNA, 26 bp, Artificial Sequence
  Oligonucleotide Detection Strand
gcttgtctacgcatacacacctggag

SEQ ID NO: 339 - DNA, 26 bp, Artificial Sequence
  Oligonucleotide Detection Strand
gcttgtctacgcatcaacacctggag

SEQ ID NO: 340 - DNA, 26 bp, Artificial Sequence
  Oligonucleotide Detection Strand
gcttgtctacgcatctacacctggag

SEQ ID NO: 341 - DNA, 26 bp, Artificial Sequence
  Oligonucleotide Detection Strand
gcttgtctacgccaaaacacctggag

SEQ ID NO: 342 - DNA, 26 bp, Artificial Sequence
  Oligonucleotide Detection Strand
gcttgtctacgccaacacacctggag

SEQ ID NO: 343 - DNA, 26 bp, Artificial Sequence
  Oligonucleotide Detection Strand
gcttgtctacgccaatacacctggag

SEQ ID NO: 344 - DNA, 26 bp, Artificial Sequence
  Oligonucleotide Detection Strand
gcttgtctacgccactacacctggag

SEQ ID NO: 345 - DNA, 26 bp, Artificial Sequence
  Oligonucleotide Detection Strand
gcttgtctacgccataacacctggag

SEQ ID NO: 346 - DNA, 26 bp, Artificial Sequence
  Oligonucleotide Detection Strand
gcttgtctacgccatcacacctggag

SEQ ID NO: 347 - DNA, 26 bp, Artificial Sequence
  Oligonucleotide Detection Strand
gcttgtctacgccattacacctggag

SEQ ID NO: 348 - DNA, 26 bp, Artificial Sequence
  Oligonucleotide Detection Strand
gcttgtctacgcccaaacacctggag

SEQ ID NO: 349 - DNA, 26 bp, Artificial Sequence
  Oligonucleotide Detection Strand
gcttgtctacgcccatacacctggag

SEQ ID NO: 350 - DNA, 26 bp, Artificial Sequence
  Oligonucleotide Detection Strand
gcttgtctacgccctaacacctggag

SEQ ID NO: 351 - DNA, 26 bp, Artificial Sequence
  Oligonucleotide Detection Strand
gcttgtctacgcccttacacctggag

SEQ ID NO: 352 - DNA, 26 bp, Artificial Sequence
  Oligonucleotide Detection Strand
gcttgtctacgcctaaacacctggag

SEQ ID NO: 353 - DNA, 26 bp, Artificial Sequence
  Oligonucleotide Detection Strand
gcttgtctacgcctacacacctggag

SEQ ID NO: 354 - DNA, 26 bp, Artificial Sequence
  Oligonucleotide Detection Strand
gcttgtctacgcctcaacacctggag

SEQ ID NO: 355 - DNA, 26 bp, Artificial Sequence
  Oligonucleotide Detection Strand
gcttgtctacgcctctacacctggag

SEQ ID NO: 356 - DNA, 26 bp, Artificial Sequence
  Oligonucleotide Detection Strand
gcttgtctacgccttcacacctggag

SEQ ID NO: 357 - DNA, 26 bp, Artificial Sequence
  Oligonucleotide Detection Strand
gcttgtctacgctaaaacacctggag

SEQ ID NO: 358 - DNA, 26 bp, Artificial Sequence
  Oligonucleotide Detection Strand
gcttgtctacgctaacacacctggag

SEQ ID NO: 359 - DNA, 26 bp, Artificial Sequence
  Oligonucleotide Detection Strand
gcttgtctacgctaccacacctggag

SEQ ID NO: 360 - DNA, 26 bp, Artificial Sequence
  Oligonucleotide Detection Strand
gcttgtctacgctactacacctggag

SEQ ID NO: 361 - DNA, 26 bp, Artificial Sequence
  Oligonucleotide Detection Strand
gcttgtctacgctatcacacctggag

SEQ ID NO: 362 - DNA, 26 bp, Artificial Sequence
  Oligonucleotide Detection Strand
gcttgtctacgctcaaacacctggag

SEQ ID NO: 363 - DNA, 26 bp, Artificial Sequence
  Oligonucleotide Detection Strand
gcttgtctacgctcacacacctggag

SEQ ID NO: 364 - DNA, 26 bp, Artificial Sequence
  Oligonucleotide Detection Strand
gcttgtctacgctcatacacctggag

SEQ ID NO: 365 - DNA, 26 bp, Artificial Sequence
  Oligonucleotide Detection Strand
gcttgtctacgctctaacacctggag

SEQ ID NO: 366 - DNA, 26 bp, Artificial Sequence
  Oligonucleotide Detection Strand
gcttgtctacgctctcacacctggag

SEQ ID NO: 367 - DNA, 26 bp, Artificial Sequence
  Oligonucleotide Detection Strand
gcttgtctacgctcttacacctggag

SEQ ID NO: 368 - DNA, 26 bp, Artificial Sequence
  Oligonucleotide Detection Strand
gcttgtctacgctgctacacctggag

SEQ ID NO: 369 - DNA, 26 bp, Artificial Sequence
  Oligonucleotide Detection Strand
gcttgtctacgctggaacacctggag

SEQ ID NO: 370 - DNA, 26 bp, Artificial Sequence
  Oligonucleotide Detection Strand
gcttgtctacgctgtcacacctggag

SEQ ID NO: 371 - DNA, 26 bp, Artificial Sequence
  Oligonucleotide Detection Strand
gcttgtctacgcttacacacctggag

SEQ ID NO: 372 - DNA, 26 bp, Artificial Sequence
  Oligonucleotide Detection Strand
gcttgtctacgcttcaacacctggag

SEQ ID NO: 373 - DNA, 26 bp, Artificial Sequence
  Oligonucleotide Detection Strand
gcttgtctacgcttctacacctggag

SEQ ID NO: 374 - DNA, 26 bp, Artificial Sequence
  Oligonucleotide Detection Strand
gcttgtctacggaaaaacacctggag

SEQ ID NO: 375 - DNA, 26 bp, Artificial Sequence
  Oligonucleotide Detection Strand
gcttgtctacggaaacacacctggag

SEQ ID NO: 376 - DNA, 26 bp, Artificial Sequence
  Oligonucleotide Detection Strand
gcttgtctacggaaatacacctggag

SEQ ID NO: 377 - DNA, 26 bp, Artificial Sequence
  Oligonucleotide Detection Strand
gcttgtctacggaaccacacctggag

SEQ ID NO: 378 - DNA, 26 bp, Artificial Sequence
  Oligonucleotide Detection Strand
gcttgtctacggaactacacctggag

SEQ ID NO: 379 - DNA, 26 bp, Artificial Sequence
  Oligonucleotide Detection Strand
gcttgtctacggaataacacctggag

SEQ ID NO: 380 - DNA, 26 bp, Artificial Sequence
  Oligonucleotide Detection Strand
gcttgtctacggaatcacacctggag

SEQ ID NO: 381 - DNA, 26 bp, Artificial Sequence
  Oligonucleotide Detection Strand
gcttgtctacggaccaacacctggag

SEQ ID NO: 382 - DNA, 26 bp, Artificial Sequence
  Oligonucleotide Detection Strand
gcttgtctacggacctacacctggag

SEQ ID NO: 383 - DNA, 26 bp, Artificial Sequence
  Oligonucleotide Detection Strand
gcttgtctacggacgaacacctggag

SEQ ID NO: 384 - DNA, 26 bp, Artificial Sequence
  Oligonucleotide Detection Strand
gcttgtctacggactaacacctggag

SEQ ID NO: 385 - DNA, 26 bp, Artificial Sequence
  Oligonucleotide Detection Strand
gcttgtctacggactcacacctggag

SEQ ID NO: 386 - DNA, 26 bp, Artificial Sequence
  Oligonucleotide Detection Strand
gcttgtctacggacttacacctggag

SEQ ID NO: 387 - DNA, 26 bp, Artificial Sequence
  Oligonucleotide Detection Strand
gcttgtctacggctaaacacctggag

SEQ ID NO: 388 - DNA, 26 bp, Artificial Sequence
  Oligonucleotide Detection Strand
gcttgtctacggctacacacctggag

SEQ ID NO: 389 - DNA, 26 bp, Artificial Sequence
  Oligonucleotide Detection Strand
gcttgtctacggctcaacacctggag

SEQ ID NO: 390 - DNA, 26 bp, Artificial Sequence
  Oligonucleotide Detection Strand
gcttgtctacggcttcacacctggag

SEQ ID NO: 391 - DNA, 26 bp, Artificial Sequence
  Oligonucleotide Detection Strand
gcttgtctactaaaccacacctggag

SEQ ID NO: 392 - DNA, 26 bp, Artificial Sequence
  Oligonucleotide Detection Strand
gcttgtctactaacacacacctggag

SEQ ID NO: 393 - DNA, 26 bp, Artificial Sequence
  Oligonucleotide Detection Strand
gcttgtctactaaccaacacctggag

SEQ ID NO: 394 - DNA, 26 bp, Artificial Sequence
  Oligonucleotide Detection Strand
gcttgtctactaacccacacctggag

SEQ ID NO: 395 - DNA, 26 bp, Artificial Sequence
  Oligonucleotide Detection Strand
gcttgtctactaacctacacctggag

SEQ ID NO: 396 - DNA, 26 bp, Artificial Sequence
  Oligonucleotide Detection Strand
gcttgtctactaacgaacacctggag

SEQ ID NO: 397 - DNA, 26 bp, Artificial Sequence
  Oligonucleotide Detection Strand
gcttgtctactaacgcacacctggag

SEQ ID NO: 398 - DNA, 26 bp, Artificial Sequence
  Oligonucleotide Detection Strand
gcttgtctactaactcacacctggag

SEQ ID NO: 399 - DNA, 26 bp, Artificial Sequence
  Oligonucleotide Detection Strand
gcttgtctactacaccacacctggag

SEQ ID NO: 400 - DNA, 26 bp, Artificial Sequence
  Oligonucleotide Detection Strand
gcttgtctactacactacacctggag

SEQ ID NO: 401 - DNA, 26 bp, Artificial Sequence
  Oligonucleotide Detection Strand
gcttgtctactacatcacacctggag

SEQ ID NO: 402 - DNA, 26 bp, Artificial Sequence
  Oligonucleotide Detection Strand
gcttgtctactaccaaacacctggag

SEQ ID NO: 403 - DNA, 26 bp, Artificial Sequence
  Oligonucleotide Detection Strand
gcttgtctactaccacacacctggag

SEQ ID NO: 404 - DNA, 26 bp, Artificial Sequence
  Oligonucleotide Detection Strand
gcttgtctactaccatacacctggag

SEQ ID NO: 405 - DNA, 26 bp, Artificial Sequence
  Oligonucleotide Detection Strand
gcttgtctactacccaacacctggag

SEQ ID NO: 406 - DNA, 26 bp, Artificial Sequence
  Oligonucleotide Detection Strand
gcttgtctactaccccacacctggag

SEQ ID NO: 407 - DNA, 26 bp, Artificial Sequence
  Oligonucleotide Detection Strand
gcttgtctactaccctacacctggag

SEQ ID NO: 408 - DNA, 26 bp, Artificial Sequence
  Oligonucleotide Detection Strand
gcttgtctactaccgaacacctggag

SEQ ID NO: 409 - DNA, 26 bp, Artificial Sequence
  Oligonucleotide Detection Strand
gcttgtctactaccgcacacctggag

SEQ ID NO: 410 - DNA, 26 bp, Artificial Sequence
  Oligonucleotide Detection Strand
gcttgtctactacctaacacctggag

SEQ ID NO: 411 - DNA, 26 bp, Artificial Sequence
  Oligonucleotide Detection Strand
gcttgtctactacctcacacctggag

SEQ ID NO: 412 - DNA, 26 bp, Artificial Sequence
  Oligonucleotide Detection Strand
gcttgtctactaccttacacctggag

SEQ ID NO: 413 - DNA, 26 bp, Artificial Sequence
  Oligonucleotide Detection Strand
gcttgtctactacgaaacacctggag

SEQ ID NO: 414 - DNA, 26 bp, Artificial Sequence
  Oligonucleotide Detection Strand
gcttgtctactacgcaacacctggag

SEQ ID NO: 415 - DNA, 26 bp, Artificial Sequence
  Oligonucleotide Detection Strand
gcttgtctactacgccacacctggag

SEQ ID NO: 416 - DNA, 26 bp, Artificial Sequence
  Oligonucleotide Detection Strand
gcttgtctactacgctacacctggag

SEQ ID NO: 417 - DNA, 26 bp, Artificial Sequence
  Oligonucleotide Detection Strand
gcttgtctactacggaacacctggag

SEQ ID NO: 418 - DNA, 26 bp, Artificial Sequence
  Oligonucleotide Detection Strand
gcttgtctactactacacacctggag

SEQ ID NO: 419 - DNA, 26 bp, Artificial Sequence
  Oligonucleotide Detection Strand
gcttgtctactactcaacacctggag

SEQ ID NO: 420 - DNA, 26 bp, Artificial Sequence
  Oligonucleotide Detection Strand
gcttgtctactactctacacctggag

SEQ ID NO: 421 - DNA, 26 bp, Artificial Sequence
  Oligonucleotide Detection Strand
gcttgtctactacttcacacctggag

SEQ ID NO: 422 - DNA, 26 bp, Artificial Sequence
  Oligonucleotide Detection Strand
gcttgtctactatcacacacctggag

SEQ ID NO: 423 - DNA, 26 bp, Artificial Sequence
  Oligonucleotide Detection Strand
gcttgtctactatcccacacctggag

SEQ ID NO: 424 - DNA, 26 bp, Artificial Sequence
  Oligonucleotide Detection Strand
gcttgtctactatcctacacctggag

SEQ ID NO: 425 - DNA, 26 bp, Artificial Sequence
  Oligonucleotide Detection Strand
gcttgtctactatcgcacacctggag

SEQ ID NO: 426 - DNA, 26 bp, Artificial Sequence
  Oligonucleotide Detection Strand
gcttgtctactatctcacacctggag

SEQ ID NO: 427 - DNA, 26 bp, Artificial Sequence
  Oligonucleotide Detection Strand
gcttgtctactcaaacacacctggag

SEQ ID NO: 428 - DNA, 26 bp, Artificial Sequence
  Oligonucleotide Detection Strand
gcttgtctactcaaccacacctggag

SEQ ID NO: 429 - DNA, 26 bp, Artificial Sequence
  Oligonucleotide Detection Strand
gcttgtctactcaactacacctggag

SEQ ID NO: 430 - DNA, 26 bp, Artificial Sequence
  Oligonucleotide Detection Strand
gcttgtctactcaatcacacctggag

SEQ ID NO: 431 - DNA, 26 bp, Artificial Sequence
  Oligonucleotide Detection Strand
gcttgtctactcacacacacctggag

SEQ ID NO: 432 - DNA, 26 bp, Artificial Sequence
  Oligonucleotide Detection Strand
gcttgtctactcacatacacctggag

SEQ ID NO: 433 - DNA, 26 bp, Artificial Sequence
  Oligonucleotide Detection Strand
gcttgtctactcaccaacacctggag

SEQ ID NO: 434 - DNA, 26 bp, Artificial Sequence
  Oligonucleotide Detection Strand
gcttgtctactcacccacacctggag

SEQ ID NO: 435 - DNA, 26 bp, Artificial Sequence
  Oligonucleotide Detection Strand
gcttgtctactcacctacacctggag

SEQ ID NO: 436 - DNA, 26 bp, Artificial Sequence
  Oligonucleotide Detection Strand
gcttgtctactcacgaacacctggag

SEQ ID NO: 437 - DNA, 26 bp, Artificial Sequence
  Oligonucleotide Detection Strand
gcttgtctactcacgcacacctggag

SEQ ID NO: 438 - DNA, 26 bp, Artificial Sequence
  Oligonucleotide Detection Strand
gcttgtctactcactaacacctggag

SEQ ID NO: 439 - DNA, 26 bp, Artificial Sequence
  Oligonucleotide Detection Strand
gcttgtctactcactcacacctggag

SEQ ID NO: 440 - DNA, 26 bp, Artificial Sequence
  Oligonucleotide Detection Strand
gcttgtctactcacttacacctggag

SEQ ID NO: 441 - DNA, 26 bp, Artificial Sequence
  Oligonucleotide Detection Strand
gcttgtctactcatacacacctggag

SEQ ID NO: 442 - DNA, 26 bp, Artificial Sequence
  Oligonucleotide Detection Strand
gcttgtctactcatcaacacctggag

SEQ ID NO: 443 - DNA, 26 bp, Artificial Sequence
  Oligonucleotide Detection Strand
gcttgtctactcatctacacctggag

SEQ ID NO: 444 - DNA, 26 bp, Artificial Sequence
  Oligonucleotide Detection Strand
gcttgtctactcgcaaacacctggag

SEQ ID NO: 445 - DNA, 26 bp, Artificial Sequence
  Oligonucleotide Detection Strand
gcttgtctactcgcacacacctggag

SEQ ID NO: 446 - DNA, 26 bp, Artificial Sequence
  Oligonucleotide Detection Strand
gcttgtctactcgcatacacctggag

SEQ ID NO: 447 - DNA, 26 bp, Artificial Sequence
  Oligonucleotide Detection Strand
gcttgtctactcgccaacacctggag

SEQ ID NO: 448 - DNA, 26 bp, Artificial Sequence
  Oligonucleotide Detection Strand
gcttgtctactcgcctacacctggag

SEQ ID NO: 449 - DNA, 26 bp, Artificial Sequence
  Oligonucleotide Detection Strand
gcttgtctactcgctaacacctggag

SEQ ID NO: 450 - DNA, 26 bp, Artificial Sequence
  Oligonucleotide Detection Strand
gcttgtctactcgctcacacctggag

SEQ ID NO: 451 - DNA, 26 bp, Artificial Sequence
  Oligonucleotide Detection Strand
gcttgtctactcgcttacacctggag

SEQ ID NO: 452 - DNA, 26 bp, Artificial Sequence
  Oligonucleotide Detection Strand
gcttgtctactctaacacacctggag

SEQ ID NO: 453 - DNA, 26 bp, Artificial Sequence
  Oligonucleotide Detection Strand
gcttgtctactctaccacacctggag

SEQ ID NO: 454 - DNA, 26 bp, Artificial Sequence
  Oligonucleotide Detection Strand
gcttgtctactctactacacctggag

SEQ ID NO: 455 - DNA, 26 bp, Artificial Sequence
  Oligonucleotide Detection Strand
gcttgtctactctatcacacctggag

SEQ ID NO: 456 - DNA, 26 bp, Artificial Sequence
  Oligonucleotide Detection Strand
gcttgtctactctcaaacacctggag

SEQ ID NO: 457 - DNA, 26 bp, Artificial Sequence
  Oligonucleotide Detection Strand
gcttgtctactctcacacacctggag

SEQ ID NO: 458 - DNA, 26 bp, Artificial Sequence
  Oligonucleotide Detection Strand
gcttgtctactctcatacacctggag

SEQ ID NO: 459 - DNA, 26 bp, Artificial Sequence
  Oligonucleotide Detection Strand
gcttgtctactctcgcacacctggag

SEQ ID NO: 460 - DNA, 26 bp, Artificial Sequence
  Oligonucleotide Detection Strand
gcttgtctactctctaacacctggag

SEQ ID NO: 461 - DNA, 26 bp, Artificial Sequence
  Oligonucleotide Detection Strand
gcttgtctactctctcacacctggag

SEQ ID NO: 462 - DNA, 26 bp, Artificial Sequence
  Oligonucleotide Detection Strand
gcttgtctactctcttacacctggag

SEQ ID NO: 463 - DNA, 26 bp, Artificial Sequence
  Oligonucleotide Detection Strand
gcttgtctactctgccacacctggag

SEQ ID NO: 464 - DNA, 26 bp, Artificial Sequence
  Oligonucleotide Detection Strand
gcttgtctactctgctacacctggag

SEQ ID NO: 465 - DNA, 26 bp, Artificial Sequence
  Oligonucleotide Detection Strand
gcttgtctactctggaacacctggag

SEQ ID NO: 466 - DNA, 26 bp, Artificial Sequence
  Oligonucleotide Detection Strand
gcttgtctactctgtcacacctggag

SEQ ID NO: 467 - DNA, 26 bp, Artificial Sequence
  Oligonucleotide Detection Strand
gcttgtctactcttacacacctggag

SEQ ID NO: 468 - DNA, 26 bp, Artificial Sequence
  Oligonucleotide Detection Strand
gcttgtctactcttcaacacctggag

SEQ ID NO: 469 - DNA, 26 bp, Artificial Sequence
  Oligonucleotide Detection Strand
gcttgtctactcttctacacctggag

SEQ ID NO: 470 - DNA, 26 bp, Artificial Sequence
  Oligonucleotide Detection Strand
gcttgtctactgccaaacacctggag

SEQ ID NO: 471 - DNA, 26 bp, Artificial Sequence
  Oligonucleotide Detection Strand
gcttgtctactgccacacacctggag

SEQ ID NO: 472 - DNA, 26 bp, Artificial Sequence
  Oligonucleotide Detection Strand
gcttgtctactgccatacacctggag

SEQ ID NO: 473 - DNA, 26 bp, Artificial Sequence
  Oligonucleotide Detection Strand
gcttgtctactgcccaacacctggag

SEQ ID NO: 474 - DNA, 26 bp, Artificial Sequence
  Oligonucleotide Detection Strand
gcttgtctactgccctacacctggag

SEQ ID NO: 475 - DNA, 26 bp, Artificial Sequence
  Oligonucleotide Detection Strand
gcttgtctactgcctaacacctggag

SEQ ID NO: 476 - DNA, 26 bp, Artificial Sequence
  Oligonucleotide Detection Strand
gcttgtctactgcctcacacctggag

SEQ ID NO: 477 - DNA, 26 bp, Artificial Sequence
  Oligonucleotide Detection Strand
gcttgtctactgccttacacctggag

SEQ ID NO: 478 - DNA, 26 bp, Artificial Sequence
  Oligonucleotide Detection Strand
gcttgtctactgctaaacacctggag

SEQ ID NO: 479 - DNA, 26 bp, Artificial Sequence
  Oligonucleotide Detection Strand
gcttgtctactgctacacacctggag

SEQ ID NO: 480 - DNA, 26 bp, Artificial Sequence
  Oligonucleotide Detection Strand
gcttgtctactgctcaacacctggag

SEQ ID NO: 481 - DNA, 26 bp, Artificial Sequence
  Oligonucleotide Detection Strand
gcttgtctactgctctacacctggag

SEQ ID NO: 482 - DNA, 26 bp, Artificial Sequence
  Oligonucleotide Detection Strand
gcttgtctactgcttcacacctggag

SEQ ID NO: 483 - DNA, 26 bp, Artificial Sequence
  Oligonucleotide Detection Strand
gcttgtctactggaaaacacctggag

SEQ ID NO: 484 - DNA, 26 bp, Artificial Sequence
  Oligonucleotide Detection Strand
gcttgtctactggaacacacctggag

SEQ ID NO: 485 - DNA, 26 bp, Artificial Sequence
  Oligonucleotide Detection Strand
gcttgtctactggaatacacctggag

SEQ ID NO: 486 - DNA, 26 bp, Artificial Sequence
  Oligonucleotide Detection Strand
gcttgtctactggaccacacctggag

SEQ ID NO: 487 - DNA, 26 bp, Artificial Sequence
  Oligonucleotide Detection Strand
gcttgtctactggactacacctggag

SEQ ID NO: 488 - DNA, 26 bp, Artificial Sequence
  Oligonucleotide Detection Strand
gcttgtctactgtcaaacacctggag

SEQ ID NO: 489 - DNA, 26 bp, Artificial Sequence
  Oligonucleotide Detection Strand
gcttgtctactgtcacacacctggag

SEQ ID NO: 490 - DNA, 26 bp, Artificial Sequence
  Oligonucleotide Detection Strand
gcttgtctactgtcatacacctggag

SEQ ID NO: 491 - DNA, 26 bp, Artificial Sequence
  Oligonucleotide Detection Strand
gcttgtctactgtctaacacctggag

SEQ ID NO: 492 - DNA, 26 bp, Artificial Sequence
  Oligonucleotide Detection Strand
gcttgtctactgtctcacacctggag

SEQ ID NO: 493 - DNA, 26 bp, Artificial Sequence
  Oligonucleotide Detection Strand
gcttgtctactgtcttacacctggag

SEQ ID NO: 494 - DNA, 26 bp, Artificial Sequence
  Oligonucleotide Detection Strand
gcttgtctacttacacacacctggag

SEQ ID NO: 495 - DNA, 26 bp, Artificial Sequence
  Oligonucleotide Detection Strand
gcttgtctacttaccaacacctggag

SEQ ID NO: 496 - DNA, 26 bp, Artificial Sequence
  Oligonucleotide Detection Strand
gcttgtctacttacccacacctggag

SEQ ID NO: 497 - DNA, 26 bp, Artificial Sequence
  Oligonucleotide Detection Strand
gcttgtctacttacctacacctggag

SEQ ID NO: 498 - DNA, 26 bp, Artificial Sequence
  Oligonucleotide Detection Strand
gcttgtctacttacgaacacctggag

SEQ ID NO: 499 - DNA, 26 bp, Artificial Sequence
  Oligonucleotide Detection Strand
gcttgtctacttacgcacacctggag

SEQ ID NO: 500 - DNA, 26 bp, Artificial Sequence
  Oligonucleotide Detection Strand
gcttgtctacttactcacacctggag

SEQ ID NO: 501 - DNA, 26 bp, Artificial Sequence
  Oligonucleotide Detection Strand
gcttgtctacttcaacacacctggag

SEQ ID NO: 502 - DNA, 26 bp, Artificial Sequence
  Oligonucleotide Detection Strand
gcttgtctacttcaccacacctggag

SEQ ID NO: 503 - DNA, 26 bp, Artificial Sequence
  Oligonucleotide Detection Strand
gcttgtctacttcactacacctggag

SEQ ID NO: 504 - DNA, 26 bp, Artificial Sequence
  Oligonucleotide Detection Strand
gcttgtctacttcatcacacctggag

SEQ ID NO: 505 - DNA, 26 bp, Artificial Sequence
  Oligonucleotide Detection Strand
gcttgtctacttctacacacctggag

SEQ ID NO: 506 - DNA, 26 bp, Artificial Sequence
  Oligonucleotide Detection Strand
gcttgtctacttctcaacacctggag

SEQ ID NO: 507 - DNA, 26 bp, Artificial Sequence
  Oligonucleotide Detection Strand
gcttgtctacttctctacacctggag

SEQ ID NO: 508 - DNA, 26 bp, Artificial Sequence
  Oligonucleotide Detection Strand
gcttgtctacttcttcacacctggag

SEQ ID NO: 509 - DNA, 26 bp, Artificial Sequence
  Oligonucleotide Detection Strand
gcttgtctacttgtcaacacctggag

SEQ ID NO: 510 - DNA, 26 bp, Artificial Sequence
  Oligonucleotide Detection Strand
gcttgtctacttgtctacacctggag

SEQ ID NO: 511 - DNA, 48 bp, Artificial Sequence
  Oligonucleotide Detection Strand
cagacgtcacgccaaactcactaccagcactcttccgtccactacaac

SEQ ID NO: 512 - DNA, 48 bp, Artificial Sequence
  Oligonucleotide Detection Strand
cagacgtcaccagaacctcactaccagcactcttccgtccactacaac

SEQ ID NO: 513 - DNA, 48 bp, Artificial Sequence
  Oligonucleotide Detection Strand
cagacgtcacaagcctctcactaccagcactcttccgtccactacaac

SEQ ID NO: 514 - DNA, 48 bp, Artificial Sequence
  Oligonucleotide Detection Strand
cagacgtcactgtcctctcactaccagcactcttccgtccactacaac

SEQ ID NO: 515 - DNA, 59 bp, Artificial Sequence
  Oligonucleotide Detection Strand
ccactacaacacatccctcacccgtaacactccttagcctcaccgcaatcgaattccac

SEQ ID NO: 516 - DNA, 59 bp, Artificial Sequence
  Oligonucleotide Detection Strand
ccactacaacacatccctcaccgtcaacactctacagcctcaccgcaatcgaattccac

SEQ ID NO: 517 - DNA, 59 bp, Artificial Sequence
  Oligonucleotide Detection Strand
ccactacaacacatccctcacctcctacactcgctttcctcacgaccttcgaattccac

SEQ ID NO: 518 - DNA, 59 bp, Artificial Sequence
  Oligonucleotide Detection Strand
ccactacaacacatccctcacctcctacactccctaagctcaccgcaatcgaattccac

SEQ ID NO: 519 - DNA, 59 bp, Artificial Sequence
  Oligonucleotide Detection Strand
ccactacaacacatccctcacctcctacactccctaagctcacctgcatcgaattccac

SEQ ID NO: 520 - DNA, 59 bp, Artificial Sequence
  Oligonucleotide Detection Strand
ccactacaacacatccctcacctcctacactccctaagctcacgctcatcgaattccac

SEQ ID NO: 521 - DNA, 19 bp, Artificial Sequence
  Oligonucleotide Detection Strand
ccactacaacgccaaactc

SEQ ID NO: 522 - DNA, 19 bp, Artificial Sequence
  Oligonucleotide Detection Strand
ccactacaacgagcaactc

SEQ ID NO: 523 - DNA, 19 bp, Artificial Sequence
  Oligonucleotide Detection Strand
ccactacaaccaaccactc

SEQ ID NO: 524 - DNA, 19 bp, Artificial Sequence
  Oligonucleotide Detection Strand
ccactacaactcagcactc

SEQ ID NO: 525 - DNA, 19 bp, Artificial Sequence
  Oligonucleotide Detection Strand
ccactacaacctaggactc

SEQ ID NO: 526 - DNA, 19 bp, Artificial Sequence
  Oligonucleotide Detection Strand
ccactacaacatccacctc

SEQ ID NO: 527 - DNA, 19 bp, Artificial Sequence
  Oligonucleotide Detection Strand
ccactacaactctaccctc

SEQ ID NO: 528 - DNA, 19 bp, Artificial Sequence
  Oligonucleotide Detection Strand
ccactacaactctctgctc

SEQ ID NO: 529 - DNA, 11 bp, Artificial Sequence
  Oligonucleotide Detection Strand
accgtcaacac

SEQ ID NO: 530 - DNA, 11 bp, Artificial Sequence
  Oligonucleotide Detection Strand
accacgaacac

SEQ ID NO: 531 - DNA, 11 bp, Artificial Sequence
  Oligonucleotide Detection Strand
acccgtaacac

SEQ ID NO: 532 - DNA, 11 bp, Artificial Sequence
  Oligonucleotide Detection Strand
acaaccgacac

SEQ ID NO: 533 - DNA, 11 bp, Artificial Sequence
  Oligonucleotide Detection Strand
acgcactacac

SEQ ID NO: 534 - DNA, 11 bp, Artificial Sequence
  Oligonucleotide Detection Strand
acctcctacac

SEQ ID NO: 535 - DNA, 11 bp, Artificial Sequence
  Oligonucleotide Detection Strand
accctgtacac

SEQ ID NO: 536 - DNA, 11 bp, Artificial Sequence
  Oligonucleotide Detection Strand
acgaaacccac

SEQ ID NO: 537 - DNA, 11 bp, Artificial Sequence
  Oligonucleotide Detection Strand
acatgacccac

SEQ ID NO: 538 - DNA, 11 bp, Artificial Sequence
  Oligonucleotide Detection Strand
acttctcccac

SEQ ID NO: 539 - DNA, 11 bp, Artificial Sequence
  Oligonucleotide Detection Strand
acatcgcacac

SEQ ID NO: 540 - DNA, 11 bp, Artificial Sequence
  Oligonucleotide Detection Strand
acactgaccac

SEQ ID NO: 541 - DNA, 11 bp, Artificial Sequence
  Oligonucleotide Detection Strand
tccattccctc

SEQ ID NO: 542 - DNA, 11 bp, Artificial Sequence
  Oligonucleotide Detection Strand
tctacagcctc

SEQ ID NO: 543 - DNA, 11 bp, Artificial Sequence
  Oligonucleotide Detection Strand
tccttagcctc

SEQ ID NO: 544 - DNA, 11 bp, Artificial Sequence
  Oligonucleotide Detection Strand
tctagctcctc

SEQ ID NO: 545 - DNA, 11 bp, Artificial Sequence
  Oligonucleotide Detection Strand
tcagtctcctc

SEQ ID NO: 546 - DNA, 11 bp, Artificial Sequence
  Oligonucleotide Detection Strand
tcaacgtcctc

SEQ ID NO: 547 - DNA, 11 bp, Artificial Sequence
  Oligonucleotide Detection Strand
tcctgttcctc

SEQ ID NO: 548 - DNA, 11 bp, Artificial Sequence
  Oligonucleotide Detection Strand
tcgctttcctc

SEQ ID NO: 549 - DNA, 11 bp, Artificial Sequence
  Oligonucleotide Detection Strand
tccctaagctc

SEQ ID NO: 550 - DNA, 11 bp, Artificial Sequence
  Oligonucleotide Detection Strand
tctaccagctc

SEQ ID NO: 551 - DNA, 11 bp, Artificial Sequence
  Oligonucleotide Detection Strand
tcctctagctc

SEQ ID NO: 552 - DNA, 11 bp, Artificial Sequence
  Oligonucleotide Detection Strand
tctcacacctc

SEQ ID NO: 553 - DNA, 18 bp, Artificial Sequence
  Oligonucleotide Detection Strand
acctaacgcgaattccac

SEQ ID NO: 554 - DNA, 18 bp, Artificial Sequence
  Oligonucleotide Detection Strand
accacatgcgaattccac

SEQ ID NO: 555 - DNA, 18 bp, Artificial Sequence
  Oligonucleotide Detection Strand
accgcaatcgaattccac

SEQ ID NO: 556 - DNA, 18 bp, Artificial Sequence
  Oligonucleotide Detection Strand
acctgcatcgaattccac

SEQ ID NO: 557 - DNA, 18 bp, Artificial Sequence
  Oligonucleotide Detection Strand
acgctcatcgaattccac

SEQ ID NO: 558 - DNA, 18 bp, Artificial Sequence
  Oligonucleotide Detection Strand
acccagatcgaattccac

SEQ ID NO: 559 - DNA, 18 bp, Artificial Sequence
  Oligonucleotide Detection Strand
acttccgtcgaattccac

SEQ ID NO: 560 - DNA, 18 bp, Artificial Sequence
  Oligonucleotide Detection Strand
accatcgtcgaattccac

SEQ ID NO: 561 - DNA, 18 bp, Artificial Sequence
  Oligonucleotide Detection Strand
accgacttcgaattccac

SEQ ID NO: 562 - DNA, 18 bp, Artificial Sequence
  Oligonucleotide Detection Strand
acgaccttcgaattccac

SEQ ID NO: 563 - DNA, 18 bp, Artificial Sequence
  Oligonucleotide Detection Strand
acccctttcgaattccac

SEQ ID NO: 564 - DNA, 18 bp, Artificial Sequence
  Oligonucleotide Detection Strand
acccaatccgaattccac

SEQ ID NO: 565 - DNA, 42 bp, Artificial Sequence
  Decoy DNA
cactacaacacatccctcaccgtcaacactccattccctcac

(19) United States
(12) Patent Application Publication
(10) Pub. No.: US 2009/0163371 A1
(43) Pub. Date: Jun. 25, 2009

(54) Anchor-Assisted Fragment Selection and Directed Assembly

(76) Inventors: Andrew M. Stern, Boston, MA (US); Scott L. Harbeson, Cambridge, MA (US); Edward M. Driggers, Arlington, MA (US); Kerry L. Spear, Concord, MA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 11/915,533

(22) PCT Filed: May 31, 2006
(86) PCT No.: PCT/US06/21088

§ 371 Date: Sep. 12, 2008

Related U.S. Application Data
(60) Provisional application No. 60/686,000, filed on May 31, 2005.
(60) Provisional application No. 60/711,497, filed on Aug. 26, 2005.
(60) Provisional application No. 60/800,496, filed on May 15, 2006.

(51) Int. Cl. C40B 30/04; C12Q 1/68

(52) U.S. Cl. 506/9; 435/6

(57) ABSTRACT

The invention provides methods for compound and lead generation and discovery. In particular, the present invention provides a method for generating compounds and for selecting compounds that bind to a target. The present invention provides a way by which anchors (e.g., weak binders) and anchor-scaffold conjugates can be evolved into new generations of compounds having improved target binding and other desired pharmaceutical properties through control of both synthetic input and selection criteria.

### RELATED APPLICATIONS

This application claims the benefit of and priority to U.S. Patent Applications Ser. Nos. 60/686,000, filed on May 31, 2005; 60/711,497, filed on Aug. 26, 2005; and 60/800,496, filed on May 15, 2006, the entire disclosure of each of which is incorporated by reference herein for all purposes.

### FIELD OF THE INVENTION

The present invention relates generally to DNA programmed chemistry and generation and discovery of compounds for target binding. More particularly, the present invention relates to methods for making and identifying organic molecules for binding to biological targets through anchor and/or fragment-based nucleic acid-templated chemistry.

### BACKGROUND

Although chemistry and screening throughputs have increased significantly recently, drug lead discovery and development remain a high-risk, low-return process. An initial task in the generation of novel, biologically effective molecules is to identify and characterize binding ligands for a given biological target molecule. To date, this continues to be a daunting task in drug lead discovery. While many millions of compounds have been synthesized and screened, few have led to optimized compounds that eventually meet all the requirements of a drug.

More recently, fragment-based approaches for compound discovery have started to emerge. Small, diverse and information-rich fragments may provide more chemical space for optimization. Moreover, fragments of low complexity may be more likely to match a target binding site. As a result, certain compounds may still provide good starting points for optimization. Examples of such approaches include the “SAR by NMR” approach developed by Fesik et al. (U.S. Pat. No. 5,698,401 by Fesik et al.; Shuker, et al., 1996, Science, vol. 274, pp. 1531-1534), the “tethering” approach pioneered by Wells, et al. (U.S. Pat. No. 6,335,155 by Wells, et al.; Erlanson, et al., 2000, PNAS, vol. 97(17), pp. 9367-9372), a high-throughput x-ray crystallography method by Carr et al. (Carr, et al., 2002, Drug Discovery Today, vol. 7, pp. 522-527), and the use of surface plasmon resonance developed by Vetter et al. (Vetter, J., 2002, Cell. Biochem. Suppl., vol. 39, pp. 79-84).

In a manner analogous to the method of pharmacophore recombination (see, U.S. Pat. No. 6,344,334 by Ellman et al.), these methods identify fragments that bind to biological targets of interest and then elaborate them into novel structures with greater affinity for the target. The structure-based methods then apply knowledge of the fragments bound to the binding site to the design of new ligands. Reactive functional groups on the fragments are utilized in pharmacophore recombinations to enable chemical assembly of the identified fragments in a combinatorial fashion to produce a library of new ligands that may have greater affinity for the target. The desired outcome of these methods is the identification of a drug lead compound that binds to a biological target of therapeutic interest.

These methods, however, suffer from several deficiencies. One is the requirement for large amounts of protein for use in the required structural studies (either X-ray or NMR). Relatively large amounts of target protein are also required for the biological screen required to test each fragment member individually for its ability to inhibit or bind the target. Because of the biological screening requirement, another issue is the requirement for fragments that are not only soluble but also well behaved under the assay conditions in the 10 μM to 1 mM (or higher) range. At these high concentrations, non-specific effects such as aggregation of the fragment molecules can yield erroneous or misleading results.

U.S. Pat. No. 6,335,155 describes a method for hit discovery that employs a covalent bond (a disulfide bond) to form a target/ligand conjugate in order to facilitate identification of organic ligands. This “tethering approach” is similarly used in U.S. Pat. No. 6,811,966 and US2002/0150947. These methods, however, suffer from several deficiencies. One is the requirement of the identification of a reactive group on the target molecule (or the introduction of a reactive group) that can be used to form a covalent bond with a ligand. Structural information of the target is therefore necessary. Another limitation is that the covalent bond between the target protein and the ligand limits screening to only a small area adjacent to the covalent bond, thereby leaving other areas of potential binding sites unexplored. Furthermore, the need for a disulfide bond limits the diversity of ligands that may be screened by these methods.

In another approach, self-assembling chemical libraries have been reported where such libraries are used for the identification of molecules for target binding. Organic molecules are linked to individual oligonucleotides that mediate the self-assembly of the library and provide a code associated with the organic molecules. See, e.g., U.S. Patent Application Publication No. 2004/0014090 A1 by Neri et al. and PCT International Publication No. WO 03/076943 A1.

While these and other approaches have provided additional tools for compound discovery, there is still a need for a more efficient and effective way of generating and selecting compounds for various pharmaceutical and other needs.

### SUMMARY OF THE INVENTION

The present invention is based, in part, upon the discovery that nucleic acid-templated chemistry can be applied to compound and drug lead discovery in a way that greatly increase the efficiency of compound and drug lead generation and discovery. In particular, the present invention provides a unique way of generating drug-like compounds and selecting compounds for target binding. The present invention further provides a way by which compounds (e.g., compounds of low complexity) and compound fragments can be evolved from initial fragments into new generations of compounds having improved target binding and other desired pharmaceutical properties through control of both synthetic input and selection criteria. The present invention further provides a way by which anchors (e.g., weak binders) and anchor-scaffold (or -fragment/building blocks) conjugates can be evolved into new generations of compounds having improved target binding and other desired pharmaceutical properties through control of both synthetic input and selection criteria.

In the methods described herein, a nucleic acid molecule functions not only as a detection strand for identification of fragments that bind to a target but also templates the chemical assembly of those fragments (e.g., in a directed combinatorial approach) to achieve combinations of fragments into ligands of enhanced affinity. Fragment selection and directed assembly by nucleic acid-templated chemistry permits the identification of pharmacophores and their subsequent assembly into novel ligands with high affinity for the target. Unlike other methods that require each fragment molecule to be assayed individually, the methods of the present invention allow selection of fragment libraries, identification of multiple fragments simultaneously, and determination of the relative affinities of the fragments, which provides structure-activity relationship (SAR) data that can be used in the design of the building blocks for use in the subsequent fragment assembly.

In one aspect, the invention provides a method for identifying a target binding element capable of binding to a binding domain disposed within a binding site of a target molecule. A target molecule is combined with a plurality of pre-selected test molecules under conditions that permit a test molecule to bind to a binding domain of the target molecule. Each test molecule includes a target binding element that is associated with a corresponding oligonucleotide. The oligonucleotide has a nucleotide sequence that (i) identifies the target binding element, (ii) contains an amplification sequence, and (iii) is substantially incapable of hybridizing to (i.e., does not hybridize to) the nucleotide sequence associated with other test molecules. A target binding element is harvested that binds to the target molecule binding site with a K_{D} of 10 mM or lower. The sequence of the oligonucleotide associated with the target binding element harvested is determined so as to identify the target binding element that binds with a K_{D} of 10 mM or lower. In one embodiment, the oligonucleotide associated with the target binding element harvested is amplified. The sequence of the amplified oligonucleotide is determined so as to identify the target binding element that binds with a K_{D} of 10 mM or lower. In this method, each of substantially all of the target binding elements has at least one of the following characteristics: (i) a c Log P between −2 and 4, (ii) 4 or fewer H-bond donors, (iii) 8 or fewer H-bond acceptors, and (iv) a molecular weight between 90 and 500 daltons.

In another aspect, the invention provides a method for identifying a target binding element capable of binding to a binding domain disposed within a binding site of a target molecule. The target binding elements so identified bind with a K_{D} of 10 mM or lower. A target molecule is combined with a plurality of pre-selected test molecules under conditions that permit a test molecule to bind to a binding domain of the target molecule. Each test molecule includes a target binding element that is associated with a corresponding oligonucleotide. The oligonucleotide has a nucleotide sequence that (i) identifies the target binding element, (ii) contains an amplification sequence, and (iii) is substantially incapable of hybridizing (i.e., or does not hybridize) to the nucleotide sequences associated with other target binding elements. A target binding element is harvested that binds to the target molecule with a K_{D} of 10 mM or lower. The oligonucleotide associated with the target binding element harvested is amplified. The sequence of the amplified oligonucleotide is determined so as to identify the target binding element having a K_{D} with the binding site of 10 mM or lower.

In yet another aspect, the invention provides an in vitro method for producing a molecule that binds to a pre-selected target molecule. The pre-selected target molecule includes a binding site that includes a first binding domain and a second binding domain. A template and a reagent are provided. The template includes a first target binding element attached to a first oligonucleotide that defines a first codon sequence. The first target binding element has a first K_{D} with the first binding domain of the binding site. The reagent includes a second target binding element attached to a second oligonucleotide that defines a first anti-codon sequence capable of hybridizing to the codon sequence. The second target binding element has a second K_{D} with the second binding domain. The template and the reagent are combined under conditions to permit the first codon sequence to hybridize to the first anti-codon sequence so as to bring the first and second target binding elements into reactive proximity. The first and second target binding elements are chemically coupled (e.g., in the absence of a ribosome) to produce a reaction product that binds to the preselected target molecule. In an embodiment, the reaction product has a K_{D} with the binding site less than (i) the first K_{D} of the first target binding element with the first binding domain, and (ii) the second K_{D} of the second target binding element with the second binding domain.

In yet another aspect, the invention provides a composition that includes a plurality of test molecules. Each of substantially all of the test molecules includes a target binding element associated with a corresponding oligonucleotide. The oligonucleotide has a nucleotide sequence that (i) identifies the target binding element, (ii) contains an amplification sequence, and (iii) is substantially incapable of hybridizing to the nucleotide sequences associated with other target binding elements.

In yet another aspect, the invention provides a composition that includes a plurality of test molecules. Each of at least some of the test molecules includes two or more target binding elements and is associated with a corresponding oligonucleotide. The oligonucleotide has a nucleotide sequence that (i) identifies the two or more target binding elements, (ii) contains an amplification sequence, and (iii) is substantially incapable of hybridizing to the nucleotide sequences associated with other test molecules.

In yet another aspect, the invention provides a composition that includes a plurality of test molecules. Each of substantially all of the test molecules comprises two or more target binding elements and is associated with a corresponding oligonucleotide. The nucleotide has a nucleotide sequence that (i) identifies the two or more target binding elements, (ii) contains an amplification sequence, and (iii) is substantially incapable of hybridizing to the nucleotide sequences associated with other test molecules.

In yet another aspect, the invention provides a complex of a target molecule bound to a test molecule. The test molecule includes two or more target binding elements. The test molecule is associated with a corresponding oligonucleotide that has a nucleotide sequence that (i) identifies the test molecule and (ii) contains an amplification sequence. Each of substantially all of the target binding elements has at least one of the following characteristics: (i) a c Log P between −2 and 4, (ii) 4 or fewer H-bond donors, (iii) 8 or fewer H-bond acceptors, and (iv) a molecular weight between 90 and 500 daltons.

In yet another aspect, the invention provides a composition that includes a plurality of complexes. Each complex includes a target molecule bound to a test molecule. The test molecule includes two or more target binding elements. Each test molecule is associated with a corresponding oligonucleotide. The oligonucleotide has a nucleotide sequence that (i) identifies the test molecule, (ii) contains an amplification sequence, and (iii) is substantially incapable of hybridizing to the nucleotide sequence associated with other test molecules. Each of substantially all of the target binding elements is linked to a functional group through which the target binding element is attached to the oligonucleotide.

In yet another aspect, the invention provides a composition that includes a plurality of complexes. Each complex includes a target molecule bound to a test molecule that includes two or more target binding elements. Each test molecule is associated with a corresponding oligonucleotide that has a nucleotide sequence that (i) identifies the test molecule, (ii) contains an amplification sequence, and (iii) is substantially incapable of hybridizing to the nucleotide sequences of other test molecules.

In yet another aspect, the invention provides a method for identifying a target binding element capable of binding to a binding domain disposed within a binding site of a target molecule. The target binding elements so identified bind with a K_{d} of 10 mM or lower. A target molecule is combined with a plurality of pre-selected test molecules under conditions that permit a test molecule to bind to a binding domain of the target molecule. Each test molecule includes a target binding element that is associated with a corresponding oligonucleotide. The oligonucleotide has a nucleotide sequence that (i) identifies the target binding element, (ii) contains an amplification sequence, and (iii) is substantially incapable of hybridizing (i.e., or does not hybridize) to the nucleotide sequences associated with other target binding elements. A target binding element is harvested that binds to the target molecule with a K_{d} of 10 mM or lower. The oligonucleotide associated with the target binding element harvested is amplified. The sequence of the amplified oligonucleotide is determined so as to identify the target binding element having a K_{d} with the binding site of 10 mM or lower.

In yet another aspect, the invention provides an in vitro method for producing a molecule that binds to a pre-selected target molecule. The pre-selected target molecule includes a binding site that includes a first binding domain and a second binding domain. A template and a reagent are provided. The template includes a first target binding element attached to a first oligonucleotide that defines a first codon sequence. The first target binding element has a first K_{d} with the first binding domain of the binding site. The reagent includes a second target binding element attached to a second oligonucleotide that defines a first anti-codon sequence capable of hybridizing to the codon sequence. The second target binding element has a second K_{d} with the second binding domain. The template and the reagent are combined under conditions to permit the first codon sequence to hybridize to the first anti-codon sequence so as no to bring the first and second target binding elements into reactive proximity. The first and second target binding elements are chemically coupled (e.g., in the absence of a ribosome) to produce a reaction product that has a K_{d} with the binding site less than (i) the first K_{d} of the first target binding element with the first binding domain, and (ii) the second K_{d} of the second target binding element with the second binding domain.

In yet another aspect, the invention provides a method for identifying a target binding element capable of binding to a binding domain disposed within a binding site of a target molecule. A target molecule is combined with a plurality of test molecules under conditions that permit a test molecule to bind to a binding domain of the target molecule. Each test molecule includes a target binding element that is associated with a corresponding oligonucleotide. The oligonucleotide has a nucleotide sequence that (i) identifies the target binding element, (ii) contains an amplification sequence, and (iii) is substantially incapable of hybridizing to (i.e., does not hybridize to) the nucleotide sequence associated with other test molecules. A target binding element is harvested that binds to the target molecule binding site with a K_{d} of 10 mM or lower. The sequence of the oligonucleotide associated with the target binding element harvested is determined so as to identify the target binding element that binds with a K_{d} of 10 mM or lower. In one embodiment, the oligonucleotide associated with the target binding element harvested is amplified. The sequence of the amplified oligonucleotide is determined so as to identify the target binding element that binds with a K_{d} of 10 mM or lower. In this method, each of substantially all of the target binding elements has at least one of the following characteristics: (i) a c Log P between −2 and 4, (ii) 4 or fewer H-bond donors, (iii) 8 or fewer H-bond acceptors, and (iv) a molecular weight between 90 and 500 daltons.

In yet another aspect, the invention provides a method for identifying a target binding element capable of binding to a target molecule. A target molecule is combined with a plurality of test molecules under conditions that permit a test molecule to bind to a binding domain of the target molecule. Each test molecule includes a target binding element that is associated with a corresponding oligonucleotide. The oligonucleotide has a nucleotide sequence that (i) identifies the target binding element, (ii) contains an amplification sequence, and (iii) is substantially incapable of hybridizing to (i.e., does not hybridize to) the nucleotide sequence associated with other test molecules. A target binding element is harvested that binds to the target molecule binding site with a K_{d} of 10 mM or lower. The sequence of the oligonucleotide associated with the target binding element harvested is determined so as to identify the target binding element that binds with a K_{d} of 10 mM or lower. In one embodiment, the oligonucleotide associated with the target binding element harvested is amplified. The sequence of the amplified oligonucleotide is determined so as to identify the target binding element that binds with a K_{d} of 10 mM or lower. In this method, each of substantially all of the target binding elements has all of the following characteristics: (i) a c Log P between −2 and 4, (ii) 4 or fewer H-bond donors, (iii) 8 or fewer H-bond acceptors, and (iv) a molecular weight between 90 and 500 daltons.

In yet another aspect, the invention provides a method for identifying a compound having a desired binding affinity to a target molecule. The method includes the following. A library is provided that includes a plurality of test compounds. Each of the test compounds includes (1) a common binding moiety, (2) a scaffold moiety connected to the common binding moiety through a bridging moiety, and (3) an oligonucleotide having a nucleotide sequence informative of the structural or synthetic information of the associated test compound. The common binding moiety has a dissociation constant of 10 mM or lower to a first binding domain of the target molecule. A reference compound is provided that includes the common binding moiety. The target molecule, the library of test compounds, and the reference compound are combined under conditions that permit the plurality of test compounds and the reference compound to compete for binding to the target molecule. The test compounds that exhibit greater binding affinity to the target molecule than the reference compound are harvested. The oligonucleotide sequences of the test compounds harvested are determined thereby to identify the test compounds having a desired binding affinity to the target molecule.

In yet another aspect, the invention provides a method for identifying a compound having a desired binding affinity to a target molecule. The method includes the following. The target molecule, a plurality of test compounds, and a reference compound are combined under conditions that permit the plurality of test compounds and the reference compound to compete for binding to the target molecule. Each of the plurality of test compounds includes (1) a common binding moiety, (2) a scaffold moiety connected to the common binding moiety through a bridging moiety, and (3) an oligonucleotide having a nucleotide sequence informative of the structure or synthetic information of the associated test compound. The reference compound includes the common binding moiety. The common binding moiety has a dissociation constant of 10 mM or lower to a first binding domain of the target molecule. The oligonucleotide sequences of the test compounds that bound to the target are determined.

In yet another aspect, the invention provides a method for detecting a second binding domain on a target molecule having a first binding domain. The method includes the following. A test compound is provided that includes (1) a first binding moiety having a binding affinity to the first binding domain of the target molecule, (2) a scaffold moiety connected to the first binding moiety through a bridging moiety, and (3) a defining oligonucleotide having a nucleotide sequence informative of the structure or synthetic information of the test compound. The first binding moiety has a dissociation constant of 10 mM or lower to a first binding domain of the target molecule. The effect of the test compound on the binding of a reference compound to the target molecule is determined. The reference compound comprises the first binding moiety. The data collected is analyzed to detect the presence of a second binding domain on the target molecule.

In yet another aspect, the invention provides a method for identifying a compound having a desired binding affinity to a target molecule. The method provides the following. A library is provided that includes a plurality of test compounds, wherein each of the test compound comprises (1) a common binding moiety, (2) a scaffold moiety connected to the common binding moiety through a bridging moiety, and (3) an oligonucleotide having a nucleotide sequence informative of the structural or synthetic information of the associated test compound. The common binding moiety has a dissociation constant of 10 mM or lower to a first binding domain of the target molecule. The target molecule and the plurality of test compound are combined under conditions that permit binding of one or more of the plurality of test compounds to the target molecule if such test compounds with desired binding affinity are present. The test compounds bound to the target are harvested. The oligonucleotide sequences of the test compounds harvested are determined thereby identifying the test compounds having a desired binding affinity to the target molecule.

In yet another aspect, the invention provides a method for selecting a compound having a desired binding affinity to a target molecule. The method includes the following. A library is provided that includes two subsets of test compounds. Each of the first subset of test compounds includes (1) a common binding moiety, (2) a first scaffold moiety connected to the common binding moiety through a bridging moiety, and (3) an oligonucleotide having a nucleotide sequence informative of the structural or synthetic information of the associated test compound. The common binding moiety has a dissociation constant of 10 mM or lower to a first binding domain of the target molecule. Each of the second subset of test compounds includes (1) a second scaffold moiety, and (2) an oligonucleotide having a nucleotide sequence informative of the structural or synthetic information of the associated test compound. The first scaffold and the second scaffold may be the same scaffold. A reference compound is provided that includes the common binding moiety. The target molecule, the library of test compounds, and the reference compound are combined under conditions that permit the plurality of test compounds and the reference compound to compete for binding to the target molecule. The test compounds that exhibit greater binding affinity to the target molecule than the reference compound are harvested. The oligonucleotide sequences of the test compounds harvested are determined thereby to identify the test compounds having a desired binding affinity to the target molecule.

In yet another aspect, the invention provides a library of chemical compounds. The library includes a plurality of compounds. The compounds are prepared by one or more nucleic acid-templated chemical reactions. Each of the compounds comprises (1) a first moiety, (2) a second moiety connected to the first moiety through a bridging moiety, and (3) an oligonucleotide having a nucleotide sequence informative of the structure or synthetic information of the second moiety. The first moiety has a dissociation constant of 10 mM or lower less to a binding domain of the target molecule.

In yet another aspect, the invention provides a compound. The compound comprises (1) a first moiety, (2) a second moiety connected to the first moiety through a bridging moiety, e and (3) an oligonucleotide having a nucleotide sequence informative of the structure or synthetic information of the second moiety. The first moiety has a dissociation constant of 10 mM or lower less to a binding domain of the target molecule.

The foregoing aspects and embodiments of the invention may be more fully understood by reference to the following definitions, figures, detailed description and claims.

### DEFINITIONS

The term, “anchor” as used herein, refers to a small molecule fragment, a small molecule or peptide having preselected binding affinity for a target, preferably (but not necessarily) with a molecular weight less than 250 daltons. An anchor may or may not contain further functionalization to facilitate subsequent DNA programmed chemistry.

The term, “amplification” or to “amplify”, as used herein, relates to the production of additional copies of a nucleic acid sequence. Amplification is generally carried out using polymerase chain reaction (PCR) technologies well known in the art. (See, e.g., Dieffenbach, et al., 1995, PCR Primer, a Laboratory Manual, Cold Spring Harbor Press, Plainview, N.Y., pp. 1-5.).

It is contemplated, however, that amplification may not be necessary to conduct the methods of the present invention where the oligonucleotide sequences of interest (e.g., those that identify target binding elements and members of chemical libraries synthesized by DNA programmed chemistry) may be determined by methods that do not require amplification of the sequences (e.g., direct sequencing). Thus, where herein amplification is described as a step or a process, sequencing without prior amplification of an oligonucleotide is also contemplated.

The term, “associated with” as used herein describes the interaction between or among two or more groups, moieties, compounds, monomers, etc. When two or more entities are “associated with” one another as described herein, they are linked by a direct or indirect covalent or non-covalent interaction. Preferably, the association is covalent. The covalent association may be, for example, but without limitation, through an amide, ester, carbon-carbon, disulfide, carbamate, ether, thioether, urea, amine, or carbonate linkage. The covalent association may also include a linker moiety, for example, a photocleavable linker. Desirable non-covalent interactions include hydrogen bonding, van der Waals interactions, dipole-dipole interactions, pi stacking interactions, hydrophobic interactions, magnetic interactions, electrostatic interactions, etc.

The term, “bind” or “binding” as used herein in connection with the interaction between a target (e.g., a protein) and a potential binding compound indicates that the potential binding compound associates with the target to a statistically significant degree as compared to association with similar targets (e.g., proteins) generally (i.e., non-specific binding). Thus, a compound binds to a target when the compound has a statistically significant association with a target molecule. Preferably a binding compound interacts with a specified target with a dissociation constant (K_{D} or K_{d}) of 10 mM or less. A binding compound can bind with “extremely low affinity” (1 mM<K_{D}<10 mM), “very low affinity” (100 μM<K_{D}<1 mM), “low affinity” (10 μM<K_{D}<100 μM), “moderate affinity” (1 μM<K_{D}<10 μM), “moderately high affinity” (100 nM<K_{D}<1 μM), or “high affinity” (K_{D}<100 nM, e.g., K_{D}<50 nM or 20 nM, or “very high affinity” (1 nM or sub-nanomolar<K_{D}<10 nM)) depending on the dissociation constant.

The term, “binding site” as used herein, refers to an area on a target molecule that participate in molecular recognition by a binding compound. Binding sites embody particular shapes and often contain multiple binding domains (or “binding pockets”) present within the binding site and collectively represent the binding site. By “binding domain” or “binding pocket” is meant a specific volume within a binding site. A binding domain can often be a particular shape, indentation or cavity in the binding site. Binding domains can contain particular chemical groups or structures that are important in the non-covalent binding of another molecule such as, for example, groups that contribute to ionic, hydrogen bonding, or van der Waals interactions between the molecules. The binding site or domains may be known in advance, or discovered in the process of implementing the procedures described herein.

The term, “codon” and “anti-codon” as used herein, refer to complementary oligonucleotide sequences in a template strand and in a reagent (or transfer) strand, respectively, that permit the reagent strand to anneal to the template strand during DNA programmed chemistry. Codons on templates identify or encode the small molecules attached to the templates according to the reagents and/or target binding elements used and the chemical transformation performed. Anti-codons on reagent strands or a solid support interact through Watson-Crick base pairing with codons (i.e., specific sub-sequences within templates) in DNA programmed chemistry, thereby specifically delivering selected reagents (including, e.g., target binding elements) to the template in the DNA programmed chemistry process.

The term, “common binding moiety” as used herein, refers to an anchor moiety that is incorporated into an expanded molecule comprising the anchor moiety and a scaffold, fragment or building blocks.

The terms “complementary” as used herein, refer to the natural binding of polynucleotides under permissive salt and temperature conditions by base pairing. For example, the sequence “A-G-T” binds to the complementary sequence “T-C-A.” Complementarity between two single-stranded molecules may be “partial,” such that only some of the nucleic acids bind, or it may be “complete,” such that total complementarity exists between the single stranded molecules. The degree of complementarily between nucleic acid strands has significant effects on the efficiency and strength of the hybridization between the nucleic acid strands.

The term, “detection strand” as used herein, refers to an oligonucleotide that includes a specific identification sequence and may include PCR primer binding sequences. The specific identification sequence identifies the fragment or molecule associated with the detection strand, and can be covalently attached via linker to a target binding elements. The specific identification sequence additionally is designed to ensure an absence of base-pairing with other detection strands.

The term, “K_{D}” or “apparent K_{d}” as used herein, refers to apparent dissociation constant as defined below.

K_{d}(or dissociation constant)={[P]·[L]}/[P·L]

where P is the target (e.g., protein) and L is a specific library member with the potential to bind to P.

K_{D}(or apparent dissociation constant)={[P]_{T}·(1−ε·N_{SB})}/ε·N_{SB}

where ε (or observed enrichment of L relative to all library members)={[P·L]/[L]_{T}}/N_{SB};

N_{SB} is the non-specific background of total library bound in the absence of P expressed as a fraction of total library; [P]_{T} represents total target concentration; [L]_{T} represents the total specific ligand concentration. For [P]_{T}>>[L]_{T}, [P]=[P]_{T}, [L]_{T}=[P·L]+[L].

The term, “DNA programmed chemistry” (or “DPC”) or “nucleic acid-templated chemistry” as used herein, refer to a method by which synthetic products are translatable into amplifiable information via oligonucleotide templates. Particularly, sequence specific control of chemical reactants to yield specific products is accomplished by (1) providing one or more templates, which have associated reactive units; (2) contacting one or more transfer units (reagents) having an anti-codon and reactive unit with one or more templates under conditions to allow for hybridization to the templates and (3) reaction of the reactive units to yield products (e.g., products being associated with an amplifiable template). The structures of the reactants and products need not be related to those of the nucleic acids of the template and transfer unit.

The term, “DPC-fragment” as used herein, refers to the molecular combination of a target binding element covalently linked to a nucleotide strand (e.g., via a linker) in such a way that the molecular combination can participate directly in a DPC process (and optionally also is functionalized for subsequent DPC processes). The nucleotide strand is a detection strand or a reagent strand that includes an anti-codon (selected to enable binding to a DPC template) and PCR primer binding sequences to enable amplification of the sequence.

The term, “hybridization” as used-herein, refers to any process by which a strand of nucleic acid binds with a complementary strand through base pairing.

The term, “linker” as used herein, refers to any of a number of molecular entities (cleavable or non-cleavable) that can be used to covalently attach functionalized small molecules to their respective DPC reagent, template or detection strands.

The terms, “nucleic acid”, “oligonucleotide” or “oligo” or “polynucleotide” as used herein refer to a polymer of nucleotides. The polymer may include, without limitation, natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages). Nucleic acids and oligonucleotides may also include other polymers of bases having a modified backbone, such as a locked nucleic acid (LNA), a peptide nucleic acid (PNA), a threose nucleic acid (TNA) and any other polymers capable of serving as a template for an amplification reaction using, an amplification technique, for example, a polymerase chain reaction, a ligase chain reaction, or non-enzymatic template-directed replication.

The term “plurality” or “set” as used in a “plurality” or “set” of fragments or compounds is meant a collection of fragments or compounds. The fragments or compounds may or may not be structurally related. For example, the number of fragments or compounds may be anywhere from 10; 20; 50; 100; 1,000; 10,000; 100,000; 500,000; to 1,000,000 or more.

The term, “reagent strand” as used herein, refers to an oligonucleotide that include an anti-codon (and may include but does not require PCR primer sequences) that are associated with (e.g., covalently) a small molecule, which may be a target binding element, or any other molecular species that can participate in a DPC process.

The term, “reference compound” as used herein, refers to a compound that comprises the common binding moiety that retains the binding characteristics of the common binding moiety.

The term, “scaffold” as used herein, refers to a chemical compound having at least one site or chemical moiety suitable for functionalization. For example, a small molecule scaffold or molecular scaffold may have two, three, four, five or more sites or chemical moieties suitable for functionalization. These functionalization sites may be protected or masked as would be appreciated by a person of ordinary skill in the art. The sites may also be found on an underlying ring structure or backbone.

The term, “small molecule” as used herein, refers to an organic compound either synthesized in the laboratory or found in nature having a molecular weight less than 10,000 daltons, optionally less than 5,000 daltons, and optionally less than 1,500 daltons. Preferably, a small molecule has a molecular weight less than 1,000 daltons, optionally less than 500 daltons, and optionally less than 250 daltons.

The term, “target” as used herein, refers to any compound of interest, small molecule or polymeric, naturally occurring or non-naturally occurring, and biological molecules or otherwise. A target can be an enzyme, protein, peptide, carbohydrate, polysaccharide, glycoprotein, hormone, receptor, antigen, antibody, virus, substrate, metabolite, transition state analog, cofactor, inhibitor, drug, dye, nutrient, growth factor, cell, tissue etc., without limitation. For example, the binding region of a target molecule may include a catalytic site of an enzyme, a binding pocket on a receptor (e.g., a G-protein coupled receptor), a protein surface area involved in a protein-protein or protein-nucleic acid interaction (e.g., a hot-spot region), or a specific site on DNA (e.g., the major groove) or a site with no biological function. The natural function of the target could be stimulated (agonized), reduced (antagonized), unaffected, or completely changed by the binding depending on the precise binding mode and the particular binding site. A target can also be a surface of a material, e.g., the surface or coating of a polymeric material or a metallic material.

For example, a target and a small molecule having binding affinity toward the target may form a non-covalently interaction to associate the target with the binding molecule. Non-covalent binding includes the subsequent introduction of functional groups into the small molecule compound that causes covalent attachment to the target following the non-covalent molecular recognition and binding event.

Examples of targets include kinases, phosphatases, proteases, receptors, ion channels oxidases and reductases, catabolic and anabolic enzymes, pumps, and electron transport proteins.

The term, “target binding element” or “TBE” as used herein, refers to a molecule, e.g., a small molecule or peptide, a fragment, portion, framework or component thereof, that may participate in recognition and binding, for example, specific binding, to a particular target. The target binding element may bind to a binding domain of the binding domain of a target molecule.

For example, target binding elements may include small molecules or peptides with a molecular weight less than 250 daltons that may or may not have detectable affinity for a target (i.e. < or =100 μM) using non-PCR based detection methods.

The target binding elements used typically represent fragments, structures, and/or frameworks found in known drugs or leads. Additionally, these target binding elements may be linked to functional groups that enable linkage to an oligonucleotide template. These target binding elements may be linked to additional functional groups to enable their subsequent use in DPC to build libraries of more elaborated molecules.

Examples of functionalization on target binding elements include glycine as a bi-functionalized methylene fragment for DPC; methylamine or acetic acid as analogous mono-functionalized fragments for DPC; para-aminobenzoic acid as a bi-functionalized benzene fragment for DPC; aniline or benzoic acid as analogous mono-functionalized fragments for DPC; glutamine as a bifunctionalized propionamide, etc.

Target binding elements may have various affinities toward a particular target. Target binding elements may bind to the target molecule with a K_{D} or K_{d}, e.g., less than 1 nM, 10 nM, 100 nM, 1 μM, 10 μM, 20 μM, 50 μM, 100 μM, 200 μM, 500 μM, 1 mM, 100 mM, 500 mM or 1 M or greater.

The term, “template” or “DPC template” as used herein, refers to a molecule including an oligonucleotide having at least one codon sequence suitable for DNA programmed chemistry (a template mediated chemical synthesis). The template optionally may include (i) a plurality of codon sequences, (ii) an amplification means, for example, a PCR primer binding site or a sequence complementary thereto, (iii) a reactive unit associated therewith, (iv) a combination of (i) and (ii), (v) a combination of (i) and (iii), (vi) a combination of (ii) and (iii), or a combination of (i), (ii) and (iii).

For example, a template may refer to an oligonucleotide that encodes the DNA programmed synthesis of a compound that contain elaborated target binding elements to be tested for target affinity. In this case, the template includes one or more codons that recruit reagents in the DPC process, as well as PCR primer regions, and may include specific endonuclease cleavage sites.

The term, “transfer unit” as used herein, refers to a molecule including an oligonucleotide having an anti-codon sequence associated with a reactive unit including, for example, a building block, monomer, monomer unit, molecular scaffold, or other reactant useful in DNA programmed chemistry (a template mediated chemical synthesis).

Throughout the description, where compositions are described as having, including, or comprising specific components, or where processes are described as having, including, or comprising specific process steps, it is contemplated that compositions of the present invention also consist essentially of, or consist of, the recited components, and that the processes of the present invention also consist essentially of, or consist of, the recited processing steps. Further, it should be understood that the order of steps or order for performing certain actions are immaterial so long as the invention remains operable. Moreover, unless specified to the contrary, two or more steps or actions may be conducted simultaneously.

### BRIEF DESCRIPTION OF THE FIGURES

The invention may be further understood from the following figures in which:

FIG. 1 is a schematic representation of a target, binding site and binding domains in a binding site.

FIG. 2 is a schematic representation of target binding elements and corresponding DPC-fragments.

FIG. 3 is a schematic representation of an exemplary method for the discovery of target binding elements having binding affinities to a target.

FIG. 4 is a schematic representation of an exemplary method for assembly and selection of target binding elements for a target and modular iteration to refine target binding.

FIG. 5 is a schematic representation of an exemplary method for identification and selection of enriched and depleted target binding elements.

FIG. 6 is a schematic representation of one embodiment of an anchor-based approach for the identification of improved binding and novel binding sites and generation of compounds having binding affinities to such binding sites.

FIG. 7 is an exemplary set of oligonucleotide sequences useful for performing certain aspects of the present invention (presented on separate sheets).

FIG. 8 is a schematic representation of one embodiment of an anchor-based approach for the identification of improved binding and novel binding sites and generation of compounds having binding affinities to such binding sites.

FIG. 9 is a schematic representation of one embodiment of an anchor-based approach for the identification of drug hits and leads and novel binding sites.

FIG. 10 is a schematic representation of anchor conjugates.

FIG. 11 is a schematic representation of two exemplary architects of anchor conjugates.

FIG. 12 shows an example of an anchor conjugate involving macrocyclic fumaramides.

FIG. 13 is a schematic representation of an exemplary architect of a 3′ DNA conjugate.

FIG. 14 is a schematic representation of an exemplary architect of a 5′ DNA conjugate.

FIG. 15 lists exemplary target binding elements.

FIG. 16 is a schematic representation of an exemplary architect of DNA-fragment conjugated.

FIG. 17 is a schematic representation of a mix-and-split strategy for oligonucleotides and DPC fragments.

FIG. 18 shows an exemplary FOPP-labeled DPC fragment conjugate (and an anchor-fragment linked DNA conjugate).

FIG. 19 shows exemplary selections of anchor-based libraries against a biological target.

### DETAILED DESCRIPTION OF THE INVENTION

The present invention provides a new approach to drug lead generation and selection where DNA programmed chemistry plays a critical role. Key attributes of DNA programmed chemistry that make such an approach possible and effective include: 1) the extreme sensitivity of PCR-linked binding assays to identify low affinity target binding elements, 2) the ability to test directly for binding in a manner that enables discovery of novel binding modes in novel fragment combinations, 3) the ability of DPC to rapidly assemble DPC-fragments into libraries of potentially high-affinity ligands, and 4) the modularity of the DPC system to allow rapid analysis and deconvolution of binding data from an entire library of compounds synthesized from DPC fragments.

The sensitivity of a PCR-based binding assay allows detection of low affinity interactions. Interactions in the range of 10 μM to 1 mM are difficult to detect by standard biochemical screening methods in which [Ligand]>>[Target]. Without wishing to be bound by theory, this may be due to the poor aqueous solubility of many small molecules and the tendency of some of these molecules to form aggregates in solution resulting in false positives. However, these affinity ranges may represent preferred starting points for hit to lead optimization. The PCR-based binding assays can detect the presence of as few as 1 DNA molecule and provide a basis for discovering target binding elements as DPC-fragments having affinities well within this affinity range. The use of target concentrations that exceed ligand concentrations is a central component of methods designed to detect low affinity binders—an inversion of the usual concentration requirements in an in vitro binding assay.

PCR-based binding assays may allow a method of detection that is independent of any specific target and independent of any target's biochemical activity. Selections of DPC fragments or compounds therefore employ a universal binding assay. The ability to screen exclusively for binding eliminates the requisite linkage to a functional biochemical assay; therefore, binding interactions can be detected that might otherwise fail to generate the functional biochemical readout. Selections can also be performed in the presence of soluble ligands for which the binding site of the ligand to the target is known. Under these conditions of increased stringency, knowledge regarding the binding of target binding elements to the target can be inferred. This approach uniquely enables the discovery of binding sites that lie outside the scope of interactions that provide a detectable biochemical output in vitro.

DPC enables the rapid assembly of DPC-fragments into potentially high-affinity compounds. DPC-fragments can be synthesized into Compounds that may have high affinity to targets. In this novel fragment-based discovery approach, DPC-fragments identified can be assembled in a combinatorial fashion to yield libraries of more elaborated structures with an increased probability of providing moderate to high binding affinities (<<10 μM). Other fragment-based approaches have no such facile method for converting identified fragments with low affinity into larger molecular weight compounds with high target affinity. In addition, the modular nature of DPC enables assembly of a variety of scaffolds and unstructured element display methods with equivalent synthetic ease, resulting in a variety of display options for the discovered target binding elements.

A fourth key advantage is the rapid analysis and deconvolution added by the modular nature of the data that comes from the target binding deconvolution process. The modularity of the DPC-fragment based system allows fast and efficient analysis and deconvolution of binding data from an entire library of compounds synthesized from DPC fragments. The sequence analysis of the identifying oligonucleotide sequence of a target binding fragment or molecule enables the rapid identification of its structure. When such data is acquired on a whole population of compounds (e.g., target binding fragments), the relative abundance of codons that are enriched (or depleted) among the binders can be compared to their relative abundance in the original library. The availability of such data that discretely links specific codons in the DPC-fragments with the affinity contribution of specific target binding elements in those compounds (and not just the overall compound affinity), on a library-wide scale, is a unique feature of a DPC-fragment approach. This data also facilitates iteration of the discovery cycle, with the possibility of re-using modular DPC reagents in subsequent cycles of syntheses, selections and analyses.

By employing the various components of DPC on the chosen fragments, from library synthesis to binding analysis, selection and evolution of the libraries, an efficient, unique, and superior method is hereby created for compound and drug lead discovery. The present invention permits the identification of pharmacophores and their subsequent assembly into novel ligands with high affinity for the target. For example, the fragment-based approach described herein allows identification of low molecular weight binders to target proteins that serve as viable starting points for lead optimization. In addition, the present invention may be used in conjunction or combination with other methods of compound lead generation and discovery.

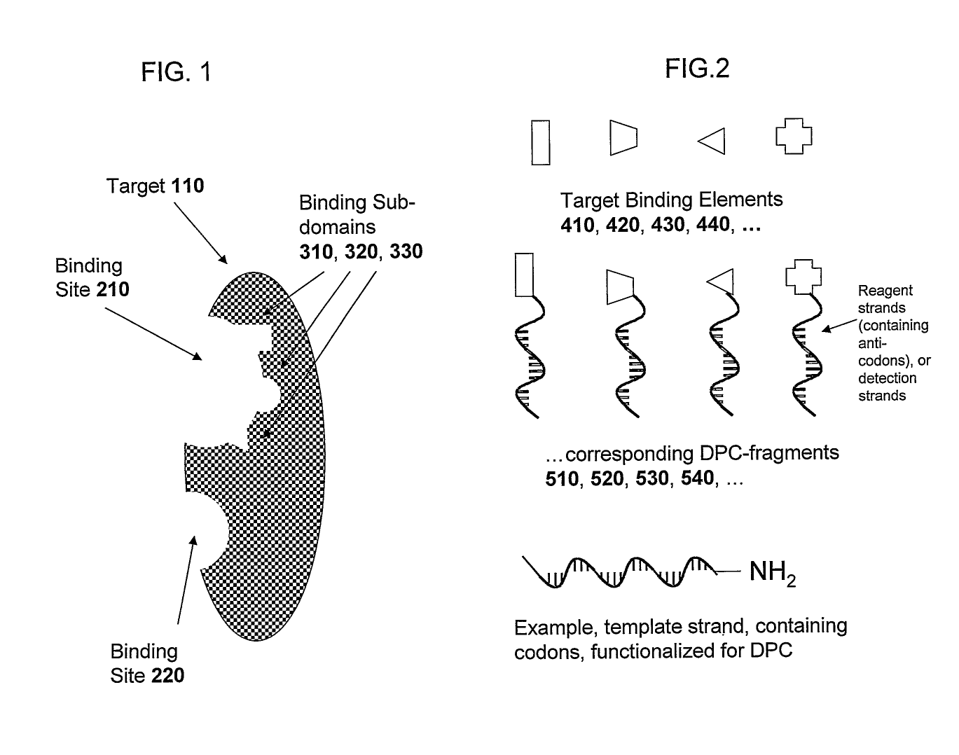

FIG. 1 schematically illustrates a target 110, one or more binding sites 210 and 220, and binding domains in a binding site 310, 320 and 330.

There is generally no limitation as to the targets that may be investigated using the methods, compositions of matters and systems of the present invention. A target can be any compound of interest, small molecule or polymeric, and biological or otherwise. The target can be an enzyme, protein, peptide, carbohydrate, polysaccharide, glycoprotein, hormone, receptor, antigen, antibody, virus, substrate, metabolite, transition state analog, cofactor, inhibitor, drug, dye, nutrient, growth factor, cell, tissue etc., without limitation. Additional examples of biological targets include kinases, phosphatases, proteases, receptors, ion channels, oxidases and reductases, catabolic and anabolic enzymes, pumps, and electron transport proteins.

FIG. 2 schematically illustrates target binding elements 410, 420, 430 and 440 and corresponding DPC-fragments 510, 520, 530 and 540. The DPC-fragments may contain a detection strand and/or a reagent strand.

Detection strands are designed to contain a primer binding sequence (for example, a 5′ PCR primer binding sequence, a 3′ PCR primer binding sequence, or both), and a specificity domain (e.g., a 4, 5, 6, 7, 8, or 10 base specificity domain). For sensitivity, the primer binding sites each include anywhere from 10 to 20 bases of sequence.

Criteria for designing the PCR primer binding sites include: 1) creating sufficient GC-content to allow annealing at an acceptable temperature, 2) minimizing palindromic sequences with respect to each other and within each primer binding site to avoid hairpin structures in the detection strand, and 3) minimization of reverse complementarity with any of the specificity domains.

Detection strands are introduced into a fragment-based discovery strategy by covalently attaching each of the strands to a pre-assigned TBE, through any of a variety of standard methods as described herein.

Detection strand sequences (including specificity domains) are designed according to the following exemplary scheme (for example using 6-mers, but can be anywhere from 4 to 20-mers): (1) a list of all possible 6-mers is constrained to the set of sequences which have GC-content >1 and <5 (20%-80%, e,g., 20%, 25%; 30%, 35%, 40%, 45%, 50%, 55%, 60%, 65%, 70%, 75%, 80%), resulting in a set of 3200 sequences; (2) these sequences are included in exemplary detection strands; (3) an edge-node graph is generated with the resulting detection strands, where every sequence (node) is connected by an edge to every other sequence; (4) connections are eliminated where, for a given pair of nodes, there is a subsequence of length S or more in common between a node and the reverse complement of the other node. S may take on a variety of values, for example 3 or 4 or 5; (5) the resulting graph is analyzed for its “maximum cliques,” which are the largest identifiable sets of nodes which are all completely inter-connected within the graph of 3200 nodes; (6) the resulting set of nodes in the maximum clique (for n=6 base codons, and S=4, a set of 510 such nodes can be found) represent detection strand sequences that are unlikely to form stable base-pairing structures between one another, and this expectation is confirmed using a standard oligo modeling program (e.g., OMP, produced by DNA Software Inc.).

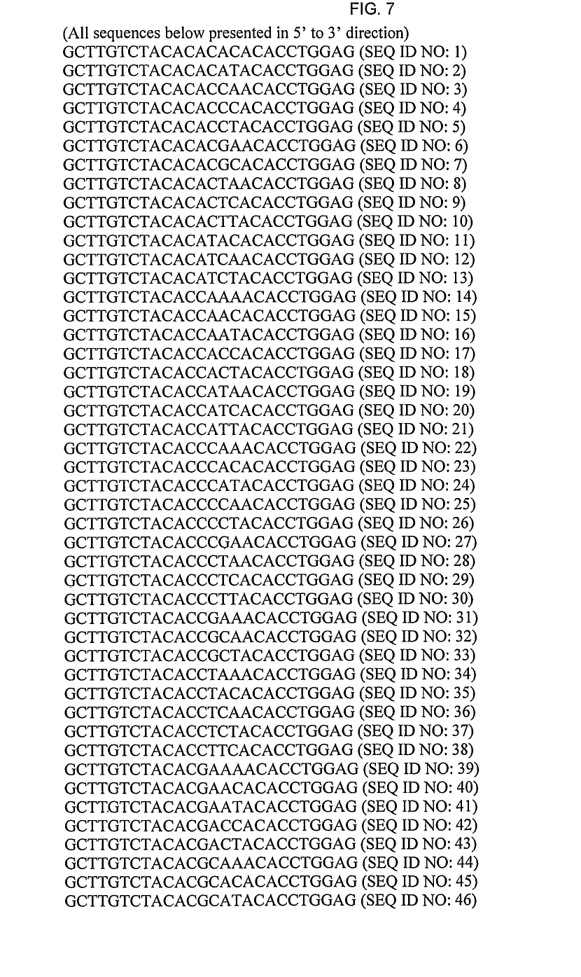

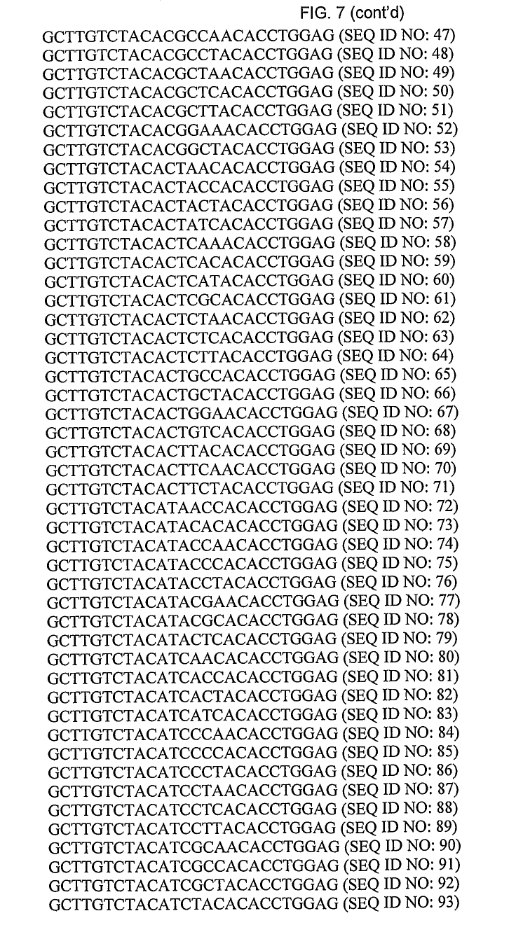

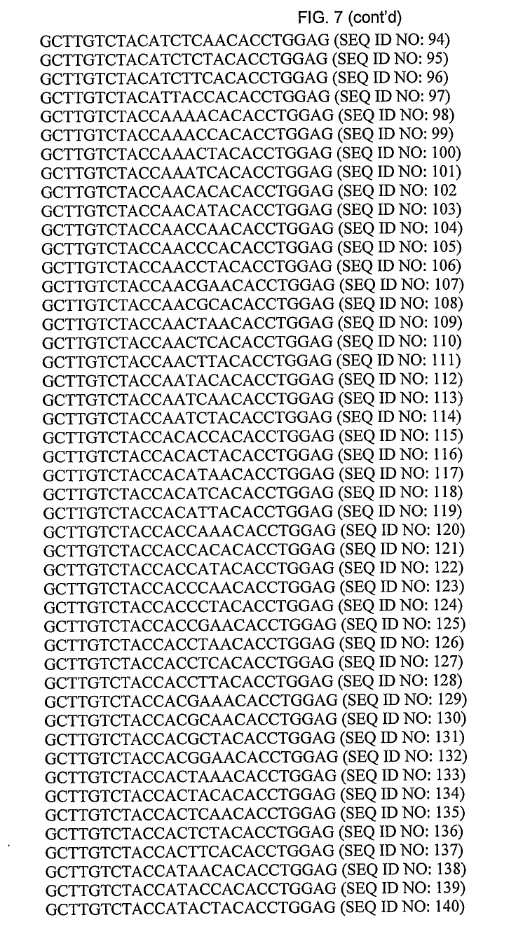

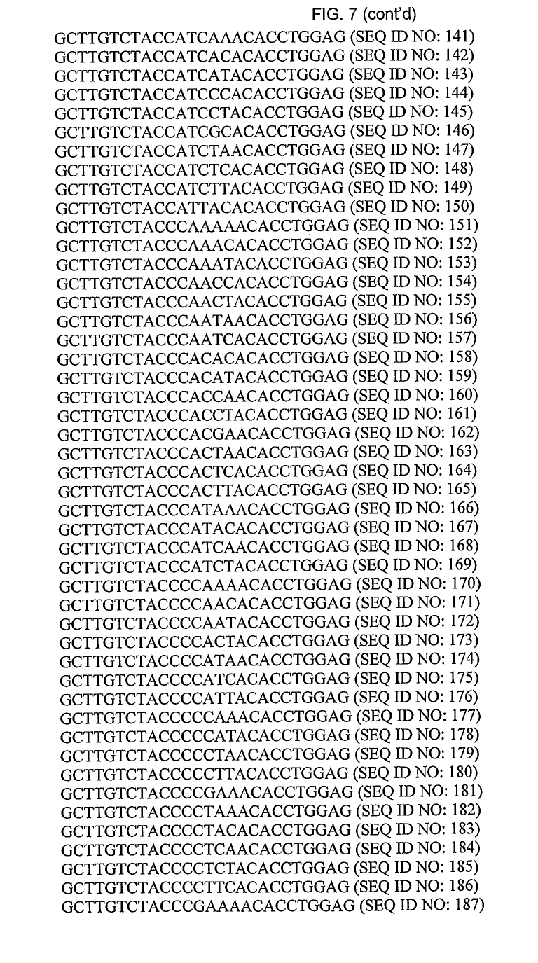

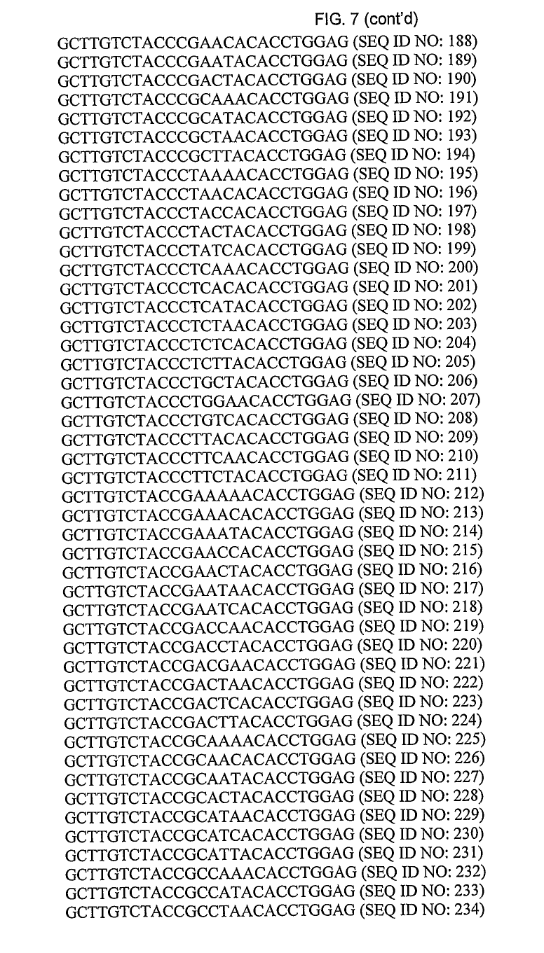

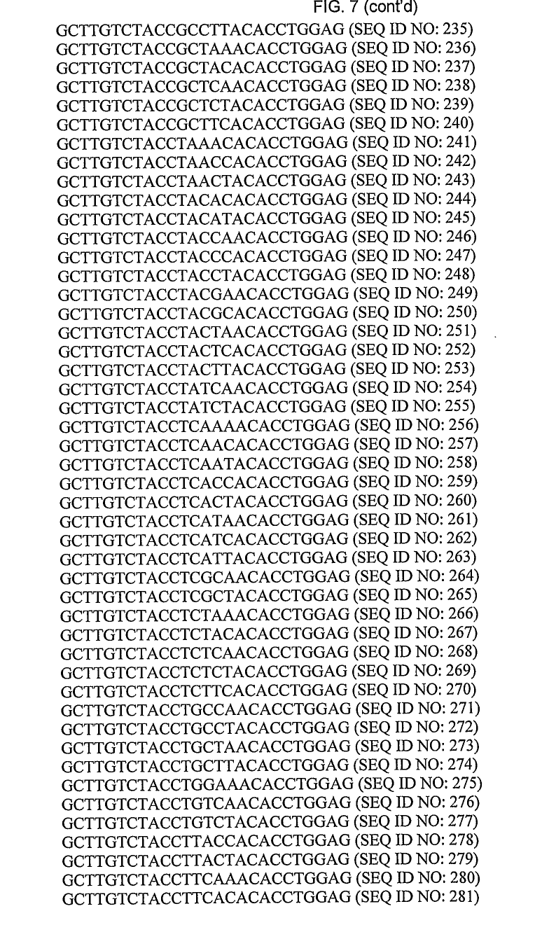

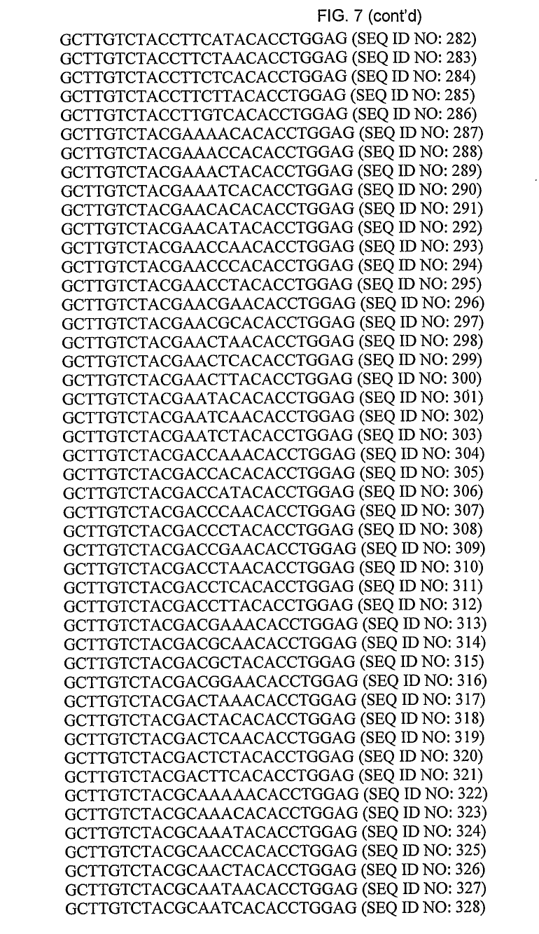

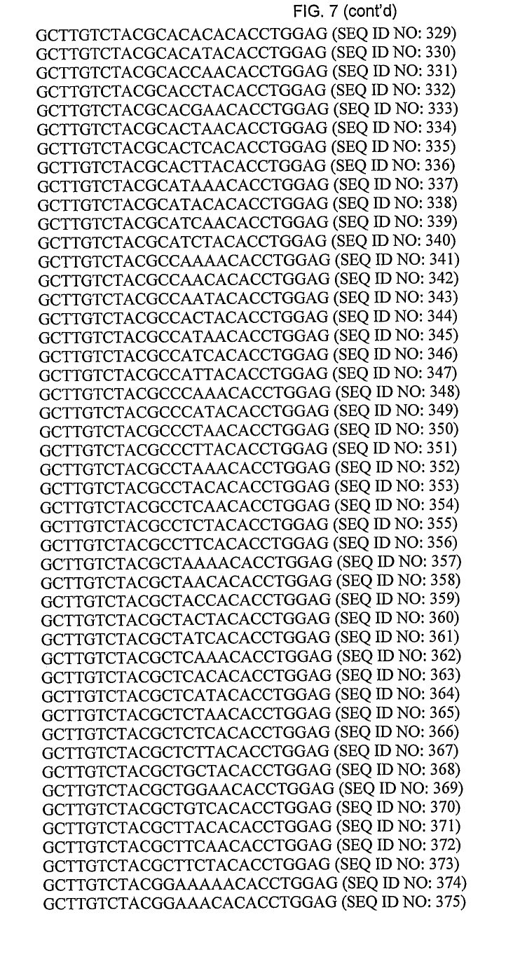

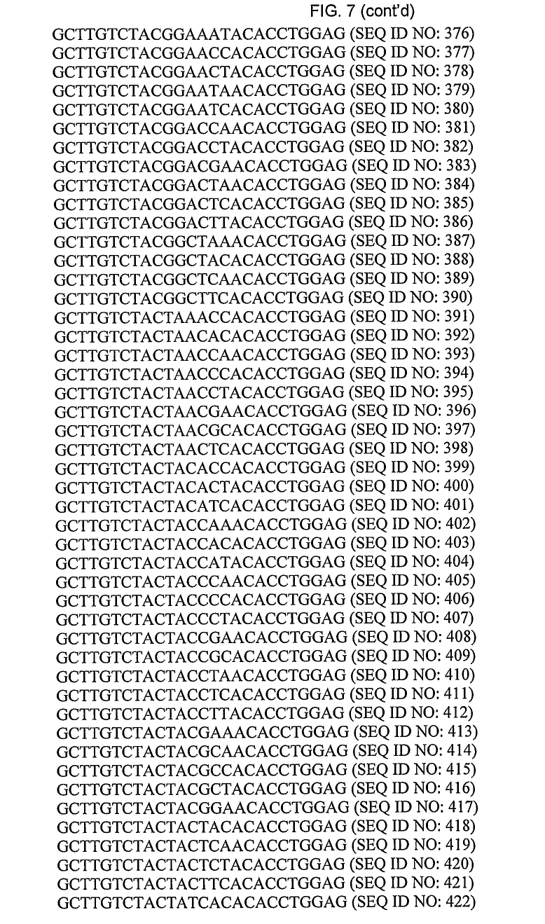

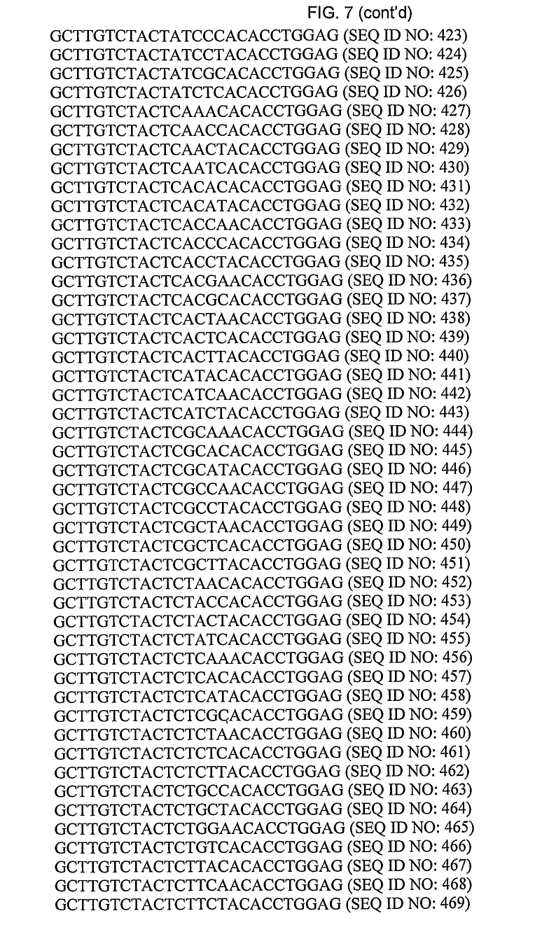

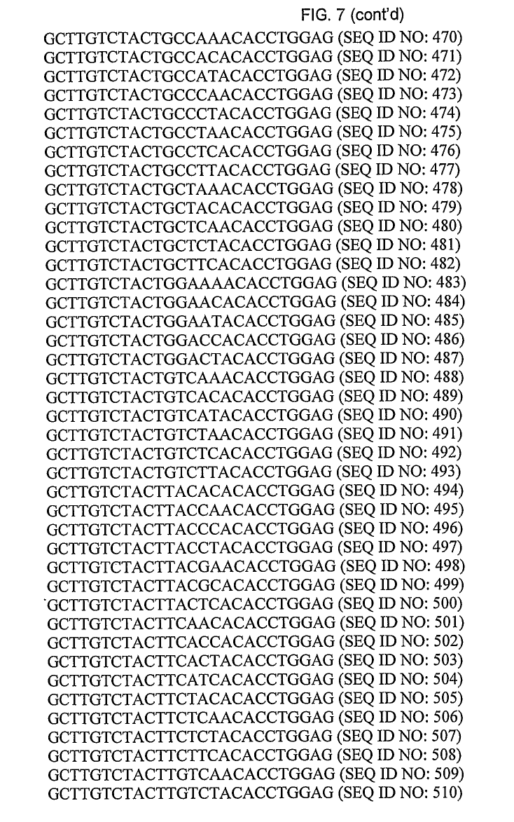

A set of exemplary oligonucleotide sequences useful in performing the present invention are set forth in FIG. 7. Other examples of codon systems and detailed discussions can be found in Examples and in U.S. Patent Application Publication Nos. 2004/0180412 A1 by Liu et al. and 2003/0113738 A1, by Liu et al.

Reagent strand sequences are designed according to the strategy described above for designing specificity domains in order to minimize the degree of interaction between reagent strands and minimize base-pairing between unintended reagent strand and template codons and anti-codons. In addition to the specificity domain design elements, reagents may also contain fixed flanking sequences of 2-10 bases that act as registration domains that insure proper orientation of the specificity domains with the template. Reagent strand sequences typically do not contain PCR primer binding sequences, and the target binding elements are attached through cleavable linkers to enable DPC.

Various constraints are placed in the selection of fragments. The fragments are selected with a bias by compiling a set of known ligands/drugs for a particular type of targets and generating a set of fragments from these starting points based on the constraints. Libraries of know ligands and drugs can be compiled or synthesized based on publicly available information and databases and are commercially available. Below in Table 1 are examples of constraints that may be used in selecting fragments for target binding elements.

TABLE 1
Examples of Fragment Selection Constraints

| | |
|---|---|
| Physical Property | Molecular Weight |
| constraints | ≦cLogP |
| | Number of Hydrogen Bond Donors (HBD) |
| | Number of Hydrogen Bond Acceptors (HBD) |
| | Polar Surface Area |
| | Total Surface Area |
| | Number of Rotatable Bonds |

The constraints may be adjusted in both reactive functional groups and physical properties. For example, the molecular weight of the fragments may be constrained to be more than 90, 100, 110, 120, 150 daltons and less than 500, 450, 400, 350, 300, 250, 200, 150 daltons. The values of c Log P can be between −2 and 4, 5, 6, 7, 8, 9, or 10, The numbers of HBD and HBA can be 1, 2, 3, 4, 5, 6, 7 or be set to be more or less than any of these numbers. Polar surface area preferably is <125 Å², more preferably <100 Å², 80 Å², or 60 Å²; total surface area preferably is <500 Å², more preferably <400 Å², 300 Å², 200 Å² or 100 Å²; the number of rotatable bonds preferably is <5, more preferably <4 or 3. Other properties may be used as constraints as well such as the number of chiral centers, e.g., one or none; two of fewer; three or fewer chiral centers, etc.

Additional constraints that may be applied to fragment selection or synthesis are presence of certain functional groups that may be useful attaching fragments to oligonucleotide strands, as shown by non-limiting examples in Table 2 below.

TABLE 2
Exemplary Functional Groups Useful to
Attach Fragments to Oligonucleotide

| Reactions | Functional Group Examples |
|---|---|
| 5′-Amino Reacts with: | —CO₂H; —COCl; —NCO; |
| | —NCS; —OCOCl; —CHO; |
| | —SO₂Cl |
| 5′-Amino Derivatized with Iodoacetyl | —SH |
| Reacts with: | |
| 5′-Thiol Reacts with: | COCH₂I, Acrylamide, |
| | Maleimide, Epoxide |
| 5′-Carboxy-NHS Ester Reacts with: | Amines |
| 5′-Hydroxyl: Mitsunobu Reaction with: | Phenols, Imides |
| 5′-Hydroxyl: Activation with Ms-Cl, | Amines, Thiols, Phenols, |
| Reacts with: | Imides, Stable carbanions |

A library of DPC-fragments can include any number of members depending on the synthetic methods used to make the library and on the target to be investigated. For example, the fragment library may contain 100 or less, 500; 1,000; 5,000; 10,000 or more members.

Exemplary target binding elements have been identified for a number of targets. See e.g., Erlanson, et al., 2004, J. Med. Chem., vol. 47(14), pp. 3463-3482; Fattori, 2004, Drug Disc. Today, vol. 9(5), pp. 229-239.

In one aspect, the invention provides a method for identifying a target binding element capable of binding to a binding domain disposed within a binding site of a target molecule. A target molecule is combined with a plurality of test molecules under conditions that permit a test molecule to bind to a binding domain of the target molecule. Each test molecule includes a target binding element that is associated with a corresponding oligonucleotide. The oligonucleotide has a nucleotide sequence that (i) identifies the target binding element, (ii) contains an amplification sequence, and (iii) is substantially incapable of hybridizing to the nucleotide sequence associated with other test molecules. A target binding element is harvested that binds to the target molecule with a K_{D} with a binding site greater than 10 μM. The sequence of the oligonucleotide associated with the target binding element harvested is determined so as to identify the target binding element that binds with a K_{D} of 10 mM or lower. In one embodiment, the oligonucleotide associated with the target binding element harvested is amplified. The sequence of the amplified oligonucleotide is determined so as to identify the target binding element that binds with a K_{D} of 10 mM or lower. In this method, each of substantially all of the target binding elements has at least one of the following characteristics: (i) a c Log P between −2 and 4, (ii) 4 or fewer H-bond donors, (iii) 8 or fewer H-bond acceptors, and (iv) a molecular weight between 90 and 500 daltons.

In another aspect, the invention provides a method for identifying a target binding element capable of binding to a binding domain disposed within a binding site of a target molecule. The target binding elements so identified have K_{D} values with the binding site greater than 10 μM. A target molecule is combined with a plurality of pre-selected test molecules under conditions that permit a test molecule to bind to a binding domain of the target molecule. Each test molecule includes a target binding element that is associated with an oligonucleotide. The oligonucleotide has a nucleotide sequence that (i) identifies the target binding element, (ii) contains an amplification sequence, and (iii) is substantially incapable of hybridizing (i.e., or does not hybridize) to the nucleotide sequences associated with other target binding elements. A target binding element is harvested that binds to the target molecule with a K_{D} greater than 10 μM. The oligonucleotide associated with the target binding element harvested is amplified. The sequence of the amplified oligonucleotide is determined so as to identify the target binding element having a K_{D} with the binding site greater than 10 μM.

In one embodiment, the method further includes the step of washing away unbound target binding elements after the combination of the plurality of pre-selected test molecules and harvest the target binding elements that bind to the target molecule with a pre-selected K_{D}, e.g., 1 μM, 10 μM, 20 μM, 50 μM or 100 μM. The method may further include washing away target binding elements that have a pre-selected K_{D} greater than, e.g., 50 μM, 100 μM, 200 μM, 500 μM, 1 mM, 100 mM, 500 mM or 1 M.

The target binding elements may have a mass ranging from 90 to 1,000 daltons. For example, the molecular weight of the target binding elements (e.g., fragments) may be constrained to be more than 90, 100, 110, 120, 150 daltons and less than 1,000, 500, 450, 400, 350, 300, 250, 200, or 150 daltons.

In one embodiment, the oligonucleotide is amplified by polymerase chain reaction wherein a primer anneals to the amplification sequence. A polymerase extends the primer annealed to the amplification sequence.

In yet another aspect, the invention provides an in vitro method for producing a molecule that binds to a pre-selected target molecule. The pre-selected target molecule includes a binding site that includes a first binding domain and a second binding domain. A template and a reagent are provided. The template includes a first target binding element attached to a first oligonucleotide that defines a first codon sequence. The first target binding element has a first K_{D} with the first binding domain of the binding site. The reagent includes a second target binding element attached to a second oligonucleotide that defines a first anti-codon sequence capable of hybridizing to the codon sequence. The second target binding element has a second K_{D} with the second binding domain. The template and the reagent are combined under conditions to permit the first codon sequence to hybridize to the first anti-codon sequence so as to bring the first and second target binding elements into reactive proximity. The first and second target binding elements are chemically coupled (e.g., in the absence of a ribosome) to produce a reaction product that has a K_{D} with the binding site less than (i) the first K_{D} of the first target binding element with the first binding domain, and (ii) the second K_{D} of the second target binding element with the second binding domain.

The method discussed here may include the step of selecting the reaction product. The method may further include the step of analyzing, e.g., by sequencing, the sequence of the first oligonucleotide associated with the reaction product. The sequence may also be determined by amplification. The sequence of the template is indicative of reaction product. The reaction product may include a first target element coupled to a plurality of second target elements.

In one embodiment, the first K_{D} of the first target binding element with the first binding domain is sufficient to permit the first target binding element to bind to the first binding domain in the absence of the second target binding element. In another embodiment, the first K_{D} of the first target binding element with the first binding domain is insufficient to permit the first target binding element to bind to the first binding domain in the absence of the second target binding element.

In another embodiment, the second K_{D} of the second target binding element with the second binding site is insufficient to permit the second target binding element to bind to the second binding domain in the absence of the first binding element.

In yet another embodiment, the first target binding element is known to bind to the first binding domain of the binding site. In one embodiment, the first target binding element is an anchor.

In one embodiment, the codon identifies the first target binding element associated with the first oligonucleotide. The anti-codon identifies the second target binding element associated with the second oligonucleotide. The template may include a plurality of different codons.

A plurality of different reagents may be combined with the template, and each reagent includes a different second target binding element attached to a corresponding, different oligonucleotide defining a corresponding anti-codon sequence. The anti-codon sequence is indicative of a particular second target binding element attached to the anti-codon.

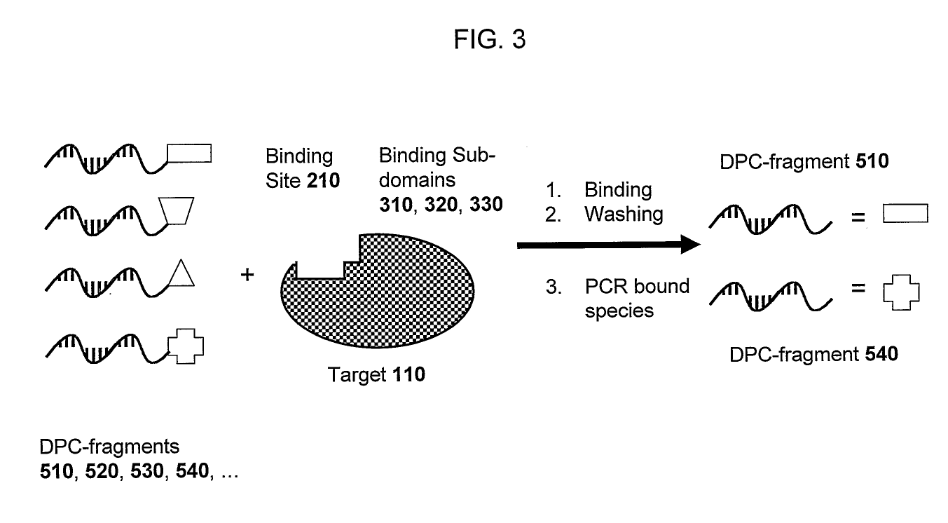

FIG. 3 schematically illustrates an exemplary method for the discovery of target binding elements that have binding affinities to a target. Target 110 having binding site 210 and domains 310, 320 and 330 is combined with DPC-fragments 510, 520, 530 and 540 having target binding elements 410, 420, 430 and 440, respectively. DPC-fragments 510 and 540 are harvested as they have the required binding characteristics (e.g., K_{D}). The corresponding oligonucleotide strands associated with 510 and 540 are amplified and deconvoluted to identify the DPC-fragments (revealing the identities of 510 and 540 which correspond to target binding elements 410 and 440).

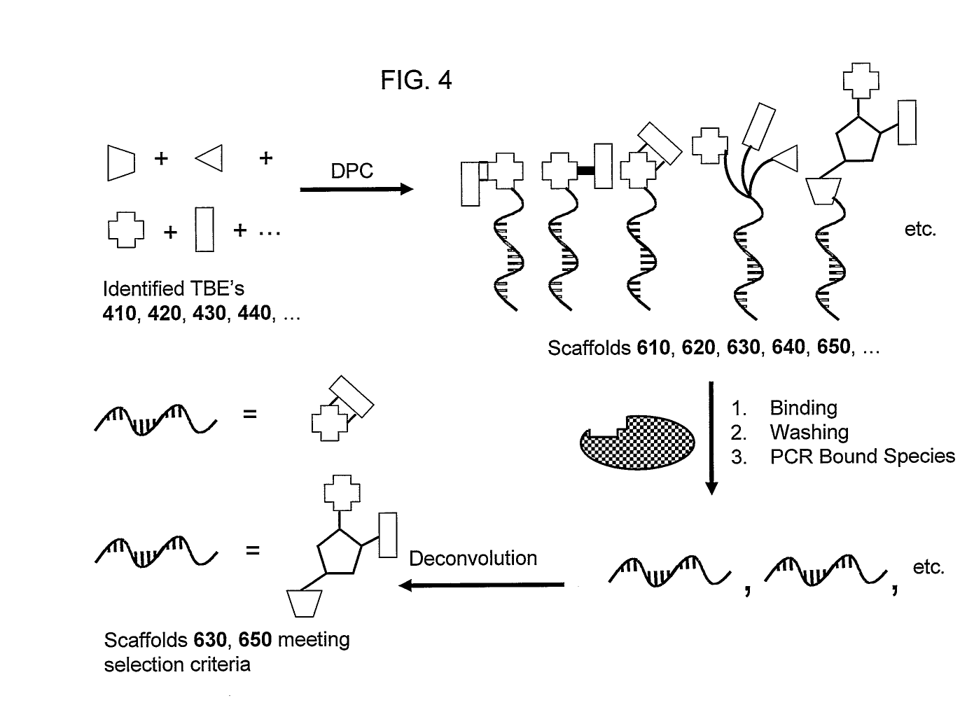

FIG. 4 is a schematic representation of an exemplary method for assembly and selection of target binding elements for a target and modular iteration to refine target binding. Identified target binding elements 410, 420, 430, 440, etc., are assembled (e.g., by DPC) to create scaffolds 610, 620, 630, 640, 650, etc. the assembly may be conducted under a pre-set criteria or randomly. The chemical assembly of the target binding elements can be accomplished using chemical methodologies that have been established as amenable to DPC. See, e.g., U.S. Patent Application Publication Nos. 2004/0180412 A1 and 2003/0113738 A1, Gartner et al., 2004, Science, 305(10), pp. 1601-1605; Liu, et al., 2002, Angew. Chem. Int. Ed., vol. 41(10), pp. 1796-2000). The TBE's can be linked directly to each other via covalent bonds or linker groups as shown for 610, 620, and 630 or they can be assembled using a scaffold. The scaffold can be flexible as in 640 or conformationally rigid as shown for 650.

The new target binding elements (i.e., scaffolds) 610, 620, 630, 640, 650, etc., are then subject to binding, oligonucleotide strand amplification and deconvolution so as to identify a subset of scaffolds that meet a certain binding characteristics (e.g., 630 and 650). More rounds of re-combination and selection or screening can be carried out to apply higher or different stringencies to optimize for binding, selectivity and other properties. Structural analogs of the TBE's can also be incorporated into the additional rounds of the process to expand the SAR of the interactions at the target binding domain(s).

Selection and/or screening for desired activities (e.g., binding affinity, catalytic activity, or a particular effect in an activity assay) may be performed according to any applicable protocol. See, e.g., U.S. Patent Application Publication Nos. 2004/0180412 A1 by Liu et al. and 2003/0113738 A1, by Liu et al.

For example, affinity selections may be performed according to the principles used in library-based selection methods such as phage display, polysome display, and mRNA-fusion protein displayed peptides. Selection for catalytic activity may be performed by affinity selections on transition-state analog affinity columns (see, e.g., Baca et al., 1997, Proc. Natl. Acad. Sci. USA 94(19): 10063-8) or by function-based selection schemes (see, Pedersen et al., 1998, Proc. Natl. Acad. Sci. USA 95(18): 10523-8). Since minute quantities of DNA (about 10⁻²⁰ mol) can be amplified by PCR (Kramer et al., 1999, Current Protocols IN Molecular Biology (ed. Ausubel, F. M.) 15.1-15.3, Wiley), these selections can be conducted on a scale ten or more orders of magnitude less than that required for reaction analysis by current methods. The selection strategy does not require any detailed structural information about the target molecule or about the molecules in the libraries.

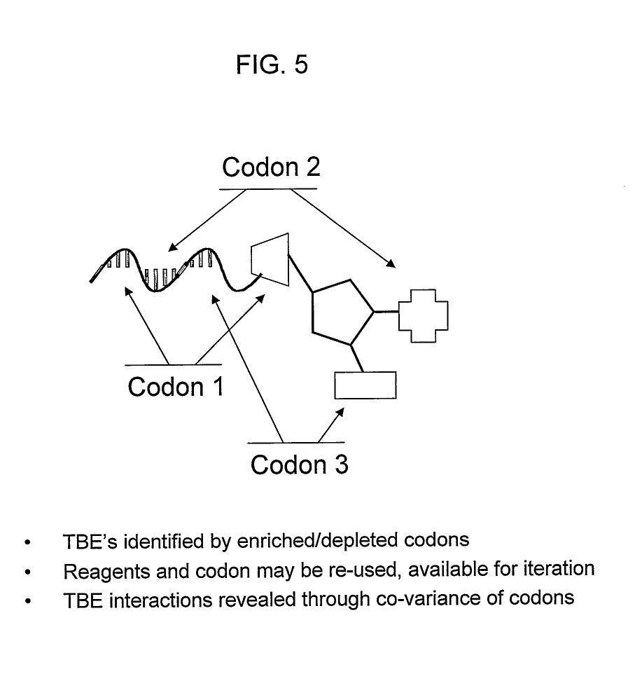

As schematically illustrated in FIG. 5, identification and selection of enriched and depleted target binding elements can be facilitated by the codons attached to the target binding elements.

In one embodiment, to allow deconvolution for DPC-fragments, DPC-fragments are designed to have only a single codon for identity, which renders the deconvolution process a relatively straight-forward analysis. Prior to a selection, the relative abundance of the various codons is determined by any of several methods, including real-time PCR (RT-PCR), microarray analysis, or single molecule sequencing. Following a selection, the same method is then applied, and the change in abundance of the DPC-fragment codons reveals enrichment or depletion. For real-time PCR, a unique set of primers for each DPC-fragment are employed, each in a single PCR reaction is designed to amplify a particular codon. The unique primers will typically be comprised of a common PCR primer sequence plus a primer that recognizes the unique codon. Monitoring the crossing-threshold of each uniquely amplified sequence reveals the relative abundance of each component. For microarray analysis, a microarray must first be generated that contains the various sequences that are complementary to the full set of DPC-fragment codons. Using a two-color system where, for example Cy-3 is used to identify pre-selection, Cy-5 is used for post-selection. The relative Cy-3:Cy-5 ratio reveals the degree of enrichment. For single molecule sequencing, the relative abundance of each individual codon is determined directly from the abundance of a given sequence in the mixture pre- and post-selection.

In one embodiment, to allow deconvolution for products of DPC library synthesis, the same set of techniques can be used to reveal enrichment or depletion of DPC templates due to selection of DPC library components. However, the analysis must take into consideration that each unique sequence is composed of three codons, and that each individual codon will find itself in the context of multiple unique template sequences. One preferred method for deconvolution involves simply determining by RT-PCR the enrichment at the codon level. Then, evaluation of intramolecular chemical interactions reveals by codon-codon covariance in the raw enrichment data to identify the preferred total structures. It is important to note that a single distribution of codon frequencies does not uniquely determine the distribution of DPC library components. Similar data can also be acquired by microarray, or single molecule sequencing as described above. With these other techniques, codon-codon covariance again reveals intramolecular chemical interactions.

In yet another aspect, the invention provides a composition that includes a plurality of test molecules. Each of substantially all of the test molecules includes a target binding element associated with a corresponding oligonucleotide. The oligonucleotide has a nucleotide sequence that (i) identifies the target binding element, (ii) contains an amplification sequence, and (iii) is substantially incapable of hybridizing to the nucleotide sequences associated with other target binding elements.

In one embodiment, each of at least some of the target binding elements has a K_{D} with a target binding site greater than 10 μM. In another embodiment, each of substantially all of the target binding elements has a K_{D} with a target binding site greater than 10 μM. In another embodiment, each of substantially all of the target binding elements has a molecular weight less than about 400 daltons.

In one embodiment, each of substantially all of the target binding elements is linked to a functional group through which the target binding element is attached to a corresponding oligonucleotide. Non-limiting examples of such functional groups include amines, carboxylic acids, acid chlorides, esters, ketenes, chloroformates, carbonates, aldehydes, acetals, thioacetals, ketones, ketals, thioketals, hydrazines, hydrazides, hydrazones, diazo compounds, esters, sulphonyl chlorides, alcohols, diols, phenols, azides, thiols, disulfides, isocyanates, isothiocyanates, alkyl and aryl halides, epoxides, aziridines, enamines, acrylamides, enones, maleimides, enolethers, imidates, oximes, nitrones, ylides, alkenes, dienes, and acetylenes.

In yet another aspect, the invention provides a composition that includes a plurality of test molecules. Each of at least some of the test molecules includes two or more target binding elements and is associated with a corresponding oligonucleotide. The oligonucleotide has a nucleotide sequence that (i) identifies the two or more target binding elements, (ii) contains an amplification sequence, and (iii) is substantially incapable of hybridizing to the nucleotide sequences associated with other test molecules.

In yet another aspect, the invention provides a composition that includes a plurality of test molecules. Each of substantially all of the test molecules includes two or more target binding elements and is associated with an oligonucleotide. The nucleotide has a nucleotide sequence that (i) identifies the two or more target binding elements, (ii) contains an amplification sequence, and (iii) is substantially incapable of hybridizing to the nucleotide sequences associated with other test molecules.

A test molecule may include 2, 3, 4, 5, 6 or more target binding elements. Test molecules may have various affinities toward a particular target, e.g., with a K_{D} to a target molecule less than 1 nM, 10 nM, 100 nM, 1 μM, 10 μM, 20 μM, 50 μM, 100 μM, 200 μM, 500 μM, 1 mM, 100 mM, 500 mM or 1 M or greater.

In yet another aspect, the invention provides a complex of a target molecule bound to a test molecule. The test molecule includes two or more target binding elements. The test molecule is associated with an oligonucleotide that has a nucleotide sequence that (i) identifies the test molecule and (ii) contains an amplification sequence. Each of substantially all of the target binding elements has at least one of the following characteristics: (i) a c Log P between −2 and 4, (ii) 4 or fewer H-bond donors, (iii) 8 or fewer H-bond acceptors, and (iv) a molecular weight between 90 and 500 daltons. As discussed herein, these and other constraints may be used to select target binding elements.

In yet another aspect, the invention provides a composition that includes a plurality of complexes. Each complex includes a target molecule bound to a test molecule. The test molecule includes two or more target binding elements, and each test molecule is associated with an oligonucleotide. The oligonucleotide has a nucleotide sequence that (i) identifies the test molecule, (ii) contains an amplification sequence, and (iii) is substantially incapable of hybridizing to the nucleotide sequence associated with other test molecules. Each of substantially all of the target binding elements is linked to a functional group through which the target binding element is attached to the oligonucleotide.

In yet another aspect, the invention provides a composition that includes a plurality of complexes. Each complex includes a target molecule bound to a test molecule that includes two or more target binding elements. Each test molecule is associated with an oligonucleotide that has a nucleotide sequence that (i) identifies the test molecule, (ii) contains an amplification sequence, and (iii) is substantially incapable of hybridizing to the nucleotide sequences of other test molecules.

The anchor-based approach of the present invention employs a ligand (e.g., a pharmacophore) that is known or found to bind to a target and use it as an anchor to assist other potential pharmacophores bind to known or unknown target binding sites. Particularly in the anchor-based approach, by incorporating an anchor moiety into the library (e.g., of scaffolds or fragments), the apparent binding affinity of weak binders to a target can be increased, thus allowing them to be identified through selections.

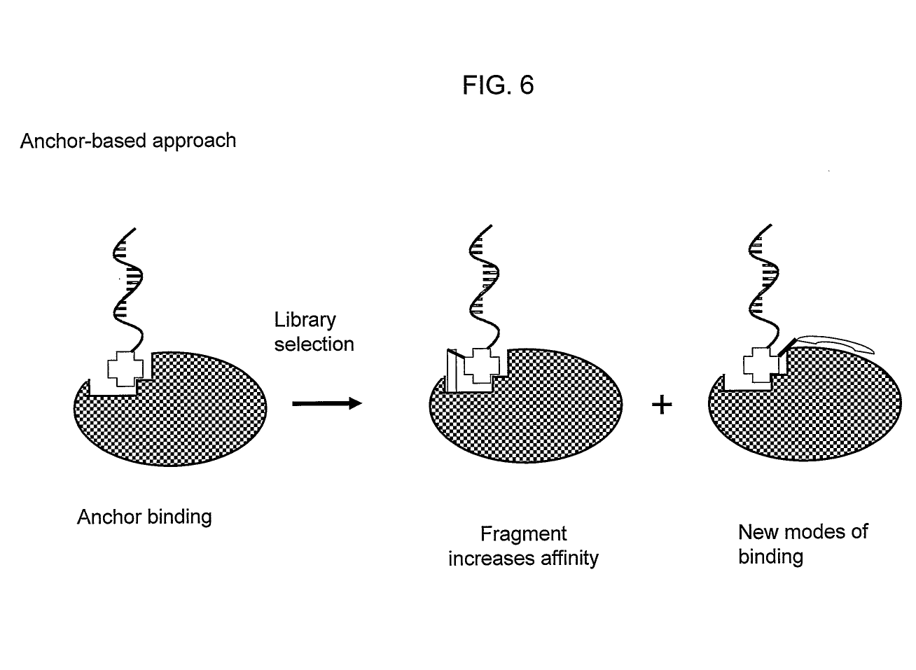

FIG. 6 is a schematic representation of one embodiment of anchor-assisted approach for identification of novel binding sites and generation of compounds having binding affinities to such binding sites. The fragments that are identified to have pre-selected binding properties can be optimized via a conventional medicinal chemistry approach, independent of amplifiable DNA conjugation, only if the method of observing binding is sufficiently sensitive to quantify weak interactions constituting an initial structure-activity relationship. Otherwise, the use of a high throughput ultra-sensitive DNA-dependent binding selection (e.g., Gartner et al., 2004, Science, vol. 305, pp1601-1605) is the method of choice. The latter method in conjunction with a DPC-based library approach, where one point of potential diversity on a particular scaffold is made invariant with the addition of a fragment, can be implemented. In this manner, the fragment serves as an “anchor” directing the library to the specific target of interest. Efficient selection of library members that bind more tightly to the target than the original anchor fragment alone provides a direct data-driven approach for lead optimization that is either independent of structural information for the target protein or can be complemented by it. Optimization of interactions distinct from the ones from the anchor fragment alone can lead to improved drug candidates analogous to the way the second generation ACE inhibitor, enalapril, was evolved from the first generation drug captopril.

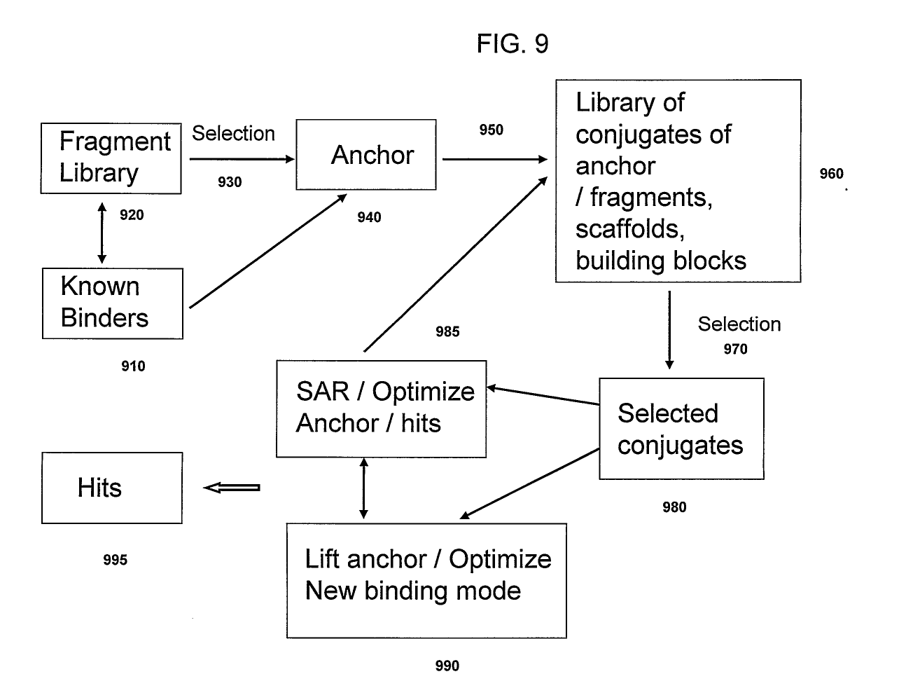

In one embodiment of the anchor-based approach, as illustrated schematically in FIG. 9, an anchor 930 is chosen from known binders 910 or from a fragment library 920 via selection 930. The anchor moiety is chemically incorporated (e.g., via DPC) at a point of diversity 950 in a library of compounds 960 (e.g., a diversity-oriented synthetic (DOS) DPC library) to generate an anchor-based subset of the original library (i.e., conjugates of the anchor moiety and the subset of the original library). A focused selection 970 is performed for the target of interest to which the selected anchor per se will bind to determine if positive selection is obtained for the members of the anchor-based subset (e.g., an anchor-based subset of the DOS DPC library). If positive selection is observed for the anchor-based subset resulting in a set of selected conjugates 980, the selection can be tuned by adding varying concentrations of the corresponding non-conjugated anchor. The optimal concentration of competing anchor can be determined empirically. The selection is considered optimized (tuned) 985 when the positive selection for the members of the anchor-based subset is lowered to its limit of quantitative detection. This completes the selection of anchor.

The anchor-based subset, used as the training set, can now be expanded 950 into a larger chemically diverse anchor-based library 960. The anchor moiety 940 (or an improved version) may now be incorporated into the larger library to generate an anchor-based library 960.

Next, a selection 970 as tuned above, can now be performed to identify binders from the newly expanded anchor-based library 960 with affinities greater than the anchor per se. The stringency of the selection can be increased to enable the elucidation of SAR by decreasing the concentration of the target protein or by further increasing the concentration of the competing anchor. The key point is that the higher affinity of certain library members will result from interactions at positions of diversity distinct from the anchor moiety.

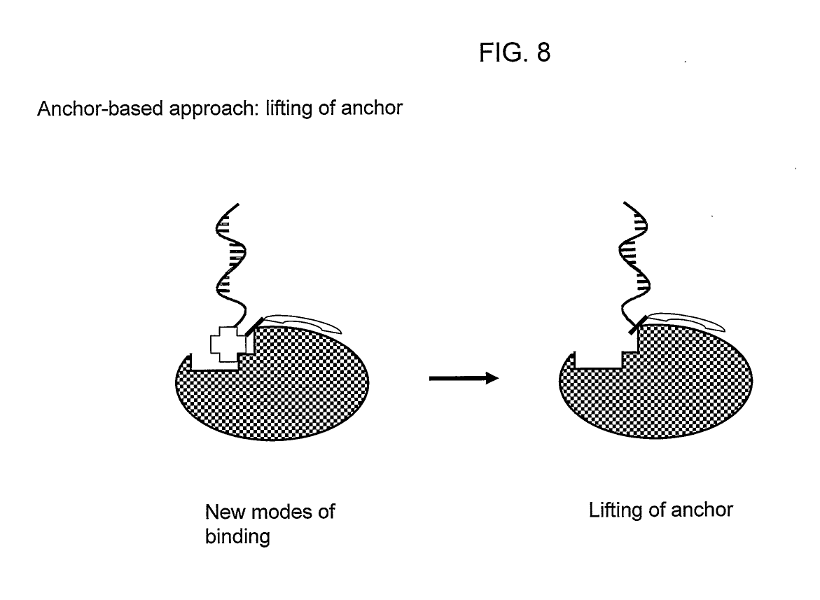

The resulting SAR from the above selection of the expanded library can be used in the design of follow-up libraries. The above process may be iterated, and optimization of binding through this iterative process will enable the exploration of both novel chemical and biological space distinct from the original anchor moiety and its binding site on the target. In certain cases it may be appropriate to remove (lift) the original anchor moiety, allowing a closer study of new modes of binding and binding sites potentially addressing issues related to selectivity and other properties (e.g., mechanism-based and non-mechanism-based toxicity). See FIG. 8.

The anchor-based example above illustrates the use of an anchor to explore the target topology adjacent to the anchor binding site and to identify potentially new binding domains and small molecule pharmacophores for these domains. The anchor approach described herein does not require a covalent bond be formed between the anchor and the target of interest (i.e., without “tethering”). Thus, no structural knowledge about the target is necessary. This approach is complementary to the fragment approach disclosed herein that seeks to identify small molecules that bind with weak affinity to targets. One advantage of the invention is that it allows the anchor to direct pharmacophore exploration to a region of the target that has been shown to produce desired therapeutic effects through ligand binding. Binding of a ligand to a target in itself may be insufficient for a therapeutic effect; however, binding of a ligand to a target domain that elicits a desired therapeutic effect has a higher probability of success in drug discovery. This method enables a discovery platform that tightly and efficiently integrates chemistry and biology providing a direct means to identify totally novel structures with corresponding novel modes of binding action from known chemical and biological space.

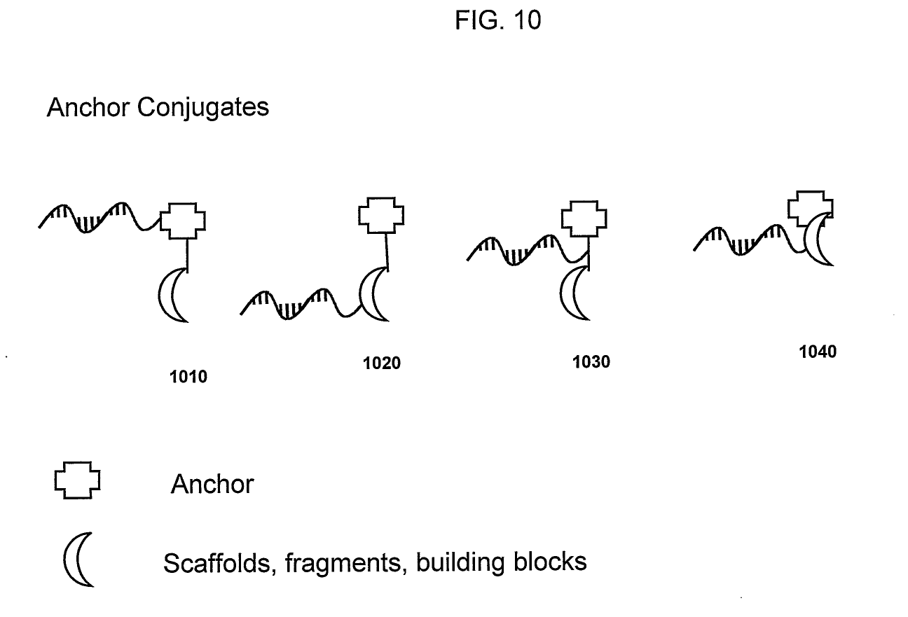

The anchor-based approach may be implemented in various ways, as schematically illustrated in FIG. 10. In one approach 1010, the oligonucleotide is linked directly to the anchor and not directly linked to the scaffold (or fragment or building blocks). As an example, Phg-Arylsulfonamide may be employed as an anchor to direct a macrocyclicfumaramide (MCF) library to the active site of carbonic anhydrase. In another approach 1020, the oligonucleotide is directly linked to the scaffold (or fragment or building blocks) and not directly linked to the anchor. In yet another approach 1030, the oligonucleotide is indirectly linked to both the anchor and the scaffold (or fragment or building blocks).

In another approach 1040, the anchor may be an integral part of the scaffold and actually remains a part of the final optimized compound. In this approach, the anchor still functions to direct the fragment or scaffold to a binding domain of the target but also serves as an integral component of the resulting pharmacophore and continues in the iterative library process to yield the optimized moiety.

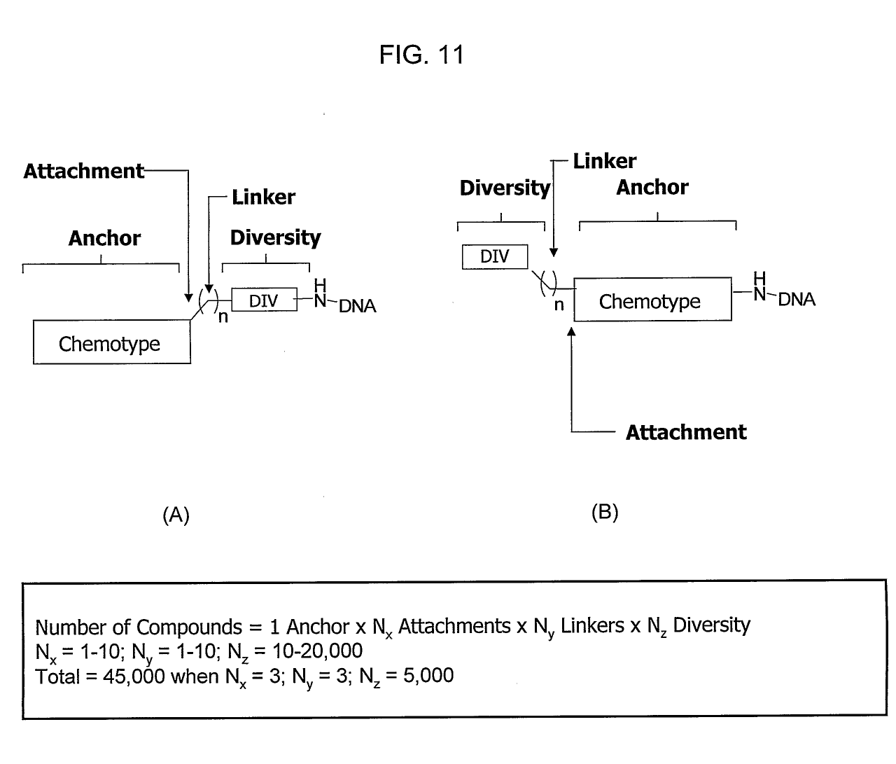

FIG. 11 illustrates exemplary architects of anchor libraries. FIGS. 11(A) and (B) show two alternative approaches in linking the anchor moiety and the diversity portion of the anchored compound. The total number of compounds may be controlled by the numbers of the anchor, attachment points, linkers, diversity building blocks, etc. Crystalline structures of the anchor and the target where available may be helpful in designing a library of compounds to address a particular target.

As an example of this approach, statine residues may be incorporated into a MCF library (see, e.g., U.S. Patent Application Publication Nos. 2004/0180412 A1 by Liu et al. and 2003/0113738 A1, by Liu et al.), FIG. 12. In this case, statine is a known moiety that can bind to the catalytic site of aspartyl proteases. By incorporating this residue into the MCF library at either R1, R2, or R3, the catalytic machinery is targeted with a known pharmacophore (anchor) and MCF members with appropriate topology for binding may be identified. In subsequent DPC library iterations, the anchor will remain and may also be optimized along with R2 and R3 (i.e. side chain diversity of statine). Although the statine residue may undergo structural changes in the optimization process, the overall topology of the MCF scaffold will remain intact and the modified anchor will be a part of the optimized molecules.

In one aspect, the invention provides a method for selecting a compound having a desired binding affinity to a target molecule. The method includes the following. A library is provided that includes a plurality of test compounds. Each of the test compounds includes (1) a common binding moiety, (2) a scaffold moiety connected to the common binding moiety through a bridging moiety, and (3) an oligonucleotide having a nucleotide sequence informative of the structural or synthetic information of the associated test compound. The common binding moiety has a dissociation constant of 10 mM or lower to a first binding domain of the target molecule. A reference compound is provided that includes the common binding moiety. The target molecule, the plurality of test compounds, and the reference compound are combined under conditions that permit the plurality of test compounds and the reference compound to compete for binding to the target molecule. The test compounds that exhibit greater binding affinity to the target molecule than the reference compound are harvested. The oligonucleotide sequences of the test compounds harvested are determined thereby to identify the test compounds having a desired binding affinity to the target molecule.

In another aspect, the invention provides a method for identifying a compound having a desired binding affinity to a target molecule. The method includes the following. The target molecule, a plurality of test compounds, and a reference compound are combined under conditions that permit the plurality of test compounds and the reference compound to compete for binding to the target molecule. Each of the plurality of test compounds includes (1) a common binding moiety, (2) a scaffold moiety connected to the common binding moiety through a bridging moiety, and (3) an oligonucleotide having a nucleotide sequence informative of the structure or synthetic information of the associated test compound. The reference compound includes the common binding moiety. The common binding moiety has a dissociation constant of 10 mM or lower to a first binding domain of the target molecule. The oligonucleotide sequences of the test compounds that bound to the target are determined.

In yet another aspect, the invention provides a library of chemical compounds. The library includes a plurality of compounds. The compounds are prepared by one or more nucleic-acid-templated chemical reactions. Each of the compounds comprises (1) a first moiety, (2) a second moiety connected to the first moiety through a bridging moiety, and (3) an oligonucleotide having a nucleotide sequence informative of the structure or synthetic information of the second moiety. The first moiety has a dissociation constant of 10 mM or lower to a binding domain of the target molecule.

In yet another aspect, the invention provides a method for detecting a second binding domain on a target molecule having a first binding domain. The method includes the following. A test compound is provided that includes (1) a first binding moiety having a binding affinity to the first binding domain of the target molecule, (2) a scaffold moiety connected to the first binding moiety through a bridging moiety, and (3) a defining oligonucleotide having a nucleotide sequence informative of the structure or synthetic information of the test compound. The first binding moiety has a dissociation constant of 10 mM or lower to a first binding domain of the target molecule. The effect of the test compound on the binding of a reference compound to the target molecule is determined. The reference compound comprises the first binding moiety. The data collected is analyzed to detect the presence of a second binding domain on the target molecule.

In yet another aspect, the invention provides a method for identifying a compound having a desired binding affinity to a target molecule. The method provides the following. A library is provided that includes a plurality of test compounds, wherein each of the test compound comprises (1) a common binding moiety, (2) a scaffold moiety connected to the common binding moiety through a bridging moiety, and (3) an oligonucleotide having a nucleotide sequence informative of the structural or synthetic information of the associated test compound. The common binding moiety has a dissociation constant of 10 mM or lower to a first binding domain of the target molecule. The target molecule and the plurality of test compound are combined under conditions that permit binding of one or more of the plurality of test compounds to the target molecule if such test compounds with desired binding affinity are present. The test compounds bound to the target are harvested. The oligonucleotide sequences of the test compounds harvested are determined thereby identifying the test compounds having a desired binding affinity to the target molecule.

In yet another aspect, the invention provides a method for selecting a compound having a desired binding affinity to a target molecule. The method includes the following. A library is provided that includes two subsets of test compounds. Each of the first subset of test compounds includes (1) a common binding moiety, (2) a first scaffold moiety connected to the common binding moiety through a bridging moiety, and (3) an oligonucleotide having a nucleotide sequence informative of the structural or synthetic information of the associated test compound. The common binding moiety has a dissociation constant of 10 mM or lower to a first binding domain of the target molecule. Each of the second subset of test compounds includes (1) a second scaffold moiety, and (2) an oligonucleotide having a nucleotide sequence informative of the structural or synthetic information of the associated test compound. The first scaffold and the second scaffold may be the same scaffold. A reference compound is provided that includes the common binding moiety. The target molecule, the library of test compounds, and the reference compound are combined under conditions that permit the plurality of test compounds and the reference compound to compete for binding to the target molecule. The test compounds that exhibit greater binding affinity to the target molecule than the reference compound are harvested. The oligonucleotide sequences of the test compounds harvested are determined thereby to identify the test compounds having a desired binding affinity to the target molecule.

In yet another aspect, the invention provides a composition that includes a plurality of test molecules. Each of at least some of the test molecules includes two or more target binding elements and is associated with a corresponding oligonucleotide. The oligonucleotide has a nucleotide sequence that (i) identifies the two or more target binding elements, (ii) contains an amplification sequence, and (iii) is substantially incapable of hybridizing to the nucleotide sequences associated with other test molecules.

In yet another aspect, the invention provides a composition that includes a plurality of test molecules. Each of substantially all of the test molecules includes two or more target binding elements and is associated with an oligonucleotide. The nucleotide has a nucleotide sequence that (i) identifies the two or more target binding elements, (ii) contains an amplification sequence, and (iii) is substantially incapable of hybridizing to the nucleotide sequences associated with other test molecules.

In yet another aspect, the invention provides a compound. The compound comprises (1) a first moiety, (2) a second moiety connected to the first moiety through a bridging moiety, and (3) an oligonucleotide having a nucleotide sequence informative of the structure or synthetic information of the second moiety. The first moiety has a dissociation constant of 10 mM or lower less to a binding domain of the target molecule.

The following examples contain important additional information, exemplification and guidance that can be adapted to the practice of this invention in its various embodiments and equivalents thereof. Practice of the invention will be more fully understood from these following examples, which are presented herein for illustrative purpose only, and should not be construed as limiting in anyway.

### EXAMPLES

### Example 1

### Exemplary Anchors

Examples of anchors are shown in the following tables. Tables 3 and 4 is a set of anchors that may be utilized for targets in the Carbonic Anhydrase class. Anchors for targets in the Kinase class, particularly BCR/Abl and VEGFR2, are shown in Table 5. Anchors for phosphatase targets, particularly PTP1b, are shown in Table 6. These anchors can be prepared according to the general protocols described below;

TABLE 3
Carbonic Anhydrase Anchors: 5′-DNA Conjugates

| | | | | M⁻¹ Digest |
|---|---|---|---|---|
| Anchor | Linker | Codons | Sequence | (Calculated) |
| 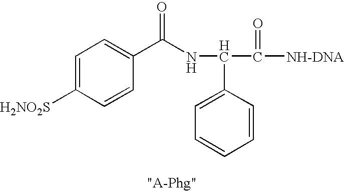 | 5′-Amino-5 | 1-60-101 | CAGACGTCA CGCCAAACT CACTACCAG CACTCTTCCG TCCACTACAAC (SEQ ID NO: 511) | 709.1407 (709.1693) |
| 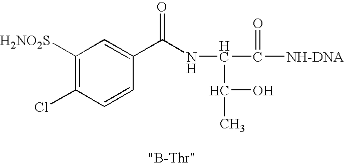 | 5′-Amino-5 | 29-60-101 | CAGACGTCA CCAGAACCT CACTACCAG CACTCTTCCG TCCACTACAAC (SEQ ID NO: 512) | 711.1437 (711.1253) |
| 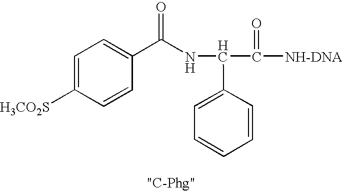 | 5′-Amino-5 | 93-60-101 | CAGACGTCA CAAGCCTCT CACTACCAG CACTCTTCCG TCCACTACAAC (SEQ ID NO: 513) | 708.1271 (708.1741) |
| 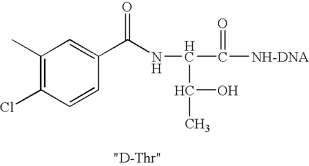 | 5′-Amino 5 | 94-60-101 | CAGACGTCA CTGTCCTCTC ACTACCAGC ACTCTTCCGT CCACTACAAC (SEQ ID NO: 514) | 646.1626 (646.1681) |

TABLE 4
Carbonic Anhydrase Anchors: 3′-DNA Conjugates

| | | | | M⁻¹ Digest |
|---|---|---|---|---|
| Anchor | Linker | Codons | Sequence | (Calculated) |
| 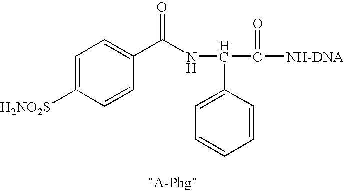 | 3′-Amino C7 | T40-6-45-85 | CCACTACAA CACATCCCTC ACCCGTAAC ACTCCTTAGC CTCACCGCA ATCGAATTCCAC (SEQ ID NO: 515) | 542.12 (542.14) |

TABLE 5
Kinase Anchors: 3′-DNA Conjugates

| | | | | M⁻¹ Digest |
|---|---|---|---|---|
| Anchor | Linker | Codons | Sequence | (Calculated) |
| 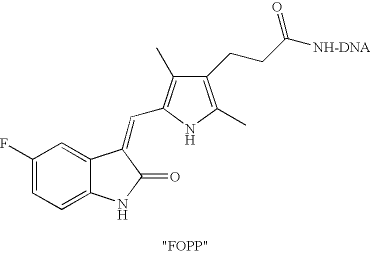 | (mini-PEG2)- (miniPEG3)- 3′-Amino C7 | T40-3- 42-65 | CCACTACAACA CATCCCTCACC GTCAACACTCT ACAGCCTCACC GCAATCGAATT CCAC (SEQ ID NO: 516) | 870.3602 (870.87) |
| 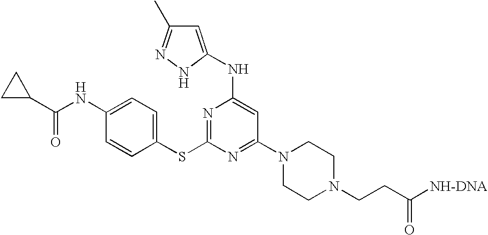 | (mini-PEG2)- (miniPEG3)- 3′-Amino C7 | T40-25- 56-109 | CCACTACAACA CATCCCTCACC TCCTACACTCG CTTTCCTCACG ACCTTCGAATT CCAC (SEQ ID NO: 517) | 1064.5043 (1064.47) |

TABLE 6
Phosphatase Anchors: 3′-DNA Conjugates

| | | | | MWt |
|---|---|---|---|---|
| Anchor | Linker | Codons | Sequence | (Full Length) |
| 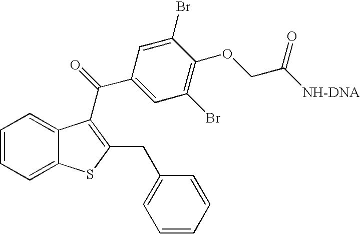 | 3′-Amino C7 | T40-25-58-85 | CCACTACAA CACATCCCTC ACCTCCTAC ACTCCCTAA GCTCACCGC AATCGAATT CCAC (SEQ ID NO: 518) | (M-10)⁻¹⁰ = 1843.4909 |
| 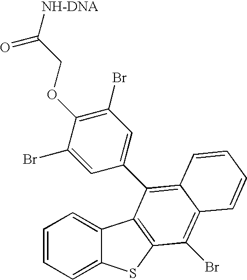 | 3′-Amino C7 | T40-25-58-86 | CCACTACAA CACATCCCTC ACCTCCTAC ACTCCCTAA GCTCACCTG CATCGAATT CCAC (SEQ ID NO: 519) | (M-10)⁻¹⁰ = 1848.6775 |
| 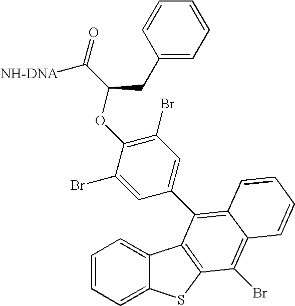 | 3′-Amino C7 | T40-25-58-87 | CCACTACAA CACATCCCTC ACCTCCTAC ACTCCCTAA GCTCACGCT CATCGAATT CCAC (SEQ ID NO: 520) | (M-11)⁻¹¹ = 1688.7175 |

DNA oligonucleotides were synthesized on a PerSeptive Biosystems Expedite 8090 DNA synthesizer using standard phosphoramidite protocols and purified by reverse phase HPLC with a triethylammonium acetate/acetonitrile gradient. The 5′-amino modified oligo nucleotides were prepared by standard automated DNA synthesis using the 5′-amino-modifier 5 phosphoramidite from Glen Research. The 3′-amine modified oligonucleotides were prepared with the same protocol but with the 3′-amino-modifier C7 CPG from Glen Research. 3′-biotin oligonucleotides were prepared using Biotin TEG CPG from Glen Research.

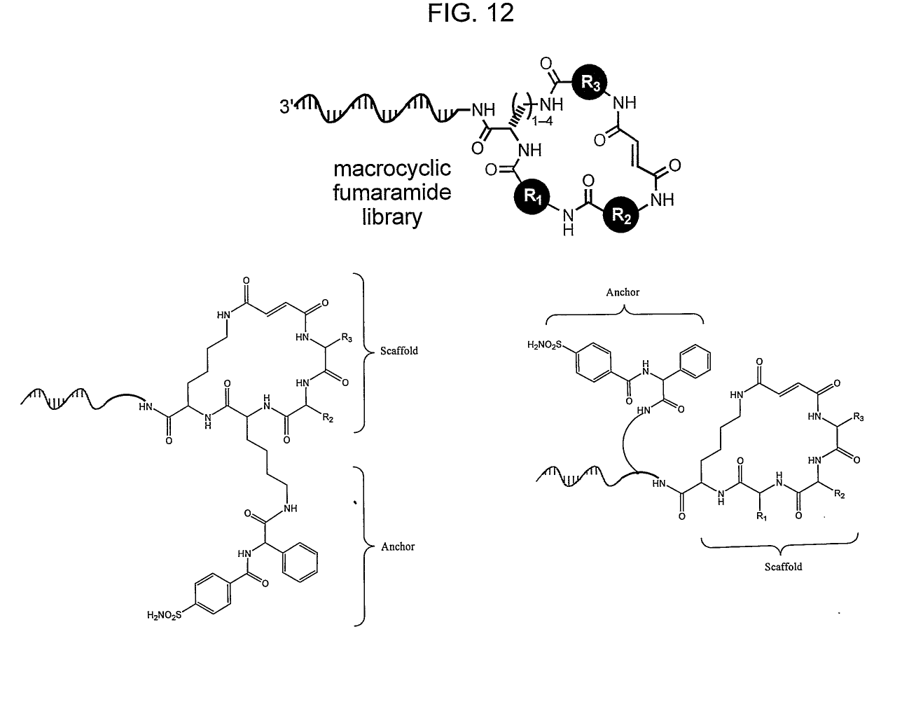

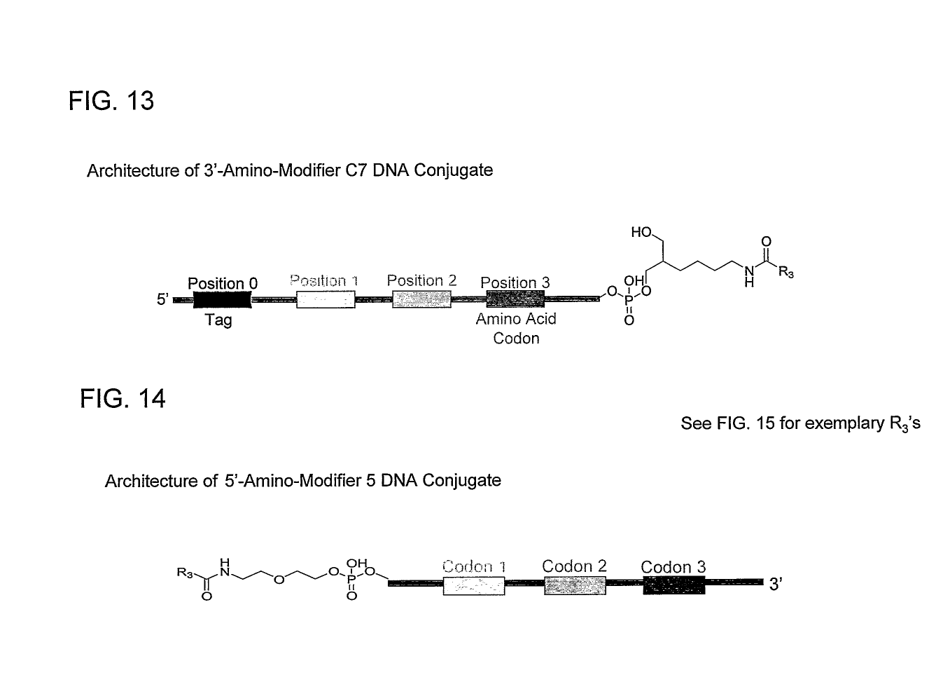

To prepare the 5′-amine or 3′-amine modified anchored DNA strands, the oligonucleotides were prepared by standard automated DNA synthesis. The oligonucleotides were purified by RP-HPLC prior to conjugation to the various Anchor molecules. The general architectures of the 3′-amine or 5′-amine modified DNA strands are shown in FIG. 13 and FIG. 14, respectively.

The anchor molecules as carboxylic acids were converted to the N-hydroxysuccinimide active esters, which were then conjugated to the 5′-amino or 3′-amino-modified oligos according to the following general protocols.

General protocol for preparing O-succinimidyl (OSu) ester: Free acid (0.5 mmole, 1 equiv.) and N-hydroxy succimide (0.6 mmole, 1.2 equiv.) were dissolved in 1.7 mL of anhydrous DMF under Ar, then N,N′-dicyclohexylcarbodiimide (DCC, 0.5 mmole, 1 equiv.) in 0.8 mL of anhydrous DMF were added (final concentration for free acid is 0.2 M). The reaction mixture was stirred at 40° C. for one to four hours. The extent of the OSu ester formation can be monitored by TLC. The presence of small amount of free acid can be neglected. After the reaction mixture was cooled in refrigerator (2 to 8° C.) for several hours, the precipitated dicyclohexylurea (DCU) was then removed by filtration. The filtrate was treated with 15 mL of ether. Solid precipitated was washed three times with 10 mL of ether and dried under vacuum for several hours to afford the desired product (yield rang from 70 to 100%).

General protocol for derivatizing DNA using OSu ester: To a 1.5 mL of centrifugation vial containing 50 nmole of DNA was added 104 μL of 0.1 M sodium phosphate buffer (NaPi), pH 8.6, 104 μL of OSu ester in NMP (96 mM or 72 mM) and 104 μL of NMP (final concentration for DNA: 0.16 mM). The vial was placed in a shaker and shaked at 37° C. for 1 hr to overnight. The extent of the DNA labeling can be monitored by analytical HPLC. The reaction mixture was desalted by gel filtration using Sephadex G-25 and then further purified by semi-preparative reversed-phase C18 column.

### Example 2

### Exemplary Fragments

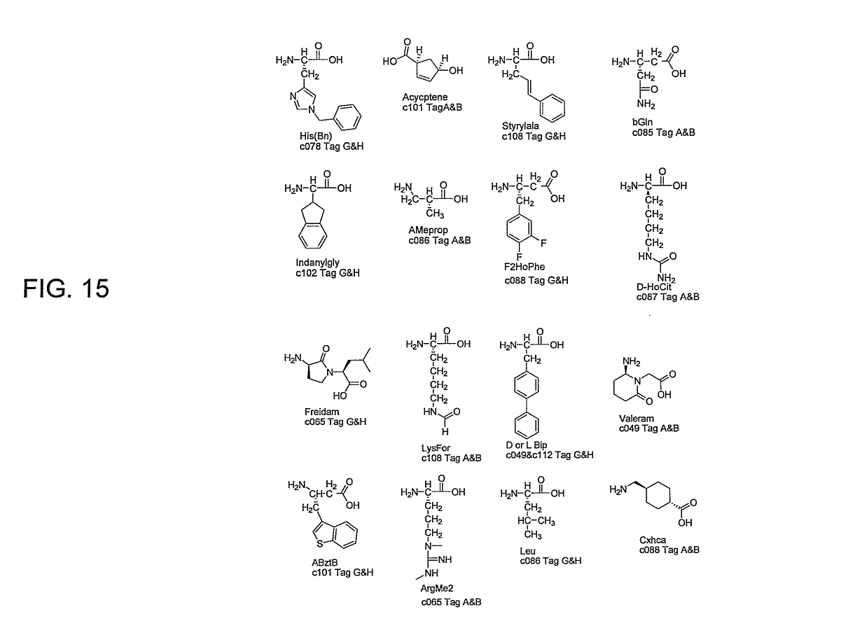

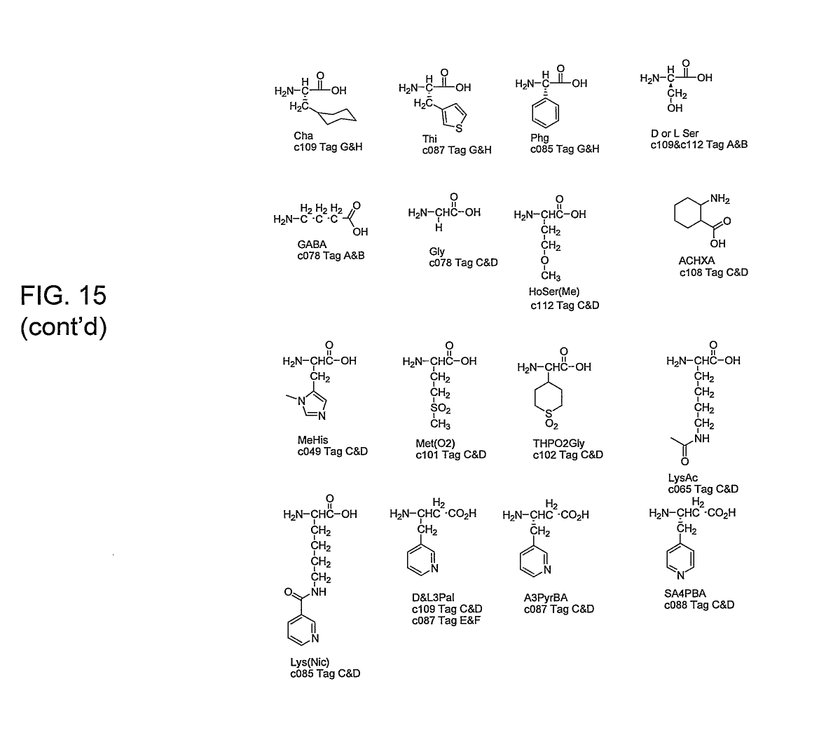

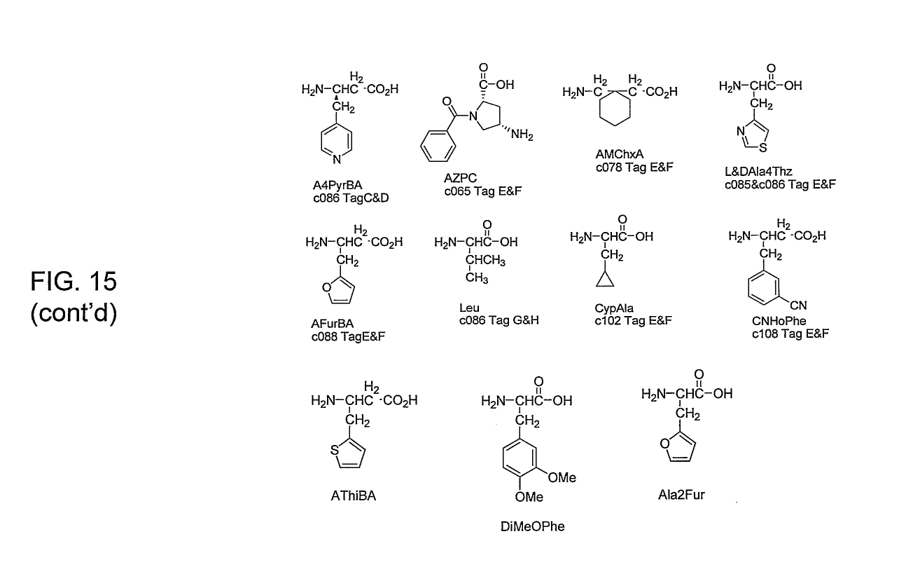

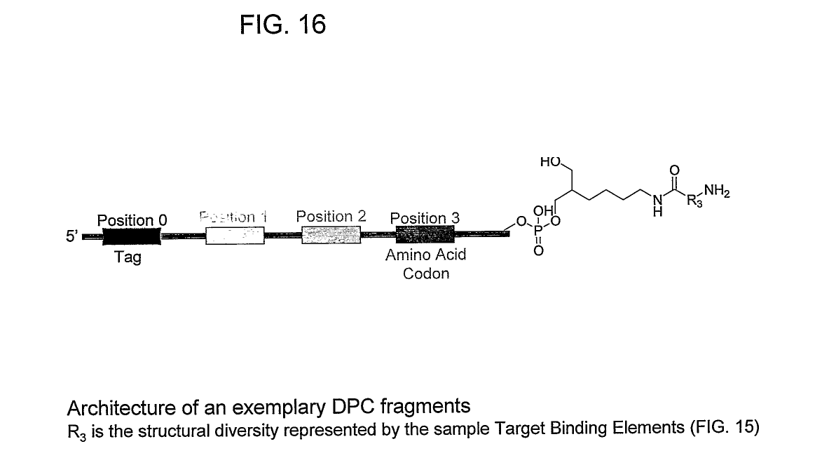

A set of fragments (see FIG. 15) are chosen according to the constraints of Table 7 below and modified as needed. The fragments are selected with a bias by compiling a set of known ligands/drugs for BCR/Abl and related kinases and generating a set of fragments from these starting points based on the constraints of Table 7. Libraries of know ligands and drugs can be compiled from publicly available information and databases and are commercially available.

TABLE 7
Examples of Fragment Selection Constraints

| | Exemplary Constraints A | Exemplary Constraints B |
|---|---|---|
| Reactive | Primary and secondary | Primary and secondary |
| functional | amines | amines |
| groups: | Primary anilines | Primary anilines |
| | Carboxylic acids | Carboxylic acids |
| | Bifunctional reagents | Bifunctional reagents |
| | containing an amine and | containing an amine and |
| | a carboxylic acid moiety | a carboxylic acid moiety |
| Physical | 90 < Molecular Weight < 500 | 90 < Molecular Weight < 300 |
| Property | −2 ≦ cLogP ≦ 4 | −2 ≦ cLogP ≦ 4 |
| constraints | Hydrogen Bond Donors ≦ 4 | Hydrogen Bond Donors ≦ 3 |
| | Hydrogen Bond | Hydrogen Bond |
| | Acceptors ≦ 8 | Acceptors ≦ 6 |

The constraints may be adjusted in both reactive functional groups and physical properties. For example, the molecular weight of the fragments may be constrained to be more than 90, 100, 110, 120, 150 daltons and less than 500, 450, 400, 350, 300, 250, 200 or 150 daltons. The values of c Log P can be between −2 and 4, 5, 6, 7, 8, 9, or 10. The numbers of HBD and HBA can be 1, 2, 3, 4, 5, 6, 7 or be set to be more or less than any of these numbers. Other properties may be used as constraints as well such as the number of chiral centers, e.g., one or non; two of fewer; three or fewer, etc.; The number of NO₂ groups, e.g., 0, 1, 2, 3, 4, or more or less than any of these numbers. Additionally, the polar surface area, the total surface area, and the number of rotatable bonds may be used to define and select fragments.

### Example 3

### Fragment-Based DPC Discovery

Each of the fragments is coupled to a specific DNA detection strand or reagent strand, and purified according to standard methods. There are many methods available to one skilled in the art for coupling strands to TBE's. Methods and references to these procedures can be readily obtained from many advanced text in organic chemistry, such as Carey, F. A. and Sundberg, R. J., Advanced Organic Chemistry Fourth Edition, Parts A & B, Kluwer Academic/Plenum Publishers, 2000; or March, Advanced Organic chemistry, John Wiley & Sons, New York, Fourth Edition, 1992. Non-limiting exemplary linkages include: amides (e.g., Carey et al. Part B, pp. 172-179); ureas (e.g., March, pp. 1299), carbamates (e.g., March, pp. 1280), sulfonamides (e.g., March, pp. 1296), aminoalkyl via reductive amination of amines with aldehydes or ketones (e.g., Carey et al., pp. Part B. pp. 269-270), thioethers (e.g., Carey et al., pp. 158; March, pp. 1297), ethers via Mitusunobu (e.g., Carey et al., pp. 153-154), and carbon-carbon bonds via carbanions (e.g., Carey et al., pp. 39-47) Purification of the DPC fragments can be accomplished by a number of methods available to those skilled in the art, such as but not limited to reverse phase HPLC, ion exchange chromatography and electrophoresis.

### Preparation of Sample DPC Fragments

DNA oligonucleotides were synthesized on a PerSeptive Biosystems Expedite 8090 DNA synthesizer using standard phosphoramidite protocols and purified by reverse phase HPLC with a triethylammonium acetate/acetonitrile gradient. The 5′-amino modified oligo nucleotides were prepared by standard automated DNA synthesis using the 5′-amino-modifier 5 phosphoramidite from Glen Research. 5′-Thiol oligonucleotides were obtained with the 5′-Thiol-Modifier C6 from Glen Research. The 3′-amine modified oligonucleotides were prepared with the same protocol but with the 3′-amino-modifier C7 Controlled Pore Glass (CPG) from Glen Research. 3′-biotin oligonucleotides were prepared using Biotin Triethyleneglycol (TEG) CPG from Glen Research.

To prepare the 3′-amine modified DPC fragments, the Fmoc-amine protected Target Binding Elements shown in FIG. 15 were coupled to the 3′-amino-modifier C7 CPG using standard coupling protocols for peptide synthesis (Carey, F. A. and Sundberg, R. J., Advanced Organic Chemistry Fourth Edition, Part B, pp. 172-179). The oligonucleotides were then prepared by standard automated DNA synthesis. The architecture of the DPC Fragments is shown in FIG. 16. The 3-amino-modifier C7 is shown linking the Fragment to the 3′ end of the DNA strand. From 3′ to 5′, the sequence consists of a PCR primer region, followed by the Position 3 codon that identifies the fragment. The position 2 and 1 codons follow and are available for templating DPC with complementary reagent strands. Position 0 represents a codon that uniquely identifies each sub-pool such that re-use of codons at positions 1-3 in different tag pools is enabled. The 5′-terminus is a PCR primer region.

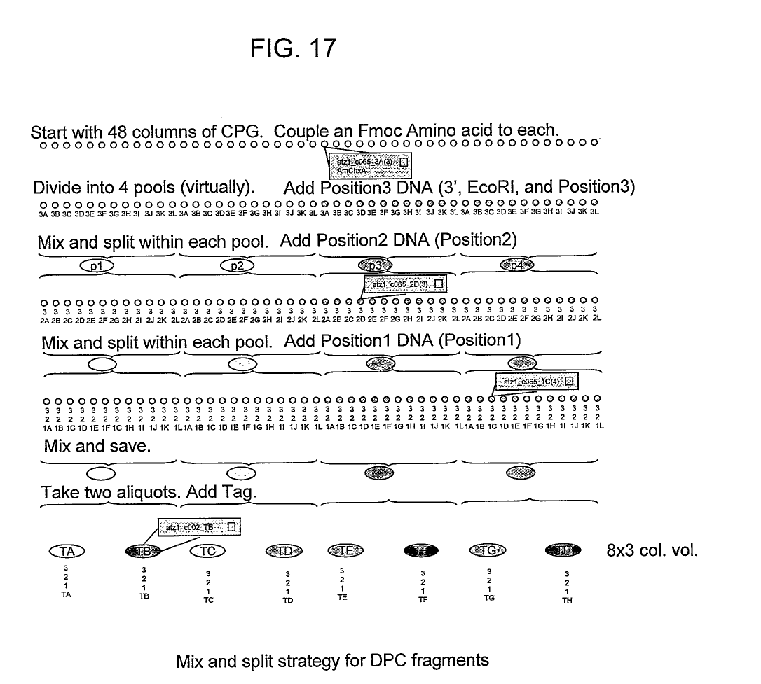

The mix and split strategy was used in preparing the oligos as shown in FIG. 17. The 3′-amino-modifier CPG derivatized with the appropriate Fmoc-protected amino acids were extended with the appropriate 3′-PCR primer sequence followed by the fragment specific codon to provide 48 distinct CPG products. These were then grouped into 4 groups of 12 representing the common Tag sequences shown in FIG. 15. Each of the 4 groups of 12 products were then mixed to provide 4 mixtures that were then split into 12 equal portions to provide 48 portions of CPG for further DNA synthesis. This same mix and split procedure was followed for codon 2 and codon 1. After the addition of the nucleotide sequences for codon 1 and the mix step, the 4 resulting mixtures were then split in half to yield 8 groups of CPG. These were then extended with the appropriate Position 0 codons followed by the 5′-PCR primer sequence. This provided the 8 unique Tag pools with 48 fragments in which Tags A&B contained 12 fragments, Tags C&D contained 12 fragments, etc. The codon sequences used in the mix and split synthesis are shown in Table 8.

TABLE 8
Codon sequences used in a mix and split synthesis
5′-PCR Primer-Position 0 Sequence

| | | | |
|---|---|---|---|
| atz1_c001_TA | CCACTACAACGCCAAACTC | (SEQ ID NO: 521) | 5′-PCR Primer-Pool A |
| atz1_c002_TB | CCACTACAACGAGCAACTC | (SEQ ID NO: 522) | 5′-PCR Primer-Pool B |
| atz1_c008_TC | CCACTACAACCAACCACTC | (SEQ ID NO: 523) | 5′-PCR Primer-Pool C |
| atz1_c011_TD | CCACTACAACTCAGCACTC | (SEQ ID NO: 524) | 5′-PCR Primer-Pool D |
| atz1_c019_TE | CCACTACAACCTAGGACTC | (SEQ ID NO: 525) | 5′-PCR Primer-Pool E |
| atz1_c032_TF | CCACTACAACATCCACCTC | (SEQ ID NO: 526) | 5′-PCR Primer-Pool F |
| atz1_c039_TG | CCACTACAACTCTACCCTC | (SEQ ID NO: 527) | 5′-PCR Primer-Pool G |
| atz1_c080_TH | CCACTACAACTCTCTGCTC | (SEQ ID NO: 528) | 5′-PCR Primer-Pool H |

Position 1 Sequence

| | | |
|---|---|---|
| atz1_c003_1A | ACCGTCAACAC | (SEQ ID NO: 529) |
| atz1_c005_1B | ACCACGAACAC | (SEQ ID NO: 530) |
| atz1_c006_1C | ACCCGTAACAC | (SEQ ID NO: 531) |
| atz1_c017_1D | ACAACCGACAC | (SEQ ID NO: 532) |
| atz1_c024_1E | ACGCACTACAC | (SEQ ID NO: 533) |
| atz1_c025_1F | ACCTCCTACAC | (SEQ ID NO: 534) |
| atz1_c027_1G | ACCCTGTACAC | (SEQ ID NO: 535) |
| atz1_c037_1H | ACGAAACCCAC | (SEQ ID NO: 536) |
| atz1_c038_1I | ACATGACCCAC | (SEQ ID NO: 537) |
| atz1_c041_1J | ACTTCTCCCAC | (SEQ ID NO: 538) |
| atz1_c012_1K | ACATCGCACAC | (SEQ ID NO: 539) |
| atz1_c034_1L | ACACTOACCAC | (SEQ ID NO: 540) |

Position 2 Sequence

| | | |
|---|---|---|
| atz1_c042_2A | TCCATTCCCTC | (SEQ ID NO: 541) |
| atz1_c044_2B | TCTACAGCCTC | (SEQ ID NO: 542) |
| atz1_c045_2C | TCCYFAGCCTC | (SEQ ID NO: 543) |
| atz1_cO51_2D | TCTAGCTCCTC | (SEQ ID NO: 544) |
| atz1_c052_2E | TCAGTCTCCTC | (SEQ ID NO: 545) |
| atz1_c054_2F | TCAACGTCCTC | (SEQ ID NO: 546) |
| atz1_c055_2G | TCCTGTTCCTC | (SEQ ID NO: 547) |
| atz1_c056_2H | TCGCTTTCCTC | (SEQ ID NO: 548) |
| atz1_c058_2I | TCCCTAAGCTC | (SEQ ID NO: 549) |
| atz1_c060_2J | TCTACCAGCTC | (SEQ ID NO: 550) |
| atz1_c064_2K | TCCTCTAGCTC | (SEQ ID NO: 551) |
| atz1_c031_2L | TCTCACACCTC | (SEQ ID NO: 552) |

Position 3 Sequence-3′-Primer Sequence

| | | |
|---|---|---|
| atz1_c065_3A | ACCTAACGCGAATTCCAC | (SEQ ID NO: 553) |
| atz1_c078_3B | ACCACATGCGAATTCCAC | (SEQ ID NO: 554) |
| atz1_cOSS_3C | ACCGCAATCGAATTCCAC | (SEQ ID NO: 555) |
| atz1_c086_3D | ACCTGCATCGAATTCCAC | (SEQ ID NO: 556) |
| atz1_c087_3E | ACGCTCATCGAATTCCAC | (SEQ ID NO: 557) |
| atz1_c088_3F | ACCCAGATCGAATTCCAC | (SEQ ID NO: 558) |
| atz1_c101_3G | ACTTCCGTCGAATTCCAC | (SEQ ID NO: 559) |
| atz1_c102_3H | ACCATCGTCGAATTCCAC | (SEQ ID NO: 560) |
| atz1_c108_3I | ACCGACTTCGAATTCCAC | (SEQ ID NO: 561) |
| atz1_c109_3J | ACGACCTTCGAATTCCAC | (SEQ ID NO: 562) |
| atz1_c112_3K | ACCCCTYFCGAAPrCCAC | (SEQ ID NO: 563) |
| atz1_c049_3L | ACCCAATCCGAATTCCAC | (SEQ ID NO: 564) |

The identity of the DPC fragments was confirmed by LC/MS analysis. Prior to the digestion protocol, any basic primary or secondary amines was acetylated to facilitate negative ionization. A solution of the DPC fragments in 0.3M TEAA (triethylammonium acetate) buffer, pH 7.2, was prepared (approx. 100 μmol in 200 μL), which was then treated with acetic anhydride (2 μL) for 30 min at room temperature. These samples were then evaporated to dryness. Oligo analytes were dissolved in 10 μL 10% methanol, and 1 μL internal standard solution (#1 below), 1 μL 10× buffer (#3 below), and 1 μL prepared Nuclease S1 aqueous Solution (#2 below) was added. Mixing was performed in a 600-ul plastic vial and the mixture was incubated at 37° C. in an air incubator for 2 hours.

The digestion control internal standard solution (#1) was comprised of 0.5 pmol/μl 1 μL A-phg-E stock solution (product m/z 709, 8.7 μM) and 1 μL (product m/z 896, 10 uM) stock solution mixed with 18 μL H₂O; Store this solution in −20° C. The 40 unit/μL enzyme solution (#2) was comprised of 1 μL commercial Nuclease S1 (Roche Diagnostics GMBH, 400 unit/ul) mixed with 9 μL H₂O. This solution is made right before using. The 10× digestion buffer (#3) was comprised of 330 mM sodium acetate, 500 mM naCl, 0.33 mM ZnSO₄, pH 4.5.

The digested samples were analyzed on an LC-MS system that consisted of a HPLC and Q-TOF premier mass spectrometer (Waters Corporation, Milford, Mass.). An Acquity column 100 mm×1 mm i.d. was installed and the samples were eluted using a gradient elution at 50 ul/min from 95% mobile phase A to 50% in 45 min. (HPLC mobile phase A: 1% hexafluoropropanol, 0.1% triethylamine in H₂O; mobile phase B: Methanol). Negative ions were analyzed with mass spectrometer.

The results of the LC/MS analysis of the DPC fragment examples are shown in Tables 9-12.

TABLE 9
Pool A&B Templates LC/MS Analysis

| Compound | Formula | Expected mass | Min M/Z | Max M/Z |
|---|---|---|---|---|
| Phosphate-3AMC7- | C17H36N5O7P | 452.2274 | 451.6394 | 452.6462 |
| ArgMe2-Ac | | | | |
| Phosphate-3AMC7- | C16H33N4O8P | 439.1958 | 437.9133 | 441.0056 |
| D-HoCit-Ac | | | | |
| Phosphate-3AMC7- | C16H32N3O8P | 424.1849 | 422.4642 | 426.8094 |
| LysFor-Ac | | | | |
| Phosphate-3AMC7- | C16H30N30O8P | 422.1692 | 421.1893 | 424.558 |
| Valeram-Ac | | | | |
| Phosphate-3AMC7- | C17H33N2O7P | 407.1947 | 405.8442 | 409.9454 |
| Cxhca-Ac | | | | |
| Phosphate-3AMC7- | C15H27N2O7P | 377.1478 | 375.2831 | 379.737 |
| Acyptene-Ac | | | | |
| Phosphate-3AMC7- | C13H27N2O7P | 353.1478 | 352.2337 | 354.8958 |
| Gaba-Ac | | | | |
| Phosphate-3AMC7- | C13H27N2O7P | 353.1478 | 352.1867 | 354.9552 |
| AMeProp-Ac | | | | |
| Phosphate-3AMC7- | C14H28N3O8P | 396.1536 | 395.8165 | 397.6751 |
| Gln-Ac | | | | |
| Phosphate-3AMC7- | C14H28N3O8P | 396.1536 | 395.8165 | 397.6751 |
| bGln-Ac | | | | |
| Phosphate-3AMC7- | C12H25N2O8P | 356.13 | Not Found | |
| Ser-Ac | | | | |
| Phosphate-3AMC7- | C12H25N2O8P | 356.13 | Not Found | |
| D-Ser-Ac | | | | |

TABLE 10
Pool C&D Templates LC/MS Analysis

| Compound | Formula | Expected mass | Min M/Z | Max M/Z |
|---|---|---|---|---|
| Phosphate-3AMC7- | C17H34N3O8P | 438.2005 | 437.8456 | 440.3896 |
| LysAc-Ac | | | | |
| Phosphate-3AMC7- | C21H35N4O8P | 501.2114 | 500.9587 | 503.4807 |
| Lys(Nic)-Ac | | | | |
| Phosphate-3AMC7- | C14H29N2O9PS | 431.1253 | 430.8351 | 433.2946 |
| Met(O2)-Ac | | | | |
| Phosphate-3AMC7- | C18H30N3O7P | 430.1743 | 429.8668 | 432.487 |
| A4PyrBA-Ac | | | | |
| Phosphate-3AMC7- | C18H30N3O7P | 430.1743 | 429.8668 | 432.487 |
| A3PyrBA-Ac | | | | |
| Phosphate-3AMC7- | C18H30N3O7P | 430.1743 | 429.8668 | 432.487 |
| SA4PBA-Ac | | | | |
| Phosphate-3AMC7- | C16H31N2O9PS | 457.141 | 456.8624 | 459.402 |
| THPO2Gly-Ac | | | | |
| Phosphate-3AMC7- | C16H31N2O7P | 393.1791 | 392.9446 | 395.4278 |
| ACHXA-Ac | | | | |
| Phosphate-3AMC7- | C17H28N3O7P | 416.1587 | 415.934 | 418.2854 |
| D3Pal-Ac | | | | |
| Phosphate-3AMC7- | C14H29N2O8P | 383.1583 | 383.0061 | 385.4017 |
| HoWSer(Me)-Ac | | | | |
| Phosphate-3AMC7- | C16H29N4O7P | 419.1696 | 418.9733 | 420.2447 |
| MeHis-Ac | | | | |
| Phosphate-3AMC7- | C11H23N2O7P | 325.1165 | 324.4351 | 327.3268 |
| Gly-Ac | | | | |

TABLE 11
Pool E & F Templates LC/MS Analysis

| Compound | Formula | Expected mass | Min M/Z | Max M/Z |
|---|---|---|---|---|
| Phosphate-3AMC7- | C14H29N2O7P | 367.1634 | 367.0273 | 369.3517 |
| Val-Ac | | | | |
| Phosphate-3AMC7- | C17H29N2O8P | 419.1583 | 418.9984 | 421.3014 |
| AFurBA-Ac | | | | |
| Phosphate-3AMC7- | C16H27N2O8P | 405.1427 | 404.9481 | 407.3366 |
| Ala2Fur-Ac | | | | |
| Phosphate-3AMC7- | C15H26N3O7PS | 422.1151 | 421.9503 | 424.2383 |
| Ala4Thz-Ac | | | | |
| Phosphate-3AMC7- | C18H35N2O7P | 421.2104 | 420.9165 | 423.3477 |
| AMChxA-Ac | | | | |
| Phosphate-3AMC7- | C17H29N2O7PS | 435.1355 | 434.6233 | 437.4323 |
| AThiBA-Ac | | | | |
| Phosphate-3AMC7- | C21H32N3O8P | 484.1849 | 483.6052 | 486.5442 |
| AZPC-Ac | | | | |
| Phosphate-3AMC7- | C20H30N3O7P | 454.1743 | 453.6125 | 456.4902 |
| CNHoPhe-Ac | | | | |
| Phosphate-3AMC7- | C15H29N2O7P | 379.1634 | 378.8961 | 381.3416 |
| CypAla-Ac | | | | |
| Phosphate-3AMC7- | C15H26N3O7PWS | 422.1151 | 421.8904 | 424.311 |
| Dala4Thz-Ac | | | | |
| Phosphate-3AMC7- | C20H33N2O9P | 475.1845 | 474.7141 | 477.539 |
| DiMeoPhe-Ac | | | | |
| Phosphate-3AMC7- | C17H28N3O7P | 416.1587 | 415.8744 | 418.4505 |
| L3Pal-Ac | | | | |

TABLE 12
Pool G & H Templates LC/MS Analysis

| Compound | Formula | Expected mass | Min M/Z | Max M/Z |
|---|---|---|---|---|
| Phosphate-3AMC7- | C18H35N2O7P | 421.2104 | 421.0835 | 423.4081 |
| Cha-Ac | | | | |
| Phosphate-3AMC7- | C21H31N2O7PS | 485.1511 | 484.1497 | 488.0578 |
| ABztB-Ac | | | | |
| Phosphate-3AMC7- | C16H27N2O7PS | 421.1198 | 420.986 | 423.2295 |
| Thi-Ac | | | | |
| Phosphate-3AMC7- | C24H33N2O7P | 491.1947 | 490.4798 | 495.0777 |
| DBip-Ac | | | | |
| Phosphate-3AMC7- | C19H29F2N2O7P | 465.1602 | 464.7077 | 467.5954 |
| F2HoPhe-Ac | | | | |
| Phosphate-3AMC7- | C19H36N3O8P | 464.2162 | 463.9187 | 464.6658 |
| Freidam-Ac | | | | |
| Phosphate-3AMC7- | C22H33N4O7P | 495.2009 | 493.9567 | 497.9117 |
| His(Bn)-Ac | | | | |
| Phosphate-3AMC7- | C20H31N2O7P | 441.1791 | 440.5078 | 444.1657 |
| Indanygly-Ac | | | | |
| Phosphate-3AMC7- | C20H31N2O7P | 441.1791 | 440.4843 | 444.1423 |
| Styrylala-Ac | | | | |
| Phosphate-3AMC7- | C24H33N2O7P | 491.1947 | 490.6873 | 495.0122 |
| LBip-Ac | | | | |
| Phosphate-3AMC7- | C15H31N2O7P | 381.1792 | 380.5196 | 383.8154 |
| Leu-Ac | | | | |
| Phosphate-3AMC7- | C17H27N2O7P | 401.1478 | 399.6447 | 404.5285 |
| Phg-Ac | | | | |

Example of DPC Assembly of Fragments. Pools A, E & H were shown to have very weak affinity for the target kinases, Abl & KDR. FOPP Target Binding Element was shown to have weak affinity for Abl and good affinity for KDR. DPC was used to assemble libraries that combined the FOPP Target Binding Element with the weak affinity DPC fragments exemplified above. Structures of compounds in Pools A, E & H are shown in Tables 13-15.

TABLE 13
Structures of Compounds in Pool A

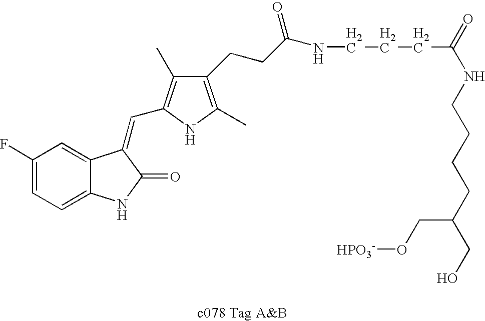

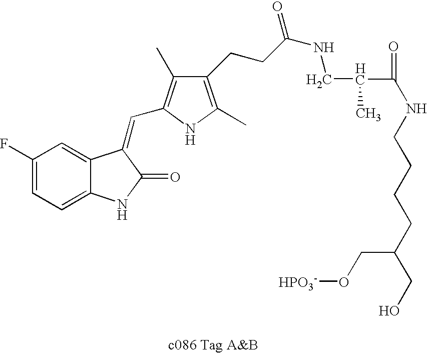

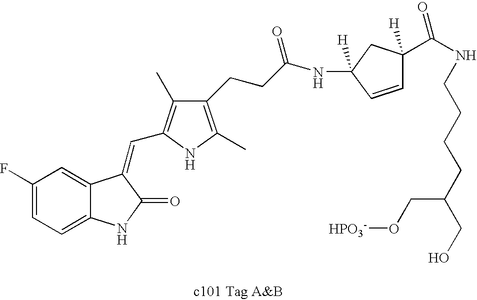

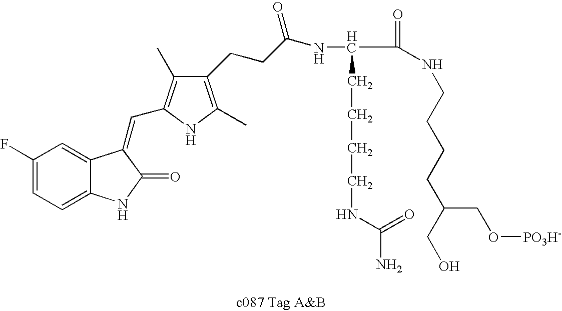

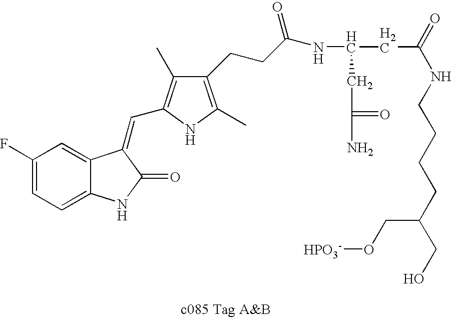

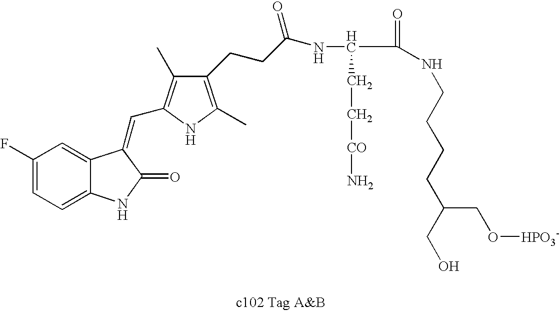

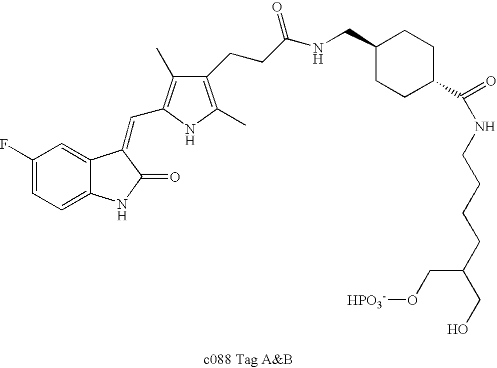

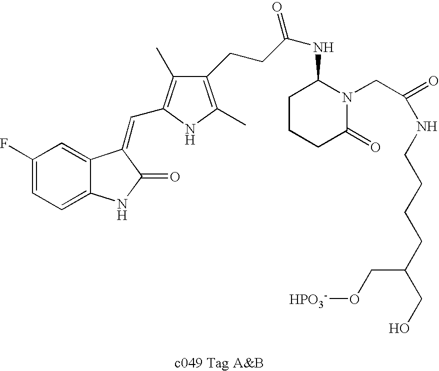

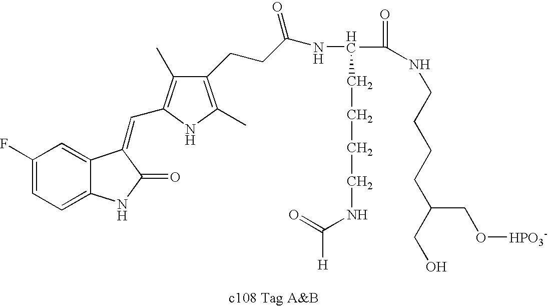

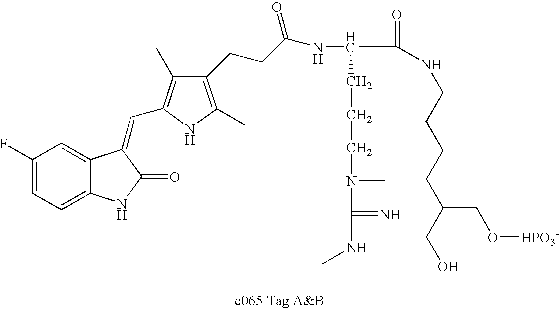

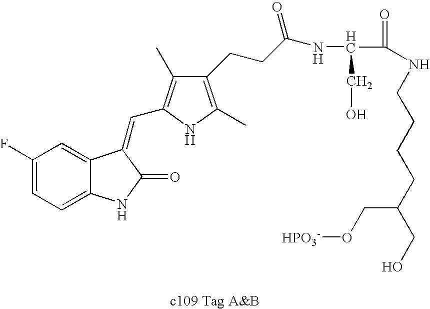

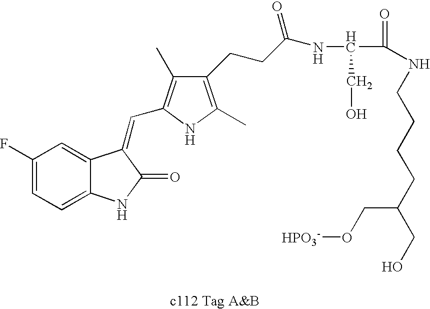

TABLE 14
Structures of Compounds in Pool E

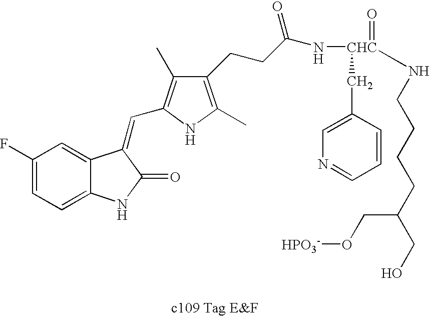

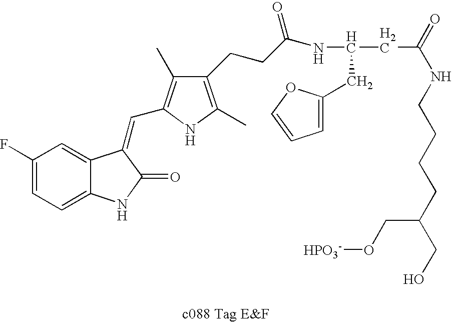

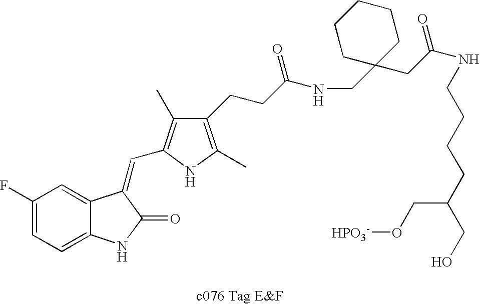

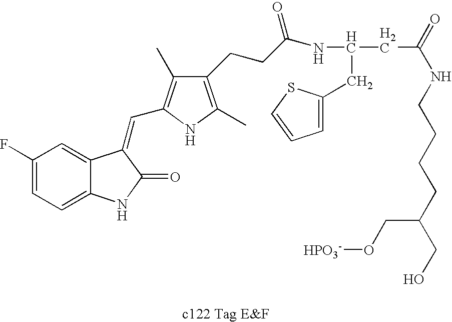

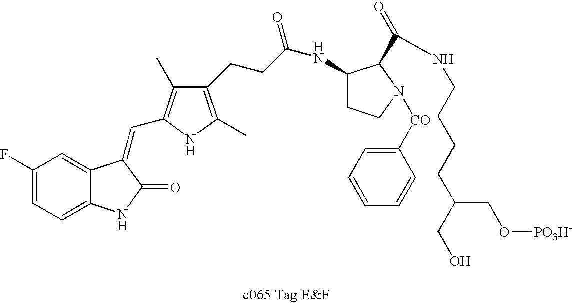

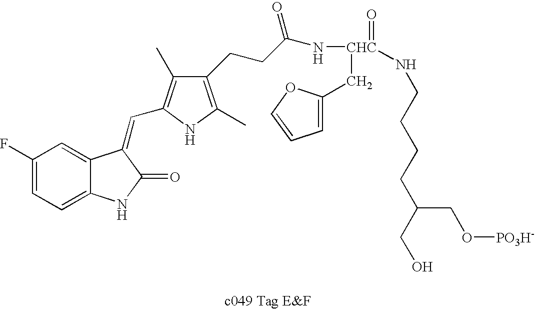

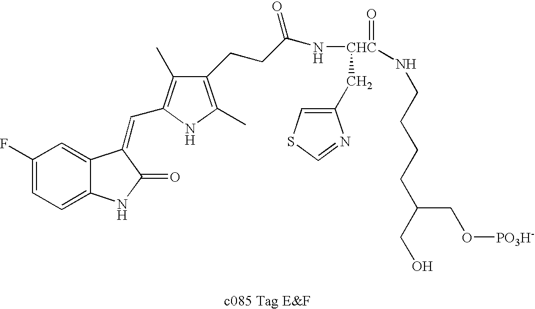

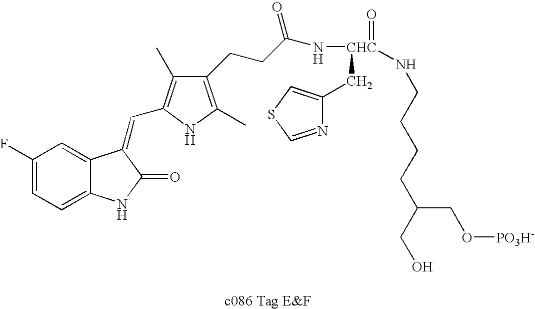

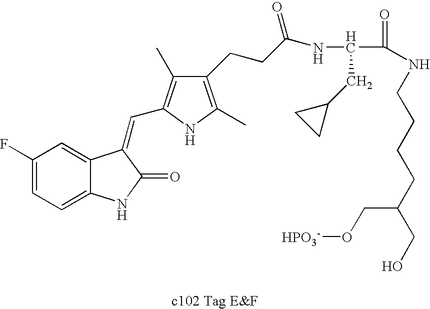

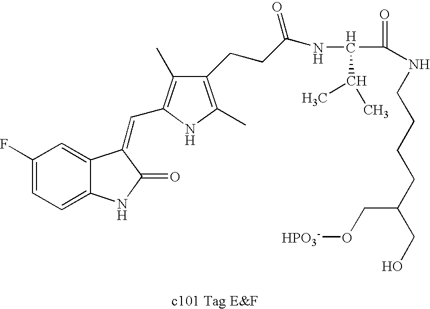

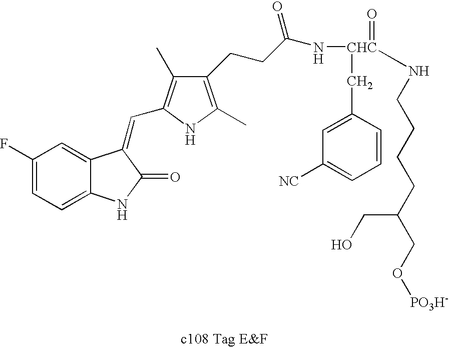

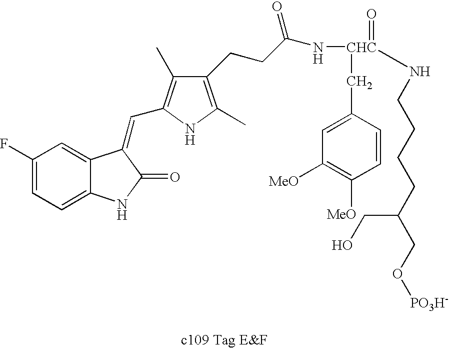

TABLE 15
Structures of Compounds in Pool H

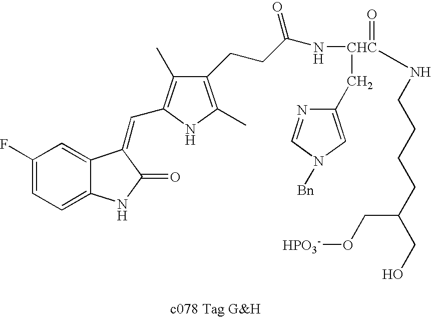

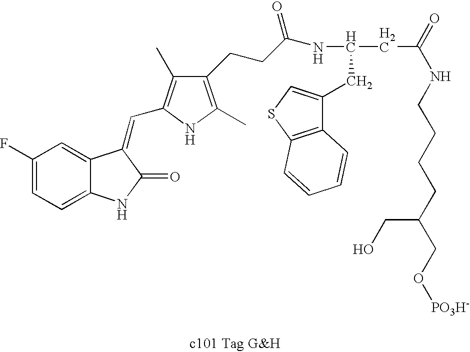

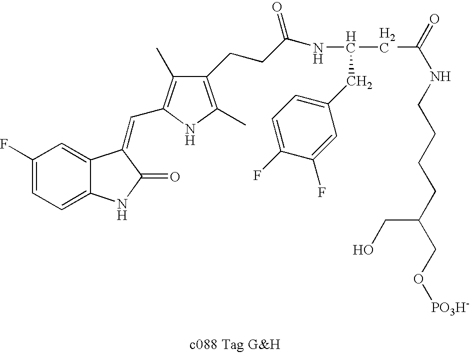

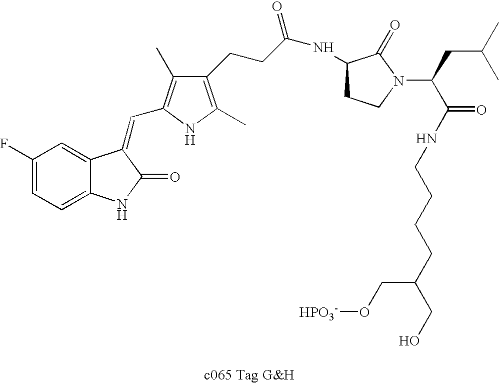

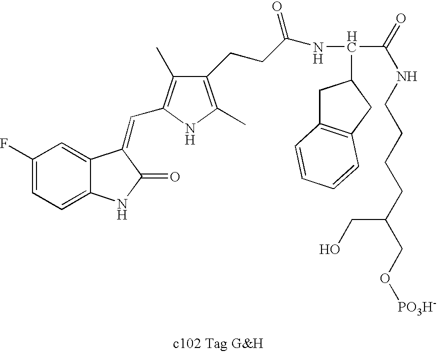

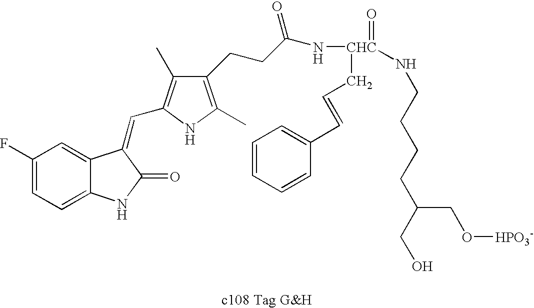

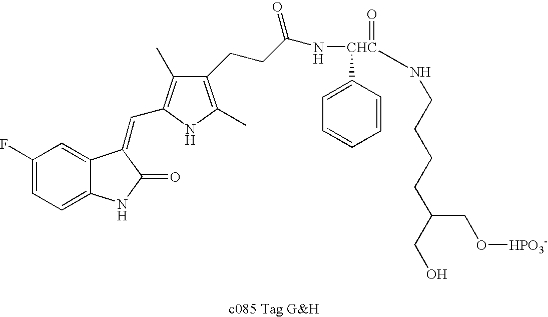

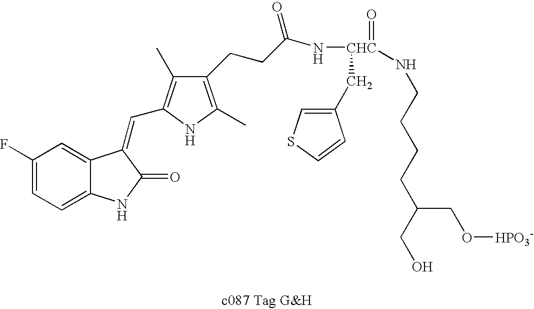

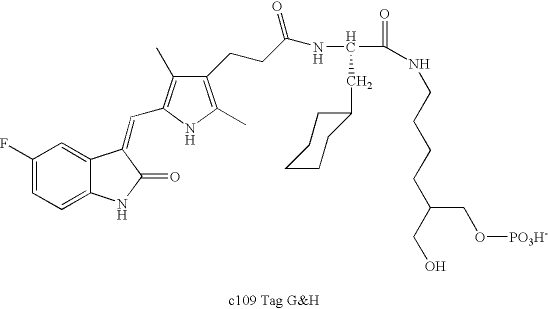

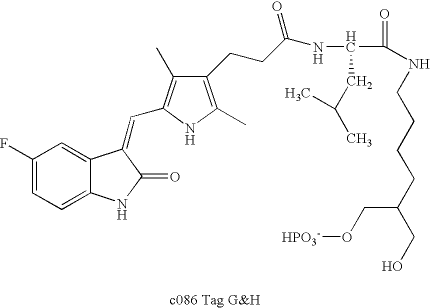

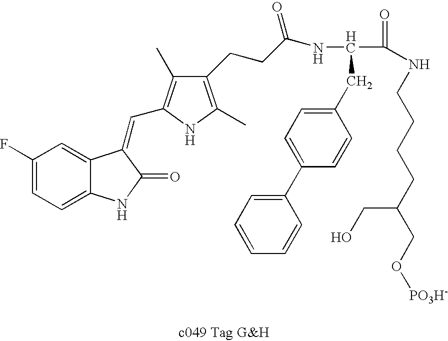

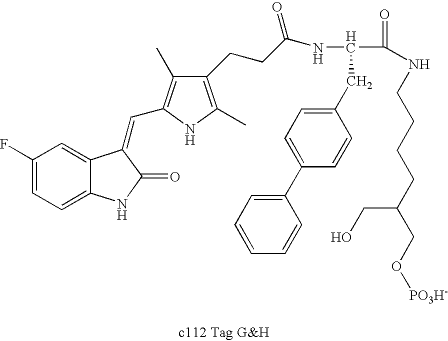

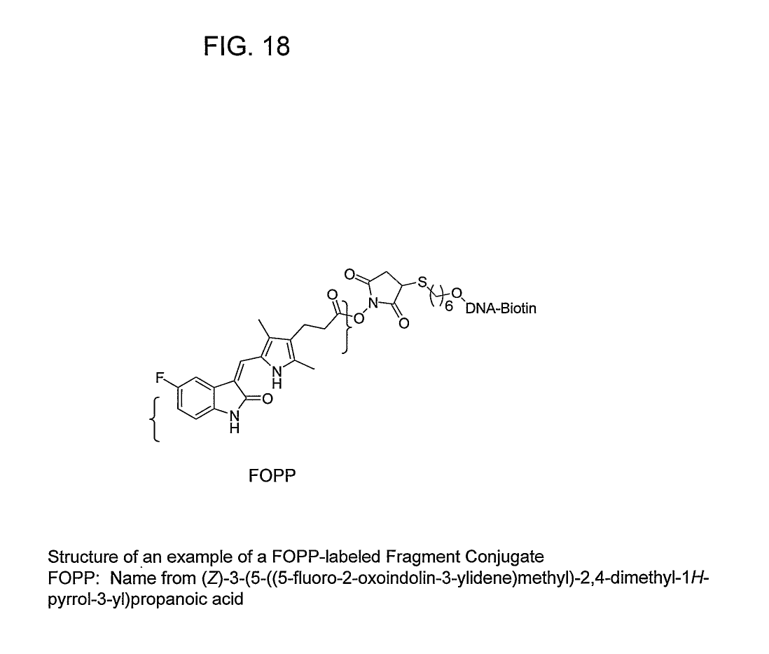

Preparation of the DNA-FOPP Target Binding Element Strand for DPC Assembly of fragments. A general protocol for preparing DNA-OSu-R reagent can be found, for example, in “Ordered Multistep Synthesis in a Single Solution Directed by DNA Templates” Snyder, T. M. and Liu, D. R. Angew. Chem. Int. Ed. 44, 7379-7382 (2005). Briefly, a 10-mer DNA was prepared: 5′-trityl-S-GTG GAA TTC G-3′-biotin. The deprotection of the trityl group proceeded as follows. First, 40 μL of DNA (50-100 μM) was mixed with 2 μL 2.0 M TEAA (pH=7.0) and 6 μL AgNO₃ (1 M in H₂O) was added. The reaction was kept at RT on a vortexer for 30 min before 8.4 μL DTT (1 M solution in H₂O) was added and vortexed for 5 min to precipitate excess DTT. The yellowish suspension was loaded to a NAP 5 column and the 1 mL collected DNA solution was reacted with N-hydroxymaleimide in the next step. Next, 10 mg N-hydroxymaleimide was added with 125 μL H₂O and 125 μL MOPS (1M, pH=7.5). The solution turned brown immediately upon the addition of MOPS, and it was quickly mixed with the DNA solution obtained from last step. The reaction mixture was kept under RT for 30 min, then was placed in a speedvac to reduce the volume under 1 mL before being desalted on a NAP10 column. The product was purified by HPLC and then reacted with FOPP Target Binding element carboxylic acid. Then, 2.04 μmol FOPP—COOH was dissolved in 50 μL DMF, 0.5 mg EDC was dissolved in 50 μL DMF; and then 20 μL EDC solution, 25 μL FOPP—COOH solution and 5 μL DMF were combined. The reaction mixture was kept under RT for 20 min before being added to the DNA-solution (16 μL MES (0.5M, pH=6.5), 24 μL H₂O, and 40 μL DNA prepared in step 3.). The reaction was maintained at RT for 5-10 min, then desalted by a NAP5 column and purified by HPLC. After collecting the product fraction from the HPLC, 1:5 (v:v) 6% TFA was added directly into the fraction before putting it on the lyophilizer. The final product dried from TFA-containing lyophilization was yellow and in a semi-dry form, and was stored at −80° C. in this form. Prior to use, the product was brought to 10-20 μM with H₂O, the concentration was measured, and then it was immediately used in DPC reaction. The structure of the FOPP-labeled DPC Fragment was confirmed by LC/MS (expected mass (6-): 710.6466; Observed mass (6-): 710.6719), as shown in FIG. 18.

DPC-Based Fragment Assembly. Assembly was performed under the following conditions: 1 M NaCl, 0.2 M MES (pH=6.5), 1 μM template, 2 μM DNA-FOPP reagent, at room temperature for 1 hour. The reaction was quenched with 1:20 (volume:volume) Tris-HCl buffer (1 M, pH=7.2), then subjected to a streptavidin tip purification to remove biotinylated reagents. The solution then was collected and dried in a speedvac until the volume was less than 0.5 mL to be desalted on a NAP5 column. The 1 mL solution collected from NAP5 column was lyophilized and analyzed by LC-MS. The expected and observed molecular ions are shown in Tables 16-18.

TABLE 16
DPC Assembled Fragments LC/MS Analysis (Pool A)

| | | | Expected | | |
|---|---|---|---|---|---|
| Compound | Formula | Codon | mass | Min. M/Z | Max. M/Z |
| Phosphate-3AMC7- | C29H40FN4O8P | c078 | 621.249 | 620.3623 | 621.7786 |
| Gaba_FOPP | | | | | |
| Phosphate-3AMC7- | C29H40FN4O8P | c086 | 621.249 | 620.2646 | 621.7053 |
| AMeProp_FOPP | | | | | |
| Phosphate-3AMC7- | C31H40FN4O8P | c101 | 645.249 | 644.597 | 648.6808 |
| Acyptene_FOPP | | | | | |
| Phosphate-3AMC7- | C32H46FN6O9P | c087 | 707.297 | 706.6595 | 710.0528 |
| D-HoCit_FOPP | | | | | |
| Phosphate-3AMC7- | C30H41FN5O9P | c085 | 664.2548 | 663.237 | 666.9654 |
| bGln_FOPP | | | | | |
| Phosphate-3AMC7- | C30H41FN5O9P | c102 | 664.2548 | 663.0556 | 668.3527 |
| Gln_FOPP | | | | | |
| Phosphate-3AMC7- | C33H46FN4O8P | c088 | 675.2959 | 674.8134 | 677.8547 |
| Cxcha_FOPP | | | | | |
| Phosphate-3AMC7- | C32H43FN5O9P | c049 | 690.2704 | 689.3646 | 692.9398 |
| Valeram_FOPP | | | | | |
| Phosphate-3AMC7- | C32H45FN5O9P | c108 | 692.2861 | 691.5269 | 695.3058 |
| LysFor_FOPP | | | | | |
| Phosphate-3AMC7- | C33H49FN7O8P | c065 | 720.3286 | 719.7717 | 722.909 |
| ArgMe2_FOPP | | | | | |
| Phosphate-3AMC7- | C28H38FN4O9P | c112 | 623.2282 | 623.04 | 625.559 |
| Ser_FOPP | | | | | |
| Phosphate-3AMC7- | C28H38FN4O9P | c109 | 623.2282 | 623.0205 | 625.5002 |
| D-Ser_FOPPanchor | | | | | |

TABLE 17
DPC Assembled Fragments LC/MS Analysis (Pool E)

| | | | Expected | | |
|---|---|---|---|---|---|
| Compound | Formula | Codon | mass | Min. M/Z | Max. M/Z |
| Phosphate-3AMC7- | C33FH41N5O8P | c109 | 684.2599 | 684.0737 | 686.4737 |
| 3Pal_FOPP | | | | | |
| Phosphate-3AMC7- | C33FH42N4O9P | c088 | 687.2595 | 687.0734 | 689.4734 |
| AFurBA_FOPP | | | | | |
| Phosphate-3AMC7- | C34FH48N4O8P | c078 | 689.3116 | 689.1254 | 691.5254 |
| AMChxA_FOPP | | | | | |
| Phosphate-3AMC7- | C33FH42N4O8PS | c112 | 703.2367 | 703.0506 | 705.4506 |
| AThiBA_FOPP | | | | | |
| Phosphate-3AMC7- | C37FH45N5O9P | c065 | 752.2861 | 752.0999 | 754.4999 |
| AZPC_FOPP | | | | | |
| Phosphate-3AMC7- | C32FH40N4O9P | c049 | 673.2439 | 673.0577 | 675.4577 |
| Ala2Fur_FOPP | | | | | |
| Phosphate-3AMC7- | C31FH39N5O8PS | c085 | 690.2163 | 690.0302 | 692.4302 |
| Ala4Thz_FOPP | | | | | |
| Phosphate-3AMC7- | C31FH42N4O8P | c102 | 647.2646 | 647.0785 | 649.4785 |
| CypAla_FOPP | | | | | |
| Phosphate-3AMC7- | C31FH39N5O8PS | c086 | 690.2163 | 690.0302 | 692.4302 |
| D_Ala4Thz_FOPP | | | | | |
| Phosphate-3AMC7- | C36FH46N4O10P | c109 | 743.2857 | 743.0996 | 745.4996 |
| Phe(MeO2)_FOPP | | | | | |
| Phosphate-3AMC7- | C30FH42N4O8P | c101 | 635.2646 | 635.0785 | 637.4785 |
| Val_FOPP | | | | | |
| Phosphate-3AMC7- | C36FH43N5O8P | c108 | 722.2755 | 722.0894 | 724.4894 |
| CNHoPhe_FOPP | | | | | |

TABLE 18
DPC Assembled Fragments LC/MS Analysis (Pool H)

| | | | Expected | | |
|---|---|---|---|---|---|
| Compound | Formula | Codon | mass | Min. M/Z | Max. M/Z |
| Phosphate-3AMC7- | C38H46FN6O8P | c078 | 763.3021 | 762.6799 | 765.6587 |
| His(Bn)_FOPP | | | | | |
| Phosphate-3AMC7- | C37H44FN4O8PS | c101 | 753.2523 | 752.5948 | 755.9703 |
| ABztB_FOPP | | | | | |
| Phosphate-3AMC7- | C35H42F3N4O8P | c088 | 733.2614 | 732.6077 | 736.3311 |
| F2HoPhe_FOPP | | | | | |
| Phosphate-3AMC7- | C35H49FN5O9P | c065 | 732.3174 | 731.4594 | 735.0368 |
| Freidam_FOPP | | | | | |
| Phosphate-3AMC7- | C36H44FN4O8P | c102 | 709.2803 | 708.0261 | 712.7701 |
| Indanylgly_FOPP | | | | | |
| Phosphate-3AMC7- | C36H44FN4O8P | c108 | 709.2803 | 708.5441 | 712.3941 |
| Styrylala_FOPP | | | | | |
| Phosphate-3AMC7- | C33H40FN4O8P | c085 | 669.249 | 668.5738 | 671.8665 |
| Phg_FOPP | | | | | |
| Phosphate-3AMC7- | C32H40FN4O8PS | c087 | 689.221 | 688.5423 | 692.1912 |
| Thi_FOPP | | | | | |
| Phosphate-3AMC7- | C34H48FN4O8P | c109 | 689.3116 | 688.6494 | 692.0565 |
| Cha_FOPP | | | | | |
| Phosphate-3AMC7- | C31H44FN4O8P | c086 | 649.2803 | 648.3741 | 652.2355 |
| Leu_FOPP | | | | | |
| Phosphate-3AMC7- | C40H46FN4O8P | c049 | 759.2959 | 758.7568 | 762.3244 |
| DBip_FOPP | | | | | |
| Phosphate-3AMC7- | C40H46FN4O8P | c112 | 759.2959 | 758.4529 | 762.4684 |
| LBip_FOPP | | | | | |

### Example 4

### Selection of Anchor-Based DPC Libraries

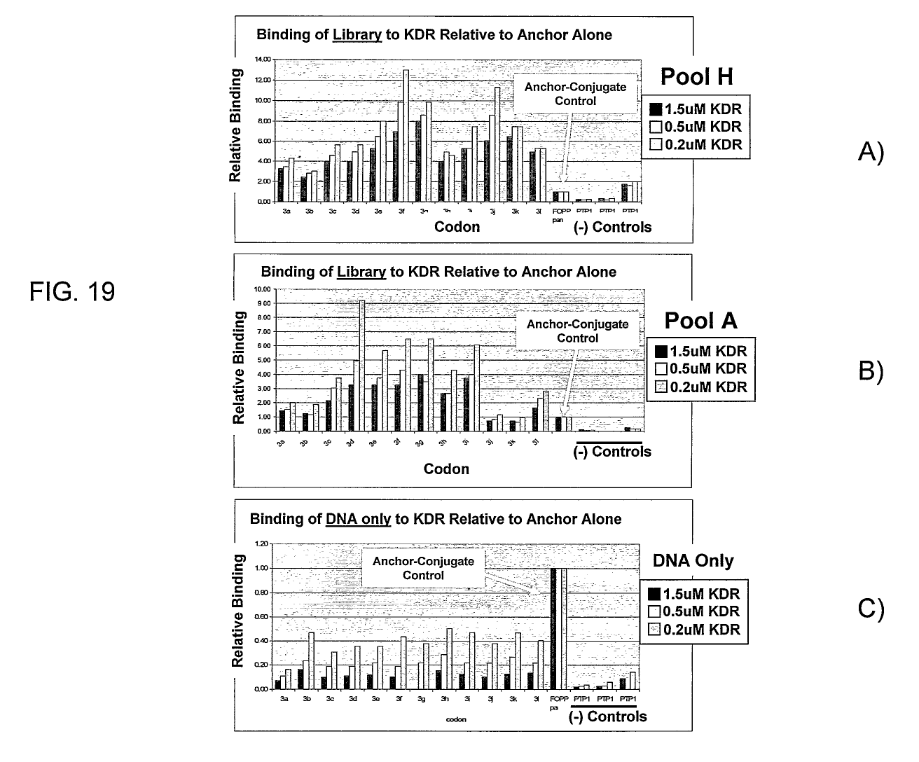

The ability of amino acid-based fragments to enhance the binding of an anchor when the two are conjugated to one another has been demonstrated. FIG. 19 shows an example of binding of two 12-member anchor-based libraries to KDR. Two 12-member libraries containing the DNA conjugate of the anchor FOPP linked to a single diversity position, Pools H and A (see structures in Tables 13 and 15), were selected against the kinase protein target KDR.

Each member contains FOPP (see FIG. 18) as an anchor and a single amino acid as the conjugated variable fragment, Each individual member of each library is designated by codons 3a-3l. The relative binding of each member compared to the anchor control was determined at three different KDR concentrations as described in Methods (below). The binding of the corresponding linked DNA conjugate void of the anchor and the amino acid comprising the single point of diversity for each member was also determined (See text for discussion).

### Methods

Indicated amounts of N-terminally 6×-His-tagged cytoplasmic domain from aa790-end (aa1357) of KDR (Upstate) was immobilized to Qiagen Ni²⁺/nitrilotriacetic acid Superflow™ resin in 50 mM Tris, pH7.5, 300 mM NaCl, 270 mM sucrose, 0.03% Brij-35 for 2 hours at 4° C. Using the same buffer, resin was washed three times and resuspended as a 25% slurry. 10 μL resin beds of KDR resins were pelleted and washed twice with 100 μL of binding buffer (25 mM Tris, pH 7.5/10 mM MgCl₂/1 mM Tris(2-carboxyethyl) phosphine/150 mM NaCl) and supernatants removed.

For binding experiments, 10 μL of the following mix was added to each resin: 12 nM 12-membered FOPP anchored libraries, 1 nM FOPP-DNA conjugate parent, 1 nM each of PTP1B inhibitors (see figure) conjugated to DNA, 5 μM decoy DNA (sequence=5′CACTACAACACATCCCTCACCGTCAACACTCCATTCCCTCAC 3′ (SEQ ID NO: 565), 25 mM Tris, pH7.5, 10 mM MgCl₂, 1 mM Tris(2-carboxyethyl) phosphine, 150 mM NaCl. For binding/competition experiments, 20 μL of the same mix at half the library and control concentrations in the presence of inhibitor and 0.5% DMSO was used. Libraries and resins were incubated at room temperature for one hour with slight agitation on a vortexer. 150 μL of binding buffer was added to each sample, resin resuspended and transferred separately to Ultrafree-MC 5 μm spin filter units (Millipore) and centrifuged briefly to remove buffer. Resin was washed with 2×200 μL of binding buffer and recentrifuged. Resins were then resuspended in 100 μL binding buffer and transferred to 0.2 mL thin-walled PCR tube. Resins were centrifuged, supernatants removed and resins resuspended in 50 μL of 6 M guanidine-HCl. Resins were heated at 70° C. for twenty minutes, centrifuged, and supernatants transferred to 500 μL of PN buffer from Qiagen nucleotide removal kit. Samples were desalted according to manufacturer's protocol and eluted with 100 μL water.

Quantitative real-time PCR was used to quantitate small molecule-DNA conjugates in the applied material and the selected eluates. Briefly, the libraries and controls contain library-specific DNA sequences that can be used as a common 5′ priming spot for each member of the library. The 3′ primer is specific for the codons used to generate the DPC libraries. Biorad SYBRIQ was used to prepare mixes containing 0.5 μM 5′ library-specific primer and 0.5 μM 3′ codon-specific primers (one PCR reaction specific for each 3′ codon). Five μL ( 1/20^{th}) of each sample was added to the PCR reaction mixes specific for each codon and quanitative real-time PCR was performed on a Biorad ICycler. As a pre-binding control, the library mixes applied to the resins were diluted 1/100 and five μL of each were added to PCR mixes. Percent binding for each codon was determined by the relationship below and normalized to the anchor conjugate control.

$$[ \frac{the amount of PCR product in the binding sample}{\begin{matrix}the amount of PCR product in \\ the pre - binding sample\end{matrix}} ] \times 100$$

Relative to the anchor alone, the majority of conjugates in both libraries (Pools H and A) bound significantly more tightly. For example, in Pool H the conjugate containing the amino acid-based fragment, coded by codon 3f, bound approximately 10-fold more tightly than the anchor alone control. Likewise, for Pool A the conjugate containing the amino acid-based fragment coded by codon 3d bound 10-fold more tightly than the anchor alone control. As expected for the stringency of the selection process, the differential binding was most evident at the lower concentrations of the target protein. In control studies, the corresponding DNA-alone controls that did not contain any fragments or an anchor that could serve as a target binding element did not show significant binding to KDR relative to the anchor alone (note change in scale). These studies demonstrate the ability of fragments to enhance the binding of a known anchor.

### Example 5

### Discovery of Novel Ligands to Other Targets

Procedures of Example 4 may be applied to other targets of interest such as phosphatases, proteases, receptors, ion channels, oxidases and reductases, catabolic and anabolic enzymes, pumps, and electron transport proteins. Examples of targets include BCR/Abl, BACE, HCV protease, P2Y(12), PTP1b, Renin, TNF-α and PAI-1.

The library of fragments may be selected against other targets such as BCR/Abl, using PCR to amplify sequences of binders. In one approach to the actual protein binding selections, DPC-fragment libraries are dissolved in aqueous binding buffer in one pot and equilibrated in the presence of immobilized target protein. Non-binders are washed away with buffer. Those molecules that may be binding through their attached DNA templates rather than through their fragment moieties are eliminated by washing the bound library with unfunctionalized DNA templates lacking PCR primer binding sites. Remaining ligands bound to the immobilized target are eluted.

To increase enrichment, one may iterate a selection by loading eluant from a first selection into a second selection to multiply the net enrichment. No intervening amplification of template is required. Iterating library selections can lead to very large enrichments of desired molecules. In certain embodiments, a first round of selection provides at least a 50-fold increase in the number of binding ligands. Preferably, the increase in enrichments is over 100-fold, more preferably over 1,000 fold, and even more preferably over 100,000-fold. Subsequent rounds of selection may further increase the enrichment 100-fold over the original library, preferably 1,000-fold, more preferably over 100,000-fold, and most preferably over 1,000,000-fold.

In vitro selections can also select for specificity in addition to binding affinity. Library screening methods for binding specificity typically require duplicating the entire screen for each target or non-target of interest.

In contrast, selections for specificity can be performed in a single experiment by selecting for target binding as well as for the inability to bind one or more non-targets. Thus, the library can be pre-depleted by removing library members that bind to a non-target. Alternatively, or in addition, selection for binding to the target molecule can be performed in the presence of an excess of one or more non-targets. To maximize specificity, the non-target can be a homologous molecule. If the target molecule is a protein, appropriate non-target proteins include, for example, a generally promiscuous protein such as an albumin. If the binding assay is designed to target only a specific portion of a target molecule, the non-target can be a variation on the molecule in which that portion has been changed or removed. See, e.g., U.S. Patent Application Publication No. 2004/0180412 A1 by Liu et al.

The DNA templates that encode and direct the syntheses of the target binding molecules may be amplified by any suitable technique, e.g., by PCR; nucleic acid sequence-based amplification (see, e.g., Compton, 1991, Nature, 350: 91-92), amplified anti-sense RNA (see, e.g., van Gelder et al., 1988, Proc. Natl. Acad. Sci. USA 85: 77652-77656); self-sustained sequence replication systems (Gnatelli et al., 1990, Proc. Natl. Acad. Sci. USA 87: 18741878); polymerase-independent amplification (see, e.g., Schmidt et al., 1997, Nucleic Acids Res. 25: 4797-4802, and in vivo amplification of plasmids carrying cloned DNA fragments. Description of PCR methods are found, for example, in Saiki et al., 1985, Science 230: 1350-1354; Scharf et al., 1986, Science 233: 1076-1078; and in U.S. Pat. No. 4,683,202. Ligase-mediated amplification methods such as Ligase Chain Reaction (LCR) may also be used. In general, any means allowing faithful, efficient amplification of selected nucleic acid sequences can be employed in the method of the present invention. It is preferable, although not necessary, that the proportionate representations of the sequences after amplification reflect the relative proportions of the sequences in the mixture before amplification.

Purification completes one cycle of translation, selection and amplification, yielding an enriched sub-population of DNA-fragments having binding affinities to the target protein.

The above process can be repeated until a subset of DPC-fragments are identified that bind to the target with desired affinity ranges, for example, “moderate affinity” (1 μM<K_{D}<10 μM), “moderately high affinity” (100 nM<K_{D}<1 μM), or “high affinity” (K_{D}<100 nM, e.g., K_{D}<50 nM or 20 nM, or “very high affinity” (1 nM or sub-nanomolar<K_{D}<10 nM)). Additionally, deconvolution is performed on the set of binders from the mixture to obtain SAR of the target binding elements themselves. This allows one to infer where on a fragment substitution or other modifications may or may not be tolerated. Additionally, information can be obtained on SAR relating to the specific functionalities that should be tolerated in the subsequent DPC generated libraries for attaching fragments to each other or to other scaffolds.

To investigate a particular target binding element, the DNA sequence associated with the molecule can be sequenced using conventional approaches, which sequence can then be used to deconvolute the identity (e.g., structure and synthetic history) of the target binding element.

Sequencing can be performed by a standard dideoxy chain termination method, or by chemical sequencing, e.g., using the Maxam-Gilbert sequencing procedure. Alternatively, the sequence can be determined by hybridization to a chip. For example, a single-stranded DNA associated with a detectable moiety such as a fluorescent moiety is exposed to a chip bearing a large number of clonal populations of single-stranded nucleic acid analogs of known sequences, each clonal population being present at a particular addressable location on the chip. The unknown sequences are permitted to anneal to the chip sequences. The position of the detectable moieties on the chip then is determined. Based on the location of the detectable moiety and the immobilized sequence at that location, the sequence of the template can be determined. It is contemplated that large numbers of such oligonucleotides can be immobilized in an array on a chip or other solid support.

A combinatorial library can be prepared by a DPC process in which the identified target binding elements in the form of building blocks are incorporated. The target binding elements can be linked directly, via linking moieties or via scaffolds. The chemical assembly of the target binding elements using DPC to generate a library can be accomplished using chemical methodologies that have been established as amenable to DPC using strategies that have been shown appropriate for the multistep assembly of combinatorial libraries, as discussed above. This DPC-generated library is then selected against the target to identify those target binding elements that yield a more elaborated molecule with increased affinity for the target. See, e.g., U.S. Patent Application Publication No. 2004/0014090 A1 by Neri et al. and PCT International Publication No. WO 03/076943 A1; Gartner et al. Science, vol. 305, pp1601-1605, 2004; Doyon, et al., JACS, vol. 125, pp 12372-12373, 2003.

The relative abundance of codons present in the library recovered from the selection is compared against the relative abundance of codons in the library prior to the selection. If a particular TBE, functionality, or scaffold, binds preferentially to the target, the relative abundance of the codons for the entity will increase as a result of the selection. If a particular entity is disfavored in binding, its relative frequency will decrease as a result of the selection. Additionally, optimal combinations of TBE's or functionalities, regardless of the scaffold in which they find themselves, may be preferred by the target binding site, and these interactions will be reflected in positive co-variance of pairs of codon frequencies. These data can be tabulated and analyzed to determine the optimal set of TBE's/codons to carry into a second or next round of selection.

The above example is envisioned to be applicable in general to other kinases, e.g., tyrosine kinases. Other exemplary kinases of therapeutic interest: VEGFR, PDGFR, EGFR, c-Kit, Flt-3, Src, Lck, Aurora, CDK's, JAK, IKK, p38, Raf, ERB B1&2, and JNK.

### INCORPORATION BY REFERENCE

The entire disclosure of each of the publications and patent documents referred to herein is incorporated by reference in its entirety for all purposes to the same extent as if each individual publication or patent document were so individually denoted.

### EQUIVALENTS

The invention may be embodied in other specific forms without departing form the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method for identifying a target binding element capable of binding to a binding domain disposed within a binding site of a target molecule, wherein the target binding element has a K_{D} of 10 mM or lower, the method comprising:
(a) combining a target molecule with a plurality of pre-selected test molecules under conditions that permit a test molecule to bind to a binding domain of the target molecule, wherein each test molecule comprises a target binding element associated with a corresponding oligonucleotide having a nucleotide sequence that (i) identifies the target binding element, (ii) contains an amplification sequence, and (iii) is substantially incapable of hybridizing to the nucleotide sequences associated with other target binding elements;
(b) harvesting a target binding element that binds to the target molecule with a K_{D} of 10 mM or lower; and
(c) determining the sequence of the oligonucleotide associated with the target binding element harvested in step (b) so as to identify the target binding element having a K_{D} of 10 mM or lower with the binding site.

2-3. (canceled)

4. The method of claim 1, further comprising the step of, before step (b), washing away target binding elements that bind to the target with K_{D} greater than 1 M.

5. The method of claim 1, wherein the target binding element has a mass ranging from 90 to 500 daltons.

6. The method of claim 5, wherein the target binding element has a mass ranging from 150 to 350 daltons.

7. The method of claim 1, wherein the target binding element has a K_{D} with the target molecule selected from the group consisting of less than 1 nM, from 1 nM to 100 nM, from 100 nM to 10 μM, from 10 μM to 100 μM, and from 100 μM to 10 mM.

8-30. (canceled)

31. A composition comprising a plurality of test molecules, wherein each of substantially all of the test molecules comprises a target binding element associated with a corresponding oligonucleotide having a nucleotide sequence that (i) identifies the target binding element, (ii) contains an amplification sequence, and (iii) is substantially incapable of hybridizing to the nucleotide sequences associated with other target binding elements.

32. (canceled)

33. The composition of claim 31, wherein substantially all of the target binding elements has a K_{D} with a binding site greater than 10 μM.

34. The composition of claim 31, wherein substantially all of the target binding elements has a molecular weight less than 400 daltons.

35. The composition of claim 31, wherein substantially all of the target binding elements are attached to the oligonucleotide via one or more functional groups associated with the target binding elements.

36. The composition of claim 35, wherein the functional group is selected from the group consisting of amines, carboxylic acids, acid chlorides, chloroformates, aldehydes, ketones, hydrazines, hydrazides, esters, sulphonyl chlorides, alcohols, phenols, azides, thiols, isocyanates, isothiocyanates, alkyl and aryl halides, epoxides, aziridines, enamines, acrylamides, enolethers, imidates, oximes, alkenes, acetylenes, amino groups, aniline groups, carboxylic groups and bifunctional groups having both an amine moiety and a carboxylic moiety.

37. (canceled)

38. The composition of claim 31, wherein at least some of the test molecules are not associated with an oligonucleotide.

39. The composition of claim 31, wherein each of substantially all of the target binding elements has a c Log P between −2 and 4.

40. The composition of claim 31, wherein each of substantially all of the target binding elements has 8 or fewer H-bond donors and optionally 4 or fewer H-bond acceptors.

41-42. (canceled)

43. The composition of claim 31, wherein each of substantially all of the target binding elements has 1 or more chiral centers.

44. A composition comprising a plurality of test molecules, wherein each of at least some of the test molecules comprises two or more target binding elements and is associated with a corresponding oligonucleotide having a nucleotide sequence that (i) identifies the two or more target binding elements, (ii) contains an amplification sequence, and (iii) is substantially incapable of hybridizing to the nucleotide sequences associated with other test molecules.

45-46. (canceled)

47. The composition of claim 44, wherein each of substantially all of the target binding elements has a K_{D} of 10 mM or less with a binding site.

48. The composition of claim 44, wherein for substantially all of the test molecules the product of the K_{D}'s with a binding site of the corresponding two or more target binding elements associated with the oligonucleotide corresponding to a test molecule is 10 mM or less.

49. The composition of claim 44, wherein each of substantially all of the target binding elements has a molecular weight between 90 and 500 daltons.

50. The composition of claim 44, wherein for substantially all of the test molecules the sum of the molecular weight of the corresponding two or more target binding elements associated with the oligonucleotide corresponding to a test molecule is between 120 and 400 daltons.

51. The composition of claim 44, wherein each of substantially all of the target binding elements is linked to a functional group selected from the group consisting of primary amines, secondary amines, primary anilines, carboxylic acids and a bifunctional groups having both an amine moiety and an acid moiety.

52. A complex of a target molecule bound to a test molecule comprising two or more target binding elements, wherein the test molecule is associated with a corresponding oligonucleotide having a nucleotide sequence that (i) identifies the test molecule and (ii) contains an amplification sequence, wherein each of substantially all of the target binding elements has at least one of the following characteristics: (i) a c Log P between −2 and 4, (ii) 4 or fewer H-bond donors, (iii) 8 or more H-bond acceptors, and (iv) a molecular weight between 90 and 500 daltons.

53. (canceled)

54. A composition comprising a plurality of complexes wherein each complex comprises a target molecule bound to a test molecule comprising two or more target binding elements, wherein each test molecule is associated with a corresponding oligonucleotide having a nucleotide sequence that (i) identifies the test molecule, (ii) contains an amplification sequence, and (iii) is substantially incapable of hybridizing to the nucleotide sequences of other test molecules.

55. The composition of claim 54, wherein each of substantially all of the target binding elements comprises a functional group through which the target binding element is attached to the oligonucleotide.

56-59. (canceled)

60. The composition of claim 54, wherein each of substantially all of the target binding elements has 1 or more chiral center.

61. The method of claim 1,
wherein each of substantially all of the target binding elements has at least one of the following characteristics: (i) a c Log P between −2 and 4, (ii) 4 or fewer H-bond donors, (iii) 8 or fewer H-bond acceptors, and (iv) a molecular weight between 90 and 500 daltons.

62-111. (canceled)

112. A method for identifying a compound having a desired binding affinity to a target molecule, the method comprising:
(a) providing a library comprising a plurality of test compounds, wherein each of the test compound comprises (1) a common binding moiety, (2) a scaffold moiety connected to the common binding moiety through a bridging moiety, and (3) an oligonucleotide having a nucleotide sequence informative of the structural or synthetic information of the associated test compound, wherein the common binding moiety has a dissociation constant of 10 mM or lower to a first binding domain of the target molecule;
(b) combining the target molecule and the plurality of test compound under conditions that permit binding of one or more of the plurality of test compounds to the target molecule if such test compounds with desired binding affinity are present;
(c) harvesting the test compounds bound to the target; and
(d) determining the oligonucleotide sequences of the test compounds harvested thereby identifying the test compounds having a desired binding affinity to the target molecule.

113-128. (canceled)
